(19) United States
(12) Patent Application Publication
(10) Pub. No.: US 2026/0235627 A1
(43) Pub. Date: Aug. 13, 2026

(54) BIOMOLECULES IN DISEASE

(71) Applicant: Imperial College Innovations Limited, South Kensington, London (GB)

(72) Inventors: Yu Ye, London (GB); Michael Morten, London (GB)

(21) Appl. No.: 19/116,888

(22) PCT Filed: Oct. 2, 2023
(86) PCT No.: PCT/GB2023/052540

§ 371 Date: Mar. 28, 2025

(30) Foreign Application Priority Data
Sep. 30, 2022 (GB) 2214417.4

(51) Int. Cl. G01N 33/68; G06V 10/764; G06V 10/82; G06V 20/69; G16B 40/20; G16B 40/30

(52) U.S. Cl. CPC G01N 33/6896; G06V 10/764; G06V 10/82; G06V 20/698; G16B 40/20; G16B 40/30; G01N 2800/2814; G01N 2800/2821; G01N 2800/2835; G01N 2800/50; G01N 2800/52; G06V 2201/03

(57) ABSTRACT

The invention relates to methods of screening for the presence of proteopathies, to methods of diagnosing proteopathies, to methods of differentially diagnosing proteopathies, to methods of assessing the severity, stage and/or prognosis of proteopathies, and to methods for monitoring the progression of proteopathies. The invention also relates to methods for determining the efficacy of therapeutic interventions for proteopathies.

### FIELD OF THE INVENTION

The invention relates to methods of screening for the presence of proteopathies, to methods of diagnosing proteopathies, to methods of differentially diagnosing proteopathies, to methods of assessing the severity, stage and/or prognosis of proteopathies, and to methods for monitoring the progression of proteopathies. The invention also relates to methods for determining the efficacy of therapeutic interventions for proteopathies.

### BACKGROUND

Proteopathies (also known as proteinopathies) are diseases and conditions that are associated with the abnormal production, folding, aggregation, or degradation of proteins. Proteopathies may also be referred to as protein misfolding diseases. Protein aggregation is the accumulation or clumping together of misfolded proteins, leading to the formation of abnormal, toxic cell aggregates that lead to cellular damage or death. The more these aggregates accumulate, the more severe the illness becomes. Various proteopathies are associated with protein aggregates. During protein aggregation, small numbers of misfolded proteins initially associate to form oligomers. Oligomers coalesce and are elongated by aggregation of more misfolded proteins. Small aggregates undergo conformational changes to produce proto-fibrils, eventually forming the larger insoluble fibrils found in Lewy bodies, neurofibrillary tangles, and plaques commonly associated with various proteopathies. Aggregation occurs in three stages: the lag phase, the growth phase, and plateau phase (FIG. 31).

Diagnostic and therapeutic research has typically focused on large insoluble aggregates (e.g. plaques and Lewy bodies) which are hallmarks of various diseases, including major neurodegenerative disorders, such as Alzheimer's disease (AD) and Parkinson's disease (PD). However, much of this research relies on examination of brain tissue which can only be performed post-mortem. Moreover, protein aggregation is a natural phenomenon and protein aggregates are common to both healthy and diseased individuals. In addition, the same type of protein aggregates are typically common to a range of proteopathies and so associating aggregates with a particular disease is challenging.

As with many diseases and disorders, early therapeutic interventions for proteopathies may have greater efficacy than interventions during later disease stages. However, current diagnostic tests are not sufficiently accessible, sensitive or reliable to detect proteopathies before the onset of symptoms thereby limiting the ability to identify candidates for early therapeutic intervention.

Diagnosis of neurodegenerative diseases is typically a lengthy process which relies on subjective analysis of symptoms (e.g. memory tests) and medical imaging techniques that suffer from low accessibility and low specificity. Positron emission tomography (PET) and single-photon emission computerized tomography (SPECT) scans have historically been the gold-standard for clinical diagnosis, but these methods are too expensive for use in routine screening and involve exposure to radiation. In many instances conclusive diagnosis occurs only during post-mortem analysis.

Dementia is a proteopathy syndrome which affects over 55 million people worldwide, and this figure is expected to triple by 2050. There are various types of dementia, each having varying prognoses and symptoms. There are currently no reliable, clinically scalable methods for differentiating between different diseases within dementia. As disease modifying therapies are beginning to be introduced for some types of dementia, reliable diagnosis of disease, disease progression and disease stage, becomes increasingly important.

Dementia is one of the leading causes of impairment and dependency among older people worldwide. Initial signs that may emerge well before dementia is diagnosed include challenges with memory, trouble focusing, and difficulties in executing everyday tasks, such as accurately handling money during shopping. Additionally, individuals might find it challenging to keep up with conversations, search for appropriate words, experience disorientation concerning time and location, and undergo mood fluctuations. These symptoms, while subtle, can progressively intensify. Often, this stage is labeled as “mild cognitive impairment” (MCI) since the symptoms aren't pronounced enough to classify as dementia. The term dementia encapsulates a wide spectrum of neurodegenerative diseases, characterized by the progressive deterioration of cognitive functions such as memory, thinking, orientation comprehension, calculation, learning capacity, and judgment.

Protein aggregation is a critical feature of many neurodegenerative diseases, collectively known as proteopathies. Notably, among these diseases are tauopathies and synucleinopathies. Tauopathies are a group of neurodegenerative conditions that are distinguished by the accumulation of aberrant tau proteins in the brain. This category of conditions includes AD and Frontotemporal dementia (FTD). Synucleinopathies are a varied collection of neurodegenerative illnesses that share a common pathological defect consisting of clumps of insoluble alpha-synuclein protein in selectively susceptible populations of neurons and glia. Synucleinopathies include Dementia with Lewy bodies (DLB) and PD.

In order to detect proteopathies, protein aggregates have been used as biomarkers, or measurable indicators of the severity or presence of a disease. The most sensitive methods are able to detect individual molecules. Single-molecule array (SIMOA) technology can detect trace amounts of critical proteins such as alphasynuclein and tau. By identifying biomarkers associated with neurodegenerative diseases, SIMOA enhances early diagnosis and monitoring. However, there are currently no specific biomarkers that can effectively distinguish between different types of dementia.

There exists an urgent and unmet need for diagnostic methods that can detect the presence of a proteopathy or an increased risk thereof in a subject. In particular, there is an urgent and unmet need for diagnostic methods that can be used to diagnose and screen for proteopathies, or an increased risk thereof, particularly early in disease progression, such as before the onset of symptoms.

### SUMMARY OF THE INVENTION

The inventors have overcome the above problems by identifying a sub-population of protein aggregates that can be used to detect and/or diagnose the presence of a proteopathy. Advantageously, these toxic protein aggregates also enable the differential diagnosis of proteopathies, even when said proteopathies involve aggregation of the same protein.

A key advantage of the methods of the invention is the ability to identify proteopathy-associated toxic protein aggregates even at very low concentrations. A further advantage of methods of the invention is that they accurately and reliably detect and/or diagnose proteopathy in living patients. A particular advantage is that the methods of the invention can be used to screen for and/or diagnose the presence of proteopathies, or a risk thereof, before the onset of symptoms. Importantly, the methods of the invention can be performed using bodily fluid samples obtained from a patient thereby ensuring that the methods of the invention are significantly less invasive than existing diagnostic methods, e.g. PET and SPECT scans. The methods of the invention are also simpler, more sensitive and more accurate than diagnostic methods currently in use.

In one embodiment, the invention provides a method of screening for the presence of a proteopathy or an increased risk thereof in a patient, the method comprising: (a) comparing the morphology, toxicity and/or abundance of protein aggregates in a patient sample to a control; and (b) determining if the patient has a proteopathy or an increased risk thereof based on the comparison performed in step (a); wherein the protein aggregates are up to 1 μm in size. In some embodiments, comparing the morphology of protein aggregates comprises comparing one or more structural parameters of the protein aggregates.

In one embodiment, the invention provides a method of diagnosing a proteopathy in a patient, the method comprising comparing the morphology, toxicity and/or abundance of protein aggregates in a patient sample to a control associated with a distinct proteopathy, wherein: (i) a difference in the morphology, toxicity and/or abundance of protein aggregates between the patient sample and the control indicates the absence of the proteopathy associated with that control; or (ii) no difference in the morphology, toxicity and/or abundance of protein aggregates between the patient sample and the control indicates the presence of the proteopathy associated with that control; and wherein the protein aggregates are up to 1 μm in size. In some embodiments, comparing the morphology of protein aggregates comprises comparing one or more structural parameters of the protein aggregates.

In one embodiment, the invention provides a method of assessing the severity, stage and/or prognosis of a proteopathy in a patient, the method comprising: (a) comparing the morphology, toxicity and/or abundance of protein aggregates in a patient sample to one or more control(s), wherein the one or more control(s) is associated with a known severity, stage and/or prognosis of proteopathy; and (b) determining the severity, stage and/or prognosis of the proteopathy based on the comparison performed in step (a); wherein the protein aggregates are up to 1 μm in size. In some embodiments, comparing the morphology of protein aggregates comprises comparing one or more structural parameters of the protein aggregates.

In one embodiment, the invention provides a method for monitoring the progression of a proteopathy in a patient, the method comprising: (a) comparing: (i) the morphology, toxicity and/or abundance of the protein aggregates in a patient sample obtained at a first time point with (ii) the morphology, toxicity and/or abundance of the protein aggregates in a patient sample obtained at a second subsequent time point; and (b) determining whether the proteopathy has progressed between the first and second time point based on the comparison performed in step (a); wherein the protein aggregates are up to 1 μm in size. In some embodiments, comparing the morphology of protein aggregates comprises comparing one or more structural parameters of the protein aggregates.

In one embodiment, the invention provides a method for determining the efficacy of a therapeutic intervention in a patient having a proteopathy, the method comprising: (a) comparing: (i) the morphology, toxicity, and/or abundance of protein aggregates in a patient sample obtained prior to a therapeutic intervention with (ii) the morphology, toxicity, and/or abundance of protein aggregates in a patient sample obtained after the therapeutic intervention; and (b) determining the efficacy of the therapeutic intervention based on the comparison performed in step (a); wherein the protein aggregates are up to 1 μm in size. In some embodiments, comparing the morphology of protein aggregates comprises comparing one or more structural parameters of the protein aggregates.

In some embodiments, the proteopathy is a neurodegenerative disease. In some embodiments, the neurodegenerative disease is selected from Dementia with Lewy bodies, Parkinson's disease, Alzheimer's disease, frontotemporal dementia, Frontotemporal lobar degeneration, vascular dementia, Creutzfeldt-Jakob disease, amyloidosis, a trinucleotide repeat disorder (such as Huntington's disease), Amyotrophic lateral sclerosis (ALS), and a prion disease (such as bovine spongiform encephalopathy).

In some embodiments, the protein aggregate is an amyloid-beta aggregate, an alpha-synuclein aggregate, and/or a tau aggregate. In some embodiments, the protein aggregate is a superoxide dismutase 1 (SOD1) aggregate, a TAR DNA-binding protein 43 (TDP-43) aggregate, a huntingtin (HTT) aggregate, and/or a prion/PrP aggregate. In some embodiments, the protein aggregate is an amyloid-beta aggregate, an alpha-synuclein aggregate, a tau aggregate, a SOD1 aggregate, a TDP-43 aggregate, a HTT aggregate, and/or a prion/PrP aggregate.

In some embodiments, method comprises comparison with two or more control(s), wherein the two or more control(s) comprise: (i) a control associated with Dementia with Lewy bodies; (ii) a control associated with Parkinson's disease; (iii) a control associated with Alzheimer's disease; and/or (iv) a control associated with frontotemporal dementia.

In some embodiments, the proteopathy is selected from cancer, diabetes, cardiovascular disease, cystic fibrosis, sickle cell disease, and protein aggregate myopathies. In some embodiments, the protein aggregates is selected from a tumour suppressor protein p53 aggregate, an islet amyloid polypeptide aggregate, a cystic fibrosis transmembrane conductance regulator (CFTR) aggregate, a haemoglobin aggregate and a myosin aggregate.

In some embodiments, the proteopathy is selected from amyloidosis. In some embodiments, the protein aggregates is selected from an immunoglobin aggregate, an Amyloid A protein aggregate, an aggregate containing a member of apolipoproteins, a gelsolin aggregate, a lysozyme aggregate, a fibrinogen aggregate, a microglobulin aggregate, a transthyretin aggregate, a keratin aggregate, a lactoferrin aggregate a corneodesmosin aggregate and an enfuvirtide aggregate.

In some embodiments, the method comprises detecting and comparing protein aggregates up to 600 nm in size, optionally up to 550 nm, 540 nm, 530 nm, 520 nm, 510 nm, 500 nm, or 450 nm in size.

In some embodiments, the method comprises detecting and comparing protein aggregates that are up to 450±60 nm in size.

In some embodiments, the sample is selected from cerebrospinal fluid, blood, serum, plasma, faeces, urine, or a biopsy sample.

In some embodiments, protein aggregates are detected using a high resolution microscopy method, optionally a super-resolution microscopy method. In some embodiments, the high resolution microscopy method is selected from Single-molecule localization microscopy (SMLM), cryogenic electron microscopy, atomic force microscopy, total-internal reflection fluorescence (TIRF) microscopy, Stochastic Optical Reconstruction Microscopy (STORM), Photoactivated Localization Microscopy (PALM), Stimulated Emission Depletion (STED) microscopy, transmission electron microscopy, structured illumination microscopy, light-sheet microscopy, scanning probe microscopy, confocal microscopy, two-photon microscopy, and fluorescence lifetime imaging.

In some embodiments, protein aggregates are detected in two-dimensions and three dimensions and in two-dimensions or three dimensions over time.

In some embodiments, the method comprises quantifying morphological features of the protein aggregates; and comparing said morphological features to a control. In some embodiments, the morphological features comprises one or more of the area, eccentricity, solidity, skeleton size, and number of branches of the protein aggregates.

In some embodiments, the morphological features comprise one or more of the area, solidity, skeleton size, and number of branches of the protein aggregates. In some embodiments, the morphological features comprise the area, solidity, skeleton size, and number of branches of the protein aggregates.

In some embodiments, the method comprises quantifying the toxicity of the protein aggregates by: (a) performing a cell stress, viability and/or cytotoxicity assay (such as a lactate dehydrogenase (LDH) assay, a (2′,7′-dichlorofluorescin diacetate) DCFDA assay, and/or an Amplex Red assay); and/or (b) detecting proteasome foci formation.

In some embodiments, the method comprises comparing the relative abundance of the protein aggregates in the patient sample to a control, optionally wherein the abundance of the protein aggregates is determined relative to the total abundance of protein aggregates in the patient sample.

In some embodiments, the method comprises using statistical analysis to compare the morphology, toxicity and/or abundance of protein aggregates, optionally wherein the statistical analysis comprises a neural network, a random forest model, and/or logistic regression.

In one embodiment, the invention provides a computer-implemented neural network for classifying protein aggregates, for screening for or diagnosing the presence of a proteopathy or an increased risk thereof in a patient, the neural network comprising: an input layer for receiving an input corresponding to an image of a protein aggregate; at least one hidden layer connected to the input layer; and an output layer connected to the at least one hidden layer; wherein the output layer is configured for outputting an indication of whether the protein aggregate is associated with a proteopathy; and wherein the image of the protein aggregate is an image of a protein aggregate that is up to 1 μm in size.

In one embodiment, the invention provides a computer-implemented neural network for assessing the severity, stage and/or prognosis of a proteopathy in a patient, the neural network comprising: an input layer for receiving an input corresponding to an image of a protein aggregate; at least one hidden layer connected to the input layer; and an output layer connected to the at least one hidden layer; wherein the output layer is configured for outputting an indication of the severity, stage and/or prognosis associated with the protein aggregate; and wherein the image of the protein aggregate is an image of a protein aggregate that is up to 1 μm in size.

In some embodiments, the computer-implemented neural network is a convolutional neural network.

In some embodiments, the image of the protein aggregate is an image of a single protein aggregate.

In some embodiments, the output layer is configured for outputting an indication of a type of proteopathy associated with the protein aggregate. In some embodiments, the output layer is configured for outputting an indication of the severity, stage and/or prognosis associated with the protein aggregate.

In some embodiments, the neural network is trained using training data based on: images of protein aggregates from at least one healthy sample; and images of protein aggregates from at least one sample associated with a proteopathy.

In some embodiments, wherein the method comprises assessing the severity, stage and/or prognosis of a proteopathy in a patient, the method further comprises training the neural network using training data based on: images of protein aggregates from patients at different stages or levels of disease severity; or from different age groups.

In some embodiments, the image of the protein aggregate is an image of a protein aggregate that is up to 600 nm in size, optionally up to 550 nm, 540 nm, 530 nm, 520 nm, 510 nm, 500 nm, or 450 nm in size.

In one embodiment, the invention provides a computer-implemented method for classifying protein aggregates, for screening for or diagnosing the presence of a proteopathy or an increased risk thereof in a patient, the method comprising: receiving, in an input layer of a neural network, an input corresponding to an image of a protein aggregate, wherein the neural network has at least one hidden layer connected to the input layer and an output layer connected to the at least one hidden layer; determining, using the neural network, whether the protein aggregate is associated with a proteopathy; and outputting an indication of whether the protein aggregate is associated with a proteopathy; wherein the image of the protein aggregate is an image of a protein aggregate that is up to 1 μm in size.

In one embodiment, the invention provides a computer-implemented method for assessing the severity, stage and/or prognosis of a proteopathy in a patient, the method comprising: receiving, in an input layer of a neural network, an input corresponding to an image of a protein aggregate, wherein the neural network has at least one hidden layer connected to the input layer and an output layer connected to the at least one hidden layer; determining, using the neural network, the severity, stage and/or prognosis of proteopathy associated with the protein aggregate; and outputting an indication of the severity, stage and/or prognosis associated with the protein aggregate; wherein the image of the protein aggregate is an image of a protein aggregate that is up to 1 μm in size.

The invention also provides a computer-implemented method for classifying protein aggregates, for screening for or diagnosing the presence of a proteopathy or an increased risk thereof in a patient, the method comprising: receiving an input corresponding to an image of a protein aggregate; determining, using a classification algorithm and the input, whether the protein aggregate is associated with a proteopathy; and outputting an indication of whether the protein aggregate is associated with a proteopathy; wherein the image of the protein aggregate is an image of a protein aggregate that is up to 1 μm in size.

The invention also provides a computer-implemented method for assessing the severity, stage and/or prognosis of a proteopathy in a patient, the method comprising: receiving an input corresponding to an image of a protein aggregate; determining, using a classification algorithm and the input, the severity, stage and/or prognosis of proteopathy associated with the protein aggregate; and outputting an indication of the severity, stage and/or prognosis associated with the protein aggregate; wherein the image of the protein aggregate is an image of a protein aggregate that is up to 1 μm in size.

In some embodiments, the neural network is a convolutional neural network.

In some embodiments, the method further comprises training the neural network using training data based on: images of protein aggregates from at least one healthy sample; and images of protein aggregates from at least one sample associated with a proteopathy.

In some embodiments, wherein the method comprises assessing the severity, stage and/or prognosis of a proteopathy in a patient, the method further comprises training the neural network using training data based on: images of protein aggregates from patients at different stages or levels of disease severity; or from different age groups.

In some embodiments, the method comprises: receiving, in the input layer of the neural network, inputs corresponding to a plurality of images, each image being an image of a respective protein aggregate, determining, using the neural network, whether each of the protein aggregates is associated with a proteopathy; and outputting, based on the number of the protein aggregates determined to be associated with a proteopathy, an indication of a level of cytotoxicity.

In some embodiments, the method comprises performing principal component analysis using the input to generate a set of variables, and determining whether the protein aggregate is associated with a proteopathy using the classification algorithm and the set of variables.

In some embodiments, the method comprises: receiving the input in an input layer of an autoencoder, wherein the autoencoder is configured to generate an autoencoder output using the image of the protein aggregate; and determining whether the protein aggregate is associated with a proteopathy by using the classification algorithm and the autoencoder output.

In some embodiments, the dimensionality of the autoencoder output is lower than the dimensionality of the input.

In some embodiments, the method comprises performing principal component analysis using the autoencoder output to generate a set of variables, and determining whether the protein aggregate is associated with a proteopathy using the classification algorithm and the set of variables.

In some embodiments, determining whether the protein aggregate is associated with a proteopathy using the classification algorithm comprises performing a nearest neighbour search using the set of variables.

In one embodiment, the invention provides a computer-implemented method for classifying protein aggregates, for screening for or diagnosing the presence of a proteopathy or an increased risk thereof in a patient, method comprising: obtaining, based on an image of a protein aggregate, two or more morphological features of the protein aggregate; determining, using a computer-implemented classification algorithm, based on the two or more morphological features of the protein aggregate, whether the protein aggregate is associated with a proteopathy; and outputting an indication of whether the protein aggregate is associated with a proteopathy; wherein the image of the protein aggregate is an image of a protein aggregate that is up to 1 μm in size.

In one embodiment, the invention provides a computer-implemented method for assessing the severity, stage and/or prognosis of a proteopathy in a patient, method comprising: obtaining, based on an image of a protein aggregate, two or more morphological features of the protein aggregate; determining, using a computer-implemented classification algorithm, based on the two or more morphological features of the protein aggregate, the severity, stage and/or prognosis associated with the protein aggregate; and outputting an indication of the severity, stage and/or prognosis associated with the protein aggregate; wherein the image of the protein aggregate is an image of a protein aggregate that is up to 1 μm in size.

In some embodiments, the morphological features comprise two or more of: an area of the protein aggregate; an eccentricity of the protein aggregate; a proportion of pixels inside a convex hull fitted to the protein aggregate; a number of pixels obtained in a process in which border pixels from an image of the protein aggregate are removed until no more can be removed without breaking connectivity of pixels corresponding to the protein aggregate; or a number of branches in the image of the protein aggregate after border pixels have been removed until no more can be removed without breaking connectivity of the pixels corresponding to the protein aggregate.

In some embodiments, the number of pixels obtained in a process in which border pixels are removed until no more can be removed without breaking connectivity of the pixels corresponding to the protein aggregate is a skeleton size obtained by applying a skeletonization process to an image of the protein aggregate to obtain a skeletonized image.

In some embodiments, the number of branches is the number of branching points of the protein aggregate in the skeletonized image.

In some embodiments, the classification algorithm is a random forest algorithm.

In some embodiments, the method further comprises training the classification algorithm using training data based on: images of protein aggregates from at least one healthy sample; and images of protein aggregates from at least one sample associated with a proteopathy.

In some embodiments, wherein the method comprises assessing the severity, stage and/or prognosis of a proteopathy in a patient, the method further comprises training the neural network using training data based on: images of protein aggregates from patients at different stages or levels of disease severity; or from different age groups.

In some embodiments, the method comprises: obtaining the two or more morphological features for a plurality of protein aggregates, determining, using the computer-implemented classification algorithm, based on the two or more morphological features of each protein aggregate, whether each protein aggregate is associated with a proteopathy; and outputting, based on the number of the protein aggregates determined to be associated with a proteopathy, an indication of a level of cytotoxicity.

In one embodiment, the invention provides a system for classifying protein aggregates, for screening for or diagnosing the presence of a proteopathy or an increased risk thereof in a patient, the system comprising: an image capturing device for capturing an image of a protein aggregate; and a computing device for receiving the image from the image capturing device, wherein the computing device is configured to perform the method set out in any one of the embodiments above; wherein the image of the protein aggregate is an image of a protein aggregate that is up to 1 μm in size.

In one embodiment, the invention provides a system for assessing the severity, stage and/or prognosis of a proteopathy in a patient, the system comprising: an image capturing device for capturing an image of a protein aggregate; and a computing device for receiving the image from the image capturing device, wherein the computing device is configured to perform the method set out in any one of the embodiments above; wherein the image of the protein aggregate is an image of a protein aggregate that is up to 1 μm in size.

In some embodiments, the image capturing device is a high-resolution imaging device, optionally a super-resolution imaging device.

In one embodiment, the invention provides a computer program comprising instructions which, when the program is executed by a computer, cause the computer to carry out the method of any one of the embodiments above.

### DESCRIPTION OF THE FIGURES

FIG. 1. Classification apparatus 10 for classifying protein aggregates.

FIG. 2. Representation of a method of classification that may be performed by the classification apparatus 10.

FIG. 3. (A) Diffraction limited field of view of aggregates on a coverslip, (B) SMLM image of single aggregate after pre-processing. x- and y-axis are labelled with the number of pixels in each image; (C) exemplary aggregates from DLB, PD, and healthy controls.

FIG. 4. Convolutional neural network (CNN) architecture.

FIG. 5. (A) CNN training and validation accuracy and loss curves; (B) ROC curve for CNN; c, violin plot showing the spread of CNN prediction confidence by class.

FIG. 6. Exemplary aggregates from each class that were correctly classified with high confidence.

FIG. 7. Box-plots showing difference in shape feature between diseases, with results from independent two sample t-test with bonferroni correction. Shape features are: (A) area; (B) eccentricity; (C) solidity; (D) number of branches; and (E) skeleton size. Measures are averaged by patient to prevent independence violation.

FIG. 8. Spider web plot showing average levels of each shape feature in each disease class.

FIG. 9. (A) Accuracy by patient for random forest and CNN; (B) ROC curve for random forest; (C) Random forest performance metrics.

FIG. 10. Permutation-based importance of features in random forest.

FIG. 11. Association between the proportion of PD predicted aggregates and normalised cytotoxicity. Cytotoxicity is normalised by the proportion of aggregates under 450 nm.

FIG. 12. Detecting aggregate species with ThT, ProteoStat and AT630. (A) Schematic representation of small aggregate and fibril detection with fluorophores that reversibly bind to aggregates. Unbound fluorophores are low- or non-fluorescent. Binding to aggregates locks the fluorophores in a high-fluorescence conformation (see bound fluorophores), from which aggregate morphology can be detected. (B) Representative TIRF images of aggregates assembled from recombinant αS stained with ThT and ProteoStat (top) or ThT and AT630 (bottom). Laser power was set at 10 mW unless otherwise stated. Scale bars represent 5 μm. (C) TIRF images of typical aggregates ranging from 0.2 to 5 μm in size detected with ThT (top), ProteoStat (middle) and AT630 (bottom). Scale bars represent 1 μm. (D) Semi-log plot of frequency count of aggregates detected up to 60 μm with each fluorophore. Kolmogorov-Smirnov test indicates that AT630 and Proteostat detect a significantly greater number of aggregates (N_{AT630}=4417; N_{ProteoStat}=4473; N_{ThT}=2353) and are more faithful at detecting larger aggregates as evidenced by their greater medians and interquartile ranges, IQR (median(IQR)_{AT630}=2428(655-7260) nm; median (IQR)_{ProteoStat}=1763 (452-5712) nm; median(IQR)_{ThT}=1499 (490-4432) nm; p<0.0005). See FIG. 13 for biophysical properties of the three fluorophores.

FIG. 13. Photophysical properties of ProteoStat and AT630. (A) UV-visible absorption and emission spectra of ThT, ProteoStat and AT630 were measured on a spectrophotometer. Average spectra from three independent measurements are shown. Emission peak, λ_{Em}, for ThT (λ_{Em}=400 nm) was found at 484 nm; ProteoStat emission λ_{Em}=610 nm (λ_{Ex}=500 nm); and AT630 emission λ_{Em}=642 nm (λ_{Ex}=480 nm). (B) Quantum yields and extinction coefficients of ThT, ProteoStat and AT630 were determined as described in Materials and Methods and from which brightness were calculated. Alexa647 quantities are taken from the manufacturer (ThermoFisher Scientific), and ThT quantum yield is comparable to that previously observed in water.

FIG. 14. AT630 and ProteoStat preferentially bind to aggregate structures. (A) αS monomers labeled with Alexa647 could not be detected when stained with AT630 or ProteoStat. (B) Representative images showing that unstained αS aggregates are not detected without AT630 or ProteoStat staining. Scale bars represent 5 μm.

FIG. 15. ProteoStat and AT630 recognize Alexa647-labeled aggregates. (A) Schematic representation of aggregate labeling with Alexa647. Recombinant unlabeled and Alexa647-labeled aS monomers were mixed in an optimized ratio of 9:1 prior to aggregation (see Materials and Methods). This method ensures that Alexa647-labeled &S are incorporated into all aggregate species (not drawn to scale). (B) Reconstruction of an Alexa647-labeled fibril in 3D, assembled according to A. Scale bar=500 nm. (C) Alexa647-labeled aggregates were stained with either ThT (top), ProteoStat (middle) or AT630 (bottom) and fluorescence emissions from both channels detected simultaneously. Laser power was set at 10 mW for ProteoStat and AT630 and 60 mW for ThT. Scale bars=2 μm. (D) The relationship between fluorescence intensity and size of each aggregate is plotted, color-coded by the fluorophore used for detection (N_{AT630}=136; N_{ProteoStat}=206; N_{ThT}=474; N_{Alexa647}=213).

FIG. 16. Characterization of 2D and 3D SMLM techniques. (A) The resolution of the 2D SMLM was calculated by imaging αS monomers labeled with Alexa647 deposited on a plasma cleaned coverslip. The relative positions in the x- and y-directions were calculated from multiple bursts of fluorescence from individual fluorophores, and were plotted in frequency histograms. The FWHM from each histogram represent the possible resolution of the 2D SMLM (N=17874 fluorescent bursts). (B) Similarly, from astigmatic SMLM, the positions in three dimensions can be plotted in a 3D scatter plot, and are quantified in the x-, y- and z-directions in (C) (N=3741 fluorescent bursts).

FIG. 17. Aggregates assembled from αS covalently linked with Alexa647 or Alexa488. (Left) Unlabeled and Alexa488-or Alexa647-labeled αS monomers were mixed in a 9:1 ratio and assembled into aggregates. (Right) Representative (A) Alexa647—and (B) Alexa488-linked aggregates detected by diffraction-limited or super-resolved TIRF microscopy. Scale bars=2 μm.

FIG. 18. Super-resolution of aggregates by single-molecule localization microscopy (SMLM) with ProteoStat and AT630. (A) The principle of point spread function (PSF) analysis. Emission from Alexa647 is detected as a diffraction-limited spot in 2D (bottom). The emission may be presented in 3D using fluorescence intensities registered at each pixel (middle). The PSF is mathematically modeled to pinpoint a more precise location of the fluorophore (top). (B) The average PSFs detected from SMLM imaging of aggregates using Alexa488, ProteoStat, AT630 and ThT. (C) Table of values for measured localization precision (Δloc) calculated from the PSF of each fluorophore, using Δloc=FWHM/\sqrt{photons}. (D) (left) Comparison of recombinant AT630 and ProteoStat-stained αS aggregates imaged by SMLM reconstruction and conventional diffraction-limited TIRF methods. Scale bars represent 2 μm. (right) Representative images of smaller aggregates detected using SMLM. Scale bars represent 100 nm. (E) An example of improvement in determining distribution of aggregates size as measured from diffraction-limited or super-resolution images (in the rectangle) using AT630 (N=1273 for the diffraction limited and N=576 for the SMLM samples).

FIG. 19. ProteoStat-stained Aβ and tau aggregates. (A) Aggregates assembled from synthetic Aβ42 or (B) recombinant tau were stained with AT630 and detected by TIRF microscope, shown as diffraction-limited or super-resolved image (SMLM). Close-ups on typical features of aggregates detected in A are shown on the right. Aggregates assembled from recombinant full-length tau (0N4R) carry the P301S mutation. (D-E) Similarly, Aβ42 and tau aggregates are imaged with ProteoStat. Scale bars in A and C represent 1 μm and in B and D 100 nm.

FIG. 20. The cell volume observed using eGFP emission from a typical HEK293A cell expressing proteasome subunit PSMD14-eGFP and compared to the cell volume highlighted using CellMask™ plasma membrane stain. Cells are shown in (A) orthogonal views and (B) 3D representation of the cell. Scale bar represents 5 μm.

FIG. 21. Imaging cell-penetrating aggregate species with size less than 450±60 nm. (A) αS aggregates at 1 μM were added to HEK293A cells expressing proteasome subunit PSMD14-eGFP from the genomic loci. Aggregates were stained with AT630. A typical cell is shown with profiles along the z-axis and aggregates detected within the cell interior or sitting on the external side of plasma membrane as indicated. Scale bars represent 5 μm. (B) Quantification of aggregates detected in live cells (N₄ₕ=14 cells; N₁₂ₕ=13 cells) within the intracellular domain after 4 hrs or 12 hrs after incubation. The dashed line indicates the intersection of the 4 hrs and 12 hrs curves at 450±60 nm. Aggregate size distribution of the starting sample at time 0 hr is shown (N₄ₕ=529; N₁₂ₕ=585; N₀ₕ=2734). (C) Live super-resolution imaging by SMLM of AT630-stained aggregates in the intracellular domain or blocked by the plasma membrane are shown. Proteasome foci are represented by bright spots. Typical foci-aggregate colocalizations are shown in the zoom-in images. Scale bars represent 2 μm and 200 nm for the whole cell and insets, respectively. (D) Quantification of aggregates detected by SMLM, showing the relative frequencies of aggregates with sizes between 0-100 nm. Aggregates from 19 cells were imaged, with 893 total number of aggregates. Sonicated aggregates were imaged from 5 samples, with a total of 849 aggregates. A two-sample t-test was used to calculate statistical significance between the groups of sizes, with n.s. (no significance) p>0.05, *p<0.05 and ** p<0.005.

FIG. 22. AT630 enables to visualization of aggregates in cells. (left) HEK293A cells expressing proteasome subunit PSMD14-eGFP were treated with 1 μM unlabeled αS aggregates and were imaged without staining with AT630. (right) HEK293A cells expressing proteasome subunit PSMD14-eGFP were stained with AT630 in the absence of aggregates. Scale bars represent 5 μm, and no significant signal is detected from either sample.

FIG. 23. Quantitative super-resolution imaging correlates aggregate₄₅₀ₙₘ with toxicity. (A) The aggregation process of recombinant αS over time as measured by ThT. Arrows below cartoons represent the aggregate species (not drawn to scale) detected by TIRF imaging of aliquots taken at indicated timepoints (see FIG. 24). (B) Representative small aggregates detected using AT630 at 12 hrs timepoint after aggregation. Scale bars represent 100 nm. (C) Quantification of aggregate species by size as detected by super-resolution for timepoints at 12 and 36 hrs, and sonicated aggregates (N₁₂ₕ =313; N₃₆ₕ=595; Nₛₒₙ=287). The horizontal dashed line shows the 450 nm size threshold calculated in FIG. 21B. (D) A typical AT630-stained αS aggregate reconstructed in 3D from super-resolution imaging, x-y resolution 24 nm and z resolution 48 nm. Scale bars represent 500 nm in x, y and z directions. (E) Quantification of αS aggregate cytotoxicity as determined by LDH assay for aliquots taken and the timepoints indicated in A. Cells were treated with a final concentration of 1 μM αS. Error bars represent standard deviation of triplicate measurements (n=3). (F) Cytotoxicity-to-aggregate₄₅₀ₙₘ normalized to aggregate concentration shows a linear relationship.

FIG. 24. Typical TIRF images of aliquots taken from αS aggregation reaction. Aggregates were stained with ProteoStat and imaged at (A) 0, (B) 12 and (C) 36 hrs of aggregation. (D) An image of sonicated sample from C. All images were set to the same intensity range. Scale bars=5 μm.

FIG. 25. Brain soak aggregate extraction from post-mortem PD and DLB donor brain tissues. (A) Cartoon representation of the experimental workflow for extraction of aggregates from donor brain tissues. (B) Aggregates extracted from three PD and three DLB samples were separated on a 4-12% SDS-PAGE and stained with Coomassie (see Table 3). Ladder (L) for molecular weights is indicated. (C) Samples from B were transferred to a PVDF membrane and stained with an anti-aS antibody (MJRF1). (D) The brain soak samples were separated on a 4-16% NativePAGE and stained with Coomassie.

FIG. 26. Aggregates derived from post-mortem PD and DLB donors are quantitively detected by SMLM using AT630 staining. (A) Features of typical small aggregates detected in PD samples are shown. Scale bar=100 nm. (B) Quantification of super-resolved aggregates by size in each donor sample is shown, with mean±SEM sizes of 140±10 nm, 211±7 nm and 106±2 nm for PD1, PD2 and PD3 respectively (N_{PD1}=599; N_{PD2}=10084; N_{PD3}=8663). The horizontal dashed line shows the 450 nm size threshold calculated in FIG. 21B. (C) LDH assay performed similar to HEK293A experiment in FIG. 23F, where cytotoxicity values are plotted against the calculated concentration of aggregate₄₅₀ₙₘ. Increasing concentrations of PD samples were used to determine the linear range (see also FIG. 27) which are plotted here, and are fitted to a straight line (dashed line). The plotted values are in agreement with the toxicity-to-aggregate₄₅₀ₙₘ relationship determined in FIG. 23F (solid line). The fraction of aggregate₄₅₀ₙₘ found in the samples for PD1, PD2 and PD3 were 11%, 12% and 20%, respectively. (D) Features of typical aggregates detected in DLB samples are shown. Scale bar=100 nm. (E) Quantification of super-resolved aggregates by size in each donor sample is shown, with mean sizes±SEM 200±20 nm, 66±2 nm and 109±2 nm respectively for DLB1, DLB2 and DLB3 samples (N_{DLB1}=1446; N_{DLB2}=8009; N_{DLB3}=11866). The horizontal dashed line shows the 450 nm size threshold calculated in FIG. 21B. (F) Cytotoxicity levels of three DLB samples plotted against the concentration of aggregate₄₅₀ₙₘ. The solid line represents the linear relationship in FIG. 23F determined from recombinant αS samples, and the dashed line represents a linear relationship of the DLB samples. The fraction of aggregate₄₅₀ₙₘ in DLB1, DLB2 and DLB3 are 43%, 84% and 69%, respectively. (G) Live HEK293A cells expressing PSMD14-eGFP were also incubated with 1 μM PD1 patient derived aggregates and stained with AT630. (H) Quantification of PD1 aggregates within intracellular domain (N_{PD1,4h}=4740 aggregates inside 115 cells). The black dashed line is taken from analogous experiment shown in B where cells were incubated with αS aggregates for 4 hrs.

FIG. 27. The relationship between cytotoxicity and aggregate₄₅₀ₙₘ concentrations of PD1, PD2 and PD3 (see Table 3), show a linear relationship between 0-1 UM range (dashed line), and compared to the linear fit (solid line) described in FIG. 23F for the recombinant αS samples.

FIG. 28. Cytotoxicity from LDH assay is not altered by prolonged incubation and independent of fibril concentration. (A) The cytotoxicity levels of the PD and DLB samples at 4 hrs and 24 hrs (black and grey bars, respectively) show no significant (n.s.) difference in cytotoxicity between samples incubated for the two different times. Mean values and error bars represent standard deviation of three independent measurements. (B) (left) The relationship between cytotoxicity and the composition of heterogeneous samples. PD derived aggregates were mixed with αS fibrils keeping the overall monomer-equivalent concentration at 5 μM, and were used in an LDH assay to characterize each samples cytotoxicity. (right) LDH assays were also performed on cells incubated with increasing concentrations of fibrils alone (1, 5, 10 μM) as a comparison to the cytotoxicity levels in B.

FIG. 29. AT630 enables SMLM imaging of aggregates in App^{NL-G-F} mouse brain tissues and human brain-derived aggregates. (A) Tissues are stained with AT630 (top left), anti-AB antibody 6E10 (top right), the merged image detected by diffraction-limited microscopy (bottom left) and the same merged image is shown super-resolved (bottom right). Scale bars represent 5 μm. (B) Typical aggregates detected in A are shown. Scale bars represent 500 nm. (C) Aggregates isolated from the PD1 sample were imaged on a coverslip stained with both AT630 (top left) and anti-aS antibodies (top right), with the merged image (bottom left) super-resolved (bottom right). Scale bars represent 5 μm. (D) Typical aggregates from C are shown. Scale bars represent 500 nm.

FIG. 30. Comparison between azimuthal or “spinning”—and conventional TIRF. (A) Four images taken from 0°, 90°, 180° and 270° azimuthal angles of the same field-of-view of a 10 μm mouse brain tissue section under HILO imaging. (B) Average intensity from the four images from A. Scale bars=10 μm. (C) Fluorescence intensity profile plotted from the dashed line in B for (top) each of the four images in A and (bottom) the averaged image in B, showing background suppression with spinning TIRF.

FIG. 31. An example of how amyloid fibrils (protein structures linked to diseases like Alzheimer's) form. The process of fibril formation is typically characterized by three distinct phases: the lag phase, the growth phase, and the plateau phase. Lag phase: monomeric protein units come together to form small clusters known as oligomers. Growth phase: characterized by rapid increase in fibril formation. Plateau phase: the fibril concentration in the solution remains relatively constant.

FIG. 32. An exemplary convolutional autoencoder (CAE) architecture. In this example the encoder compresses the input images to a 14-dimensional latent space. The decoder reconstructs the input from the latent features. The weights of the fully connected neural network are the input to the clustering layer.

FIG. 33. Mean Squared Error (MSE) of a convolutional autoencoder trained with varying embedding sizes (4, 8, 16, 32, 64, 128, 256, and 512). The graph reveals the trend in the MSE across different embedding dimensions, with the 64-embedded value embedding size exhibiting the lowest MSE.

FIG. 34. Comparison of the autoencoder's reconstruction error measured by the mean squared error (MSE) on aggregates from the brain and Cerebrospinal Fluid (CSF) regions.

FIG. 35. Examples of images reconstructed using the convolutional autoencoder.

FIG. 36. Principal Component Variance Plot. PCA transformation on the brain test data. The first principal component captures 55% of the total variance, while the cumulative variance of the first five principal components accounts for approximately 70% of the overall variance.

FIG. 37. Scatter plots illustrating the relationships between the principal components derived from the data corresponding to the brain samples. The left-hand plot depicts PC1 vs PC2, while the right-hand plot shows PC2 vs PC3. Each datapoint is labeled based on disease class: Alzheimer's Disease (AD), Parkinson's Disease (PD), Frontotemporal Dementia (FTD), Dementia with Lewy Bodies (DLB), and Control.

FIG. 38. Scatter plots illustrating the relationships between the principal components derived from the data corresponding to the CSF samples. The left plot depicts PC1 vs PC2, while the right plot shows PC2 vs PC3. Each datapoint is color-labeled based on disease class: Alzheimer's Disease (AD), Parkinson's Disease (PD), and Control.

FIG. 39. A scatter plot illustrating the relationship between PC1 and PC2. By tracing back specific data points to the original images, visual patterns emerge in aggregate shapes. Moving from left to right in the scatter plot is associated with changes in the aggregate shape, as is the transition from top to bottom. These images provide a tangible context to the principal component dispersion.

FIG. 40. Heatmap displaying the correlations among the first ten Principal Components for the brain samples. Only PC1 demonstrates highly significant correlations. While most PCs exhibit negative correlations, PC5 stands out as the exception with a positive correlation.

FIG. 41. Heatmap displaying the correlations among the first ten Principal Components for the CSF samples. The Principal Components for the CSF samples show similar correlations as for those of the brain samples illustrated in FIG. 40.

FIG. 42. An illustration of the relationship between principal components and shape features of aggregates/disease classes for brain samples.

FIG. 43. An illustration of the relationship between principal components and shape features of aggregates/disease classes for CSF samples.

FIG. 44. Density map highlighting disease-specific aggregates. Despite variations in the aggregates for each disease, the spatial location identifying each disease remains consistent. The position of an aggregate on the map can thus be used as an indicator to distinguish its associated disease.

FIG. 45. Differential density map comparing AD and control groups in principal component (PC) space for brain soak samples.

FIG. 46. Differential density map comparing FTD and control groups in PC space for brain soak samples.

FIG. 47. Differential density map comparing DLB and control groups in PC space for brain soak samples.

FIG. 48. Differential density map comparing PD and control groups in PC space for brain soak samples.

FIG. 49. Differential density map comparing AD and control groups in PC space for CSF samples.

FIG. 50. Density plot indicating the regions of highest concentration or intensity for the disease aggregate for AD for the CSF samples.

FIG. 51. Differential density map comparing PD and control groups in PC space for CSF samples.

FIG. 52. Density plot indicating the regions of highest concentration or intensity for the disease aggregate, for PD for the CSF samples.

### DETAILED DESCRIPTION OF THE INVENTION

The accumulation of proteins into aggregates is a common feature of proteopathies. However, protein aggregation is a natural phenomenon, and current diagnostic methods cannot distinguish between normal physiological aggregate species found in healthy individuals and pathological aggregate species found in patients with a proteopathy, or between aggregates associated with different proteopathies.

The inventors have overcome this challenge by identifying a specific sub-population of protein aggregates that are associated with cytotoxicity (also referred to herein as toxicity). This toxic protein aggregate population can be used to not only identify the presence of a proteopathy but also to distinguish between different proteopathies, even when said proteopathies involve aggregation of the same type of protein. The toxic protein aggregates identified by the invention can also be used to assess the severity, stage and/or prognosis of proteopathies, and to monitor the progression of proteopathies.

Using neurodegenerative proteopathies as a model system, the inventors discovered that small protein aggregates (i.e. protein aggregates up to 1 μm in size) were associated with significantly higher cytotoxicity than larger protein aggregates (i.e. protein aggregates >1 μm in size). Furthermore, the abundance of these small protein aggregates was found to be closely correlated with cytotoxicity. The association between these small protein aggregates and cytotoxicity indicates that detection of protein aggregates up to 1 μm in size can be used to screen for the presence of proteopathies.

The inventors have shown for the first time that proteopathy-associated small protein aggregates are morphologically distinct from small protein aggregates present in samples obtained from healthy subjects. Surprisingly, the inventors also discovered that small protein aggregates formed from the same protein, but originating from different proteopathies, exhibit distinct morphologies, thereby enabling the differential diagnosis of proteopathies based on aggregate morphology.

Advantageously, methods of the invention based on determining and comparing the morphology of small protein aggregates can be performed using a single protein aggregate isolated from a patient sample. This is particularly advantageous for early detection based screening methods, e.g. pre-symptomatic screening methods, where samples may contain a low concentration of small protein aggregates.

Small protein aggregates were also found to differ in toxicity depending on the proteopathy with which they are associated. For example, small alpha-synuclein (aS) aggregates from Parkinson's Disease (PD) were found to exhibit higher potency in cytotoxicity assays when compared to small aS aggregates from dementia with Lewy bodies (DLB). These results indicate that differential diagnosis of proteopathies may be achieved by determining the toxicity profiles of protein aggregates isolated from a patient sample. Without wishing to be bound by theory, the inventors believe that the distinct morphologies exhibited by protein aggregates from different proteopathies account for the different toxicity profiles produced by these aggregates.

The invention provides a method of screening for the presence of a proteopathy or an increased risk thereof in a patient, the method comprising: (a) comparing the morphology, toxicity and/or abundance of protein aggregates in a patient sample to a control; and (b) determining if the patient has a proteopathy or an increased risk thereof based on the comparison performed in step (a); wherein the protein aggregates are up to 1 μm in size.

The invention also provides a method of diagnosing a proteopathy in a patient, the method comprising comparing the morphology, toxicity and/or abundance of protein aggregates in a patient sample to a control associated with a distinct proteopathy, wherein: (i) a difference in the morphology, toxicity and/or abundance of protein aggregates between the patient sample and the control indicates the absence of the proteopathy associated with that control; or (ii) no difference in the morphology, toxicity and/or abundance of protein aggregates between the patient sample and the control indicates the presence of the proteopathy associated with that control; wherein the protein aggregates are up to 1 μm in size.

In some embodiments, the control is the morphology, toxicity and/or abundance of small protein aggregates which are associated with a proteopathy. In some embodiments, the control is the morphology, toxicity and/or abundance of small protein aggregates which are not associated with a proteopathy.

In some embodiments, methods of the invention comprise assigning a likelihood that the patient has a proteopathy or an increased risk thereof. For example, if the comparison step indicates that the small protein aggregates in the sample obtained from the patient: (a) exhibit proteopathy-associated morphology, toxicity and/or abundance; and/or (b) do not exhibit non-proteopathy-associated morphology, toxicity and/or abundance, the method may comprise assigning a high likelihood that said subject has a proteopathy. Alternatively, if the comparison step indicates that the small protein aggregates in the sample obtained from the patient: (a) exhibit non-proteopathy-associated morphology, toxicity and/or abundance; and/or (b) do not exhibit proteopathy-associated morphology, toxicity and/or abundance, the method may comprise assigning a low likelihood that said subject has a proteopathy.

In some embodiments, the method of the invention comprises screening for the presence of an increased risk of a proteopathy in a patient. In some embodiments, patients who are identified as being at an increased risk of a proteopathy do not exhibit symptoms associated with a proteopathy. Patients identified as being at an increased risk of a proteopathy are more likely to develop a proteopathy in the future than patients having small protein aggregates exhibiting non-proteopathy-associated morphology, toxicity and/or abundance.

In some embodiments, the method comprises monitoring patients who are identified as being at an increased risk of developing a proteopathy. In some embodiments, monitoring patients identified as being at an increased risk of developing a proteopathy comprises repeating the method of the invention at specified time intervals, e.g. every 6 months, every year, or every two years.

In some embodiments, the methods of the invention comprise screening for a specific proteopathy. In some embodiments, the methods of the invention comprise screening for presence of one of a group of proteopathies wherein each proteopathy in said group comprises aggregation of the same protein. In some embodiments, the group of proteopathies comprises synucleinopathies.

In some embodiments, diagnosis comprises differential diagnosis. As used herein, “differential diagnosis” refers to the determination of which of two or more proteopathies a patient has, or is at risk of developing.

In some embodiments, the method comprises comparing the morphology, toxicity and/or abundance of protein aggregates in a patient sample to a range of controls, wherein each control is associated with distinct proteopathy and differentially diagnosing the proteopathy comprises identifying the control to which the morphology, toxicity and/or abundance of the protein aggregates in the patient sample is most closely associated.

In some embodiments, the diagnosis methods of the invention comprise differentially diagnosing proteopathies involved aggregation of the same protein, such as synucleinopathies.

The invention also provides a method of assessing the severity, stage and/or prognosis of a proteopathy in a patient, the method comprising: (a) comparing the morphology, toxicity and/or abundance of the protein aggregates in the patient sample to one or more control(s), wherein the one or more control(s) is associated with a known severity, stage and/or prognosis of proteopathy; and (b) determining the severity, stage and/or prognosis of the proteopathy based on the comparison performed in step (a); wherein the protein aggregates are up to 1 μm in size.

As used herein, the prognosis of a proteopathy may include predicting the clinical outcome of the patient and/or assessing the risk of progression. Proteopathies having a higher severity and/or later stage have a worse prognosis. Thus, methods of assessing severity and/or stage of proteopathy can be used to determine the prognosis of a proteopathy.

In some embodiment, assessing the prognosis of a proteopathy comprises predicting the timeline of disease, e.g. the rate of progression. In some embodiments, the method further comprises assessing the symptoms experienced by the patient and/or predicting the symptoms the patient may be likely to experience.

In the context of neurodegenerative diseases, currently the most reliable methods for determining disease severity and/or stage involve post-mortem examination of brain tissue, wherein the location of aggregates in different brain regions is used to determine disease progression. Advantageously, the methods of the invention enable accurate diagnosis of disease severity, stage and/or prognosis using a sample obtained from the patient, such as a cerebrospinal fluid sample, a blood sample, a biopsy, or a faecal sample, thereby enabling disease severity, stage and/or prognosis to be determined and monitored in living patients.

In some embodiments, a difference in the morphology, toxicity and/or abundance of small protein aggregates between the patient sample and the control indicates that the proteopathy has a different severity, stage and/or prognosis than the severity, stage and/or prognosis associated with that control.

In some embodiments, the method comprises comparing the morphology, toxicity and/or abundance of the protein aggregates in the patient sample to a range of controls, wherein each control is associated with a known severity, stage and/or prognosis of proteopathy and determining the severity, stage and/or prognosis of the proteopathy by identifying the control to which the morphology, toxicity and/or abundance of the protein aggregates in the patient sample is most closely associated.

Methods for defining disease severity and stage will depend on the particular proteopathy, or class of proteopathies, in question. For example, proteopathies may have specific stages which are referred to e.g. as “Stage 1”, “Stage 2”, etc. In the methods of the invention, the severity, stage and/or prognosis of the proteopathy associated with a particular control is determined using methods known in the art for that particular proteopathy.

The invention also provides a method for monitoring the progression of a proteopathy in a patient, the method comprising: (a) comparing: (i) the morphology, toxicity and/or abundance of the protein aggregates in a patient sample obtained at a first time point with (ii) the morphology, toxicity and/or abundance of the protein aggregates in a patient sample obtained at a second subsequent time point; and (b) determining whether the proteopathy has progressed between the first and second time point based on the comparison performed in step (a); wherein the protein aggregates are up to 1 μm in size.

In some embodiments, a difference in the morphology, toxicity and/or abundance of small protein aggregates between the first time point and the second time point typically indicates that the proteopathy has progressed, whereas no difference in the morphology, toxicity and/or abundance of small protein aggregates between the first time point and the second time point typically indicates that the proteopathy has not progressed. In some embodiments, the method comprises comparing the morphology, toxicity and/or abundance of the small protein aggregates at multiple time points to track the progression of the proteopathy over time.

In some embodiments, the method comprises monitoring the prognosis of the proteopathy in the patient, wherein a difference in the morphology, toxicity and/or abundance of small protein aggregates between the first time point and the second time point typically indicates that the prognosis has worsened between the first and second time points, whereas no difference in the morphology, toxicity and/or abundance of small protein aggregates between the first time point and the second time point typically indicates prognosis has not changed between the first and second time points.

Methods for monitoring the progression of a proteopathy may also comprise comparing the morphology, toxicity and/or abundance of the small protein aggregates at the first and second time points to one or more control(s), wherein each control is associated with a known severity, stage and/or prognosis of proteopathy.

The invention also provides a method for determining the efficacy of a therapeutic intervention in a patient having a proteopathy, the method comprising: (a) comparing: (i) the morphology, toxicity, and/or abundance of protein aggregates in a patient sample obtained prior to a therapeutic intervention with (ii) the morphology, toxicity, and/or abundance of protein aggregates in a patient sample obtained after the therapeutic intervention; and (b) determining the efficacy of the therapeutic intervention based on the comparison performed in step (a); wherein the protein aggregates are up to 1 μm in size.

In some embodiments, a difference in the morphology, toxicity and/or abundance of small protein aggregates between the first and second time points indicates that the therapeutic intervention is efficacious. For example, a lower abundance of small protein aggregates at the second time as compared to the first time point may indicate that the therapeutic intervention has high efficacy. In some embodiments, no difference in the morphology, toxicity and/or abundance of small protein aggregates between the first and second time points indicates that the therapeutic intervention has low or no efficacy.

Methods for determining the efficacy of a therapeutic intervention may also comprise comparing the morphology, toxicity and/or abundance of small protein aggregates at the first and second time points to one or more control(s), wherein each control is associated with a known severity, stage and/or prognosis of proteopathy. In some embodiments, the therapeutic intervention is identified as having high efficacy if the morphology, toxicity and/or abundance of small protein aggregates at the first time point is associated with a higher severity, later stage and/or worse prognosis of proteopathy than the morphology, toxicity and/or abundance of small protein aggregates at the second time point. In some embodiments, the therapeutic intervention is identified as having low or no efficacy if the morphology, toxicity and/or abundance of small protein aggregates at the second time point is associated with the same severity, stage and/or prognosis or a higher severity, later stage and/or worse prognosis of proteopathy than the morphology, toxicity and/or abundance of small protein aggregates at the first time point.

The skilled person will readily understand that the therapeutic intervention will depend on the particular proteopathy in question. In some embodiments, the therapeutic intervention comprises administration of a therapeutic agent to the patient. In some embodiments, the therapeutic intervention comprises administration of an invasive or non-invasive stimulus, such as electric or acoustic stimulation. In some embodiments, the therapeutic intervention has been approved for treatment of a proteopathy. In some embodiment, the therapeutic intervention is predicted to be useful in the treatment of a proteopathy, e.g. in in vitro or in animal models of said proteopathy.

In some embodiments, the therapeutic intervention is an antibody or a macromolecule configured to recognise protein aggregates.

Unless indicated otherwise, references herein to the “methods of the invention” should be understood to include methods of screening for the presence of a proteopathy or an increased risk thereof, methods of diagnosing a proteopathy or an increased risk thereof, methods of differentially diagnosing a proteopathy or an increased risk thereof, methods of assessing the severity, stage and/or prognosis of a proteopathy, methods for monitoring progression of a proteopathy, and methods for monitoring the efficacy of a therapeutic intervention in a patient having a proteopathy.

It will be readily understood that references below to comparison of morphology, toxicity, and/or abundance of small protein aggregates in the patient sample “to a control” embraces comparison of the morphology, toxicity, and/or abundance of small protein aggregates in the patient sample to one or more controls.

As used herein, the size of a protein aggregate is determined by measuring the longest distance that can be measured within a protein aggregate.

As used herein, the term “aggregate” embraces all forms of associated protein entities that may be present during protein aggregation, including, but not limited to, oligomers, protofibrils, filamentous aggregates (fibrils), amorphous aggregates, tangles, and plaques. The term aggregate also embraces Lewy bodies.

The terms “small protein aggregates”, “small aggregates”, “toxic protein aggregates”, and “toxic aggregates” are used interchangeably herein to refer to protein aggregates up to 1 μm in size. In some embodiments, the small protein aggregates of the invention comprise protein aggregates up to 900 nm, up to 800 nm, up to 700 nm, up to 600 nm, up to 550 nm, up to 540 nm, up to 530 nm, up to 520 nm, up to 510 nm, up to 500 nm, up to 490 nm, up to 480 nm, up to 470 nm, up to 460 nm, or up to 450 nm in size. In some embodiments, the small protein aggregates of the invention comprise protein aggregates 450+60 nm in size.

In some embodiments, the methods of the invention comprise detecting protein aggregates up to 900 nm, up to 800 nm, up to 700 nm, up to 600 nm, up to 550 nm, up to 540 nm, up to 530 nm, up to 520 nm, up to 510 nm, up to 500 nm, up to 490 nm, up to 480 nm, up to 470 nm, up to 460 nm, or up to 450 nm in size in the sample obtained from the patient.

In some embodiments, the methods of the invention comprise detecting protein aggregates that are up to 450+60 nm in size in the sample obtained from the patient.

As used herein, “proteopathy-associated small protein aggregates” are small protein aggregates from a sample obtained from a patient having a proteopathy, or from multiple samples obtained from a population of patients having a proteopathy. Said population of patients may have the same proteopathy or proteopathies involving aggregation of the same type of protein aggregate, e.g. synucleinopathies. As used herein, “proteopathy-associated morphology, toxicity and/or abundance” refers to the morphology, toxicity and/or abundance of small protein aggregates which are associated with a proteopathy.

As used herein, “non-proteopathy-associated small protein aggregates” are small protein aggregates from a sample obtained from a patient who does not have proteopathy, or from multiple samples obtained from a population of patients who do not have a proteopathy. Patients who do not have a proteopathy typically refers to patients who have not been diagnosed as having a proteopathy and/or do not exhibit symptoms associated with a proteopathy. As used herein, “non-proteopathy-associated morphology, toxicity and/or abundance” refers to the morphology, toxicity and/or abundance of small protein aggregates which are not associated with a proteopathy.

The skilled person can readily identify suitable controls for use in the methods of the invention. Suitable controls typically include the morphology, toxicity and/or abundance of proteopathy-associated small protein aggregates, and/or the morphology, toxicity and/or abundance of non-proteopathy-associated small protein aggregates. The control may be a threshold value selected to distinguish between small protein aggregates, e.g. between proteopathy-associated small protein aggregates and non-proteopathy-associated small protein aggregates, or between small protein aggregates associated with distinct proteopathies.

The methods of the invention may employ statistical analyses to compare the morphology, toxicity and/or abundance of small protein aggregates to the control or to the morphology, toxicity and/or abundance of small protein aggregates from a different time point. Suitable statistical analyses include, but are not limited to, neural networks, random forest models, and logistic regression. These methods can produce probability values representing the degree to which a subject belongs to one classification out of a plurality of classifications.

In some embodiments, the patient has not previously been diagnosed with a proteopathy. In some embodiments, the patient is suspected of having a proteopathy based on known diagnostic methods, e.g. symptoms analysis or medical imaging (such as PET/SPECT scans). In some embodiments, the patient exhibits proteopathy-associated symptoms. In some embodiments, the patient has been diagnosed with a proteopathy.

In differential diagnosis methods of the invention, the patient may be known to have or be suspected of having a proteopathy, e.g. based on screening methods of the invention or diagnostic methods known in the art (such as PET or SPECT scans or symptom analysis or familial gene mutations of pathogenic nature). Alternatively, the patient may have been identified as being at an increased risk of developing a proteopathy.

Advantageously, the small protein aggregates of the invention can traverse cell membranes and/or the blood-brain barrier, thereby allowing them to be detected in bodily fluid samples. Samples suitable for use in the methods of the invention include, but are not limited to, cerebrospinal fluid (CSF), blood, plasma, serum, or urine. In some embodiments, the sample is a CSF sample. In some embodiments, the sample is a blood, plasma, or serum sample. In some embodiments, the sample is a faecal or biopsy sample.

The sample is typically a sample which has been obtained from the patient, and the method is typically performed ex vivo or in vitro.

In some embodiments, the invention is performed in vivo (e.g. the sample is analysed in situ).

As used herein, the term “proteopathy” typically refers to diseases and conditions associated with the formation of protein aggregates.

In some embodiments, the proteopathy is a neurodegenerative disease. In these embodiments, the methods of the invention typically comprise detecting small amyloid-beta (AB) aggregates, small alpha-synuclein (aS) aggregates, and/or small tau aggregates in the sample obtained from the patient. In some embodiments, the neurodegenerative disease is dementia. Dementia refers to a set of neurodegenerative diseases that commonly affect memory, mood, behaviour, and motor function. Several forms of dementia are associated with protein aggregation. For example, dementia with Lewy bodies (DLB) and Parkinson's disease (PD) are associated with the misfolding and aggregation of alpha-synuclein (as), whereas Alzheimer's disease (AD) and frontotemporal dementia (FTD) are associated with the aggregation of other proteins such as amyloid-beta (AB) and tau.

In some embodiments, the proteopathy is selected from Dementia with Lewy bodies, Parkinson's disease, Alzheimer's disease, frontotemporal dementia, vascular dementia, Creutzfeldt-Jakob disease, amyloidosis, Huntington's disease, Amyotrophic lateral sclerosis (ALS), and prion disease, In some embodiments, the prion disease is bovine spongiform encephalopathy (mad cow disease).

In some embodiments, the proteopathy is a synucleinopathy. Synucleinopathies (also known as a-synucleinopathies) are neurogenerative diseases characterised by the formation of αS aggregates. Synucleinopathies include Parkinson's disease (PD), dementia with Lewy bodies (DLB) and multiple system atrophy. It is difficult to differentiate between patients having distinct synucleinopathies, particularly PD and DLB, based on currently available diagnostic methods because the physical symptoms associated with these diseases typically overlap. Diagnostic methods based on the presence or abundance of αS aggregates are hampered by the presence of αS aggregates in both healthy and diseased subjects. The inventors have shown for the first time that small αS aggregates in particular differ between healthy and diseased subjects, and between patients suffering from distinct synucleinopathies. The findings described herein indicate that this particular sub-population of as aggregates (i.e. small αS aggregates) has diagnostic potential.

Protein aggregation has been shown to play a role in various diseases including cancer, diabetes, cardiovascular disease, and protein aggregate myopathies. In the context of cancer, previous research has indicated that the aggregation of p53 may play a crucial role in carcinogenesis, tumour progression, and the response of cancer cells to apoptotic signals. The accumulation of p53 aggregates in cancer is associated with uncontrolled cell growth. Thus, in some embodiments, the proteopathy is cancer. In some embodiments, the small protein aggregates comprise p53 aggregates.

Research has also shown that the formation of islet amyloid polypeptide (IAPP) aggregates may contribute to β-cell dysfunction. Thus, in some embodiments, the proteopathy is diabetes. In some embodiments, the small protein aggregates comprise IAPP aggregates.

Protein aggregation has also been shown to play a role in cardiovascular disease whereby protein aggregation is associated with cardiac myocyte death and heart failure. Thus, in some embodiments, the proteopathy is cardiovascular disease.

Cystic fibrosis (CF) is a genetic disorder which primarily affects the lungs. Aggregation of the CF transmembrane conductance regulator (CFTR) protein has been demonstrated in the lungs of patients suffering from CF, and in ex-vivo primary cultures of bronchial epithelial cells from CF donors, but not in normal control lungs. Thus, in some embodiments, the proteopathy is cystic fibrosis. In some embodiments, the small protein aggregates comprise CFTR aggregates.

In some embodiments, the proteopathy is a protein aggregate myopathy. Protein aggregate myopathies define muscle disorders characterized by protein accumulation in muscle fibres. In some embodiments, the protein aggregate myopathy is selected from desmin-related myopathy, an inclusion body myopathy, an actinopathy and a myosinopathy.

In some embodiments, the protein aggregate is an htt aggregate, an aggregate containing protein or proteins with tandem glutamine expansions, an TDP-43 aggregate, a prion aggregate, a FUS aggregate, a C9ORF72 aggregate, a ubiquilin-2 aggregate and/or a SOD1 aggregate.

In some embodiments, the protein aggregate is an aggregate assembled from an intrinsically disordered protein, a misfolded protein or any protein that lacks a defined or ordered three-dimensional structure.

In some embodiments, the protein aggregate is post-translationally modified by or otherwise interacting with a ubiquitin protein, a SUMO protein, a phosphate group, an acetyl group, a methyl group, a sugar such as an O-GlcNAc group, a glycosyl group, a nucleic acid group and/or a lipid group.

In some embodiments, the proteopathy is selected from amyloidosis. In some embodiments, the protein aggregates is selected from an immunoglobin aggregate, an Amyloid A protein aggregate, an aggregate containing a member of apolipoproteins, a gelsolin aggregate, a lysozyme aggregate, a fibrinogen aggregate, a microglobulin aggregate, a transthyretin aggregate, a keratin aggregate, a lactoferrin aggregate a corneodesmosin aggregate and an enfuvirtide aggregate.

As described herein, the inventors have discovered that proteopathy-associated small protein aggregates are morphologically distinct from small protein aggregates present in samples obtained from healthy subjects. In addition, the inventors have shown that proteopathies can be differentiated based on the morphology of small protein aggregates. Thus, in some embodiments, the methods of the invention comprise comparing the morphology of small protein aggregates in the patient sample to a control.

Morphological features for use in the methods of the invention include those that can distinguish between: (a) proteopathy-associated small protein aggregates and non-proteopathy-associated small protein aggregates; and/or (b) small protein aggregates associated with distinct proteopathies. The skilled person can readily identify suitable morphological features by comparing the morphology of proteopathy-associated small protein aggregates with non-proteopathy-associated small protein aggregates, or by comparing the morphologies of small protein aggregates from distinct proteopathies.

In some embodiments, morphological features for use in the methods of the invention are identified using pattern recognition methods. Pattern recognition methods may include the use of multivariate statistics to analyse images of small protein aggregates and to classify aggregates. Pattern recognition methods may be unsupervised or supervised. Unsupervised methods typically reduce data complexity in a rational way and produce plots that can be visually interpreted. Unsupervised pattern recognition methods include, but are not limited to, principal component analysis (PCA), hierarchical cluster analysis (HCA), and non-linear mapping (NLM). Supervised methods typically use a training set of characterised data to produce a mathematical model that can then be evaluated with independent validation data sets. Supervised pattern recognition methods include neural networks.

In some embodiments, the morphological features of protein aggregates are identified and quantified using microscopy and image analysis methods. Suitable microscopy and image analysis methods are described in more detail below.

In some embodiments, the protein aggregates have been stained with an aggregate-specific stain, e.g. an aggregate-specific dye as described herein. In some embodiments, the protein aggregates have been stained with an antibody-based label as described herein. In some embodiments, the protein aggregates have been stained with more than one aggregate-specific and/or antibody-based labels. In some embodiments, the morphological features of the protein aggregates may be identified and quantified using multi-channel fluorescence microscopy. In some embodiments, the morphological features of the protein aggregates may be identified and quantified from multicolour images obtained by multi-channel fluorescence microscopy.

In some embodiments, the methods of the invention comprise comparing the area of one or more small protein aggregate(s) in the patient sample to a control. The area of an aggregate may be determined from an image of the aggregate by counting the total number of pixels inside the aggregate. In some embodiments, determining the area of a protein aggregate comprises detecting the fluorescence intensity of each pixel inside an aggregate.

In some embodiments, the methods of the invention comprise comparing the eccentricity of one or more small protein aggregate(s) in the patient sample to a control. As used herein, eccentricity is a measure of ellipticity and is the ratio of the distance between focal points over the major axis when fitting an ellipse to the aggregate.

In some embodiments, the methods of the invention comprise comparing the solidity of one or more small protein aggregate(s) in the patient sample to a control. The solidity of an aggregate may be determined from an image of the aggregate by calculating the proportion of pixels inside a convex hull fitted to an aggregate that are also part of the aggregate.

In some embodiments, the methods of the invention comprise comparing the skeleton size of one or more small protein aggregate(s) in the patient sample to a control. The skeleton size of an aggregate may be determined from an image of the aggregate by calculating the sum of the pixels in the skeletonized aggregate, where skeletonisation iteratively removes border pixels whilst maintaining connectivity (see e.g. Tongjie Y Zhang and Ching Y. Suen. Communications of the ACM, 27(3):236-239, 1984).

In some embodiments, the methods of the invention comprise comparing the number of branches of one or more small protein aggregate(s) in the patient sample to a control. The number of branches may be determined from an image of the aggregate by calculating the number of branching points in the skeletonized image.

In some embodiments, the method comprises comparing two or more of the area, eccentricity, solidity, skeleton size, and number of branches of one or more small protein aggregate(s) in the patient sample to a control. In some embodiments, the method comprises comparing three or more of the area, eccentricity, solidity, skeleton size, and number of branches of one or more small protein aggregate(s) in the patient sample to a control. In some embodiments, the method comprises comparing four or more of the area, eccentricity, solidity, skeleton size, and number of branches of one or more small protein aggregate(s) in the patient sample to a control. In some embodiments, the method comprises comparing the area, eccentricity, solidity, skeleton size, and number of branches of one or more small protein aggregate(s) in the patient sample to a control. In some embodiments, the method comprises comparing the area, eccentricity, and solidity of one or more small protein aggregate(s) in the patient sample to a control.

In some embodiments, the method comprises comparing one or more of area, solidity, skeleton size, and number of branches of one or more small protein aggregate(s) in the patient sample to a control. In some embodiments, the method comprises comparing area, solidity, skeleton size, and number of branches of one or more small protein aggregate(s) in the patient sample to a control.

In some embodiments, the method comprises comparing the area and eccentricity of one or more small protein aggregate(s) in the patient sample to a control. In some embodiments, the method comprises comparing the area and solidity of one or more small protein aggregate(s) in the patient sample to a control. In some embodiments, the method comprises comparing the area and skeleton size of one or more small protein aggregate(s) in the patient sample to a control. In some embodiments, the method comprises comparing the area and number of branches of one or more small protein aggregate(s) in the patient sample to a control. In some embodiments, the method comprises comparing the eccentricity and solidity of one or more small protein aggregate(s) in the patient sample to a control. In some embodiments, the method comprises comparing the eccentricity and skeleton size of one or more small protein aggregate(s) in the patient sample to a control. In some embodiments, the method comprises comparing the eccentricity and number of branches of one or more small protein aggregate(s) in the patient sample to a control. In some embodiments, the method comprises comparing the solidity and skeleton size of one or more small protein aggregate(s) in the patient sample to a control. In some embodiments, the method comprises comparing the solidity and number of branches of one or more small protein aggregate(s) in the patient sample to a control. In some embodiments, the method comprises comparing the skeleton size and number of branches of one or more small protein aggregate(s) in the patient sample to a control.

In some embodiments, the control comprises the area, eccentricity, solidity, skeleton size, and/or number of branches of proteopathy-associated small protein aggregates. In some embodiments, the control may be the area, eccentricity, solidity, skeleton size, and/or number of branches of non-proteopathy-associated small protein aggregates.

In some embodiments, the morphological features of the protein aggregates comprise one or more of solidity, eccentricity, number of branches, skeleton size, area, perimeter, perimeter of the convex hull, projection area, filled projection area, area of the convex hull, major axis length of the legendre ellipse of inertia (ellipse that has the same normalized second central moments as the particle shape), minor axis length of the legendre ellipse of inertia, diameter of the maximum incircle of the projection area, diameter of the minimum circumference of the projection area, diameter of the circumcircle with the same centre as the particle contour and maximum area which touches the particle contour from the inside, diameter of the circumcircle with the same centre as the particle contour and minimum area which touches the particle contour from the outside, diameter of a circle of equal projection area, diameter of a circle of equal perimeter, maximum longest chord, longest chord orthogonal to the maximum longest chord, width of the minimal 2D bounding box, length of the minimal 2D bounding box, geodetic length of the protein aggregate, thickness of the aggregate, number of pixel erosions to completely erase the silhouette of a particle in a binary image, number of pixel erosions to completely erase the complement between convex hull and object, fractal dimension determined by the box counting method, fractal dimension determined by the perimeter method, maximum ferret diameter of the aggregate (the ferret diameter is a measure of an object's size along a specified direction), minimum ferret diameter of the aggregate, median of all ferret diameters, arithmetic mean of all ferret diameters, standard deviation of all ferret diameters, maximum Martin diameter (the Martin diameter is the length of the area bisector of an irregular object in a specified direction of measurement), minimum Martin diameter, median of all Martin diameters, median of all Martin diameters, arithmetic mean of all Martin diameters, standard deviation of all Martin diameters, maximum Nassenstein diameter, minimum Nassenstein diameter, median of all Nassenstein diameters, arithmetic mean of all Nassenstein diameters, standard deviation of all Nassenstein diameters, maximum of max chords (max chord is the maximum of all chords for one particle rotation), minimum of max chords, median of max chords, mean of max chords, standard deviation of max chords, maximum of all chords for all rotations, median of all chords for all rotations, mean of all chords for all rotations, and standard deviation of all chords for all rotations.

Surprisingly, the inventors have discovered that small protein aggregates associated with different neurodegenerative proteopathies are morphologically distinct. In more detail, the inventors have demonstrated that the morphology of small protein aggregates can be used to diagnose (e.g. differentially diagnose) patients suffering from dementia with Lewy bodies (DLB), Parkinson's disease (PD), Alzheimer's disease (AD) and frontotemporal dementia (FTD) with an accuracy of >77%. Thus, in some embodiments, differential diagnosis methods of the invention comprise differentially diagnosing two or more of Dementia with Lewy bodies, Parkinson's disease, Alzheimer's disease and frontotemporal dementia. In some embodiments, differential diagnosis methods of the invention comprise differentially diagnosing Dementia with Lewy bodies, Parkinson's disease, Alzheimer's disease and frontotemporal dementia. In some embodiments, the methods of the invention comprise differentially diagnosing Dementia with Lewy bodies, Parkinson's disease, Alzheimer's disease and frontotemporal dementia based on the area, eccentricity, solidity, skeleton size, and number of branches of small protein aggregates in the patient sample.

Advantageously, the inventors have demonstrated herein that the morphology of small protein aggregates can be used to diagnose (e.g. differentially diagnose) patients suffering from PD from patients suffering from DLB with an accuracy of >88%. In some embodiments, differential diagnosis methods of the invention comprise differentially diagnosing PD and DLB. In some embodiments, methods of the invention comprise differentially diagnosing PD and DLB based on the area, eccentricity, solidity, skeleton size, and number of branches of small protein aggregates in the patient sample.

Importantly, the inventors have demonstrated that specific diseases can be identified based on morphological analysis of aggregates present in the cerebrospinal fluid with an accuracy of >80%. In some embodiments, differential diagnosis methods of the invention comprise differentially diagnosing proteopathies based on morphological features comprising two or more of the area, solidity, skeleton size, eccentricity and number of branches of small protein aggregates in a patient sample. In some embodiments, differential diagnosis methods of the invention comprise differentially diagnosing proteopathies based on morphological features comprising the area, solidity, skeleton size and number of branches of small protein aggregates in a patient sample. In some embodiments, the sample is a CSF sample.

In some embodiments, methods of the invention comprising screening for the presence of a synucleinopathy comprise comparing the area, eccentricity, solidity, skeleton size, and number of branches of small protein aggregates in the patient sample to a control. The inventors have demonstrated herein that synucleinopathy-associated small αS aggregates typically exhibit greater area, eccentricity, skeleton size, and number of branches, and lower solidity than non-proteopathy-associated small αS aggregates.

In some embodiments, the methods of the invention comprise staining the sample obtained from the subject with an aggregate-specific fluorescent dye or a fluorescence labelled antibody specific to a protein prone to form aggregates, and comparing the fluorescence intensity of one or more small protein aggregate(s) in the patient sample to a control. Small protein aggregates exhibit distinct fluorescence intensities depending on their size and/or structure, and so fluorescence intensity can be used to determine and/or compare aggregate morphology.

The inventors have also demonstrated herein that the toxicity of small protein aggregates can depend on the type of proteopathy with which they are associated. Thus, in some embodiments, the methods of the invention comprise comparing the toxicity of the small protein aggregates in a patient sample to a control. The toxicity of small protein aggregates in a patient sample can be used to indicate the presence or absence of a proteopathy, and also to differentiate between distinct proteopathies. As used herein, the “toxicity”, “toxicity profile” or “potency” of a protein aggregate is the relationship between concentration of said aggregate and toxicity. A small protein aggregate which achieves greater cytotoxicity at lower concentration has a higher potency than a small protein aggregate that requires a higher concentration to achieve the same level of cytotoxicity. Toxicity of small protein aggregates may correlate with their ability to penetrate lipid membranes, cause oxidative mitochondrial and other stress, inflammation, DNA damage and to induce further aggregation.

In some embodiments, the toxicity profile of protein aggregates is determined by exposing cells to increasing concentrations of said protein aggregate and calculating the toxicity at each concentration. For example, toxicity may be measured by detecting and/or quantifying cell stress, cell viability, and/or cell cytotoxicity using a lactate dehydrogenase (LDH) assay (see e.g. Kaja, S. et al. J Pharmacol Toxicol Methods 73, 1-6 (2015)), a (2′,7′-dichlorofluorescin diacetate) DCFDA assay, or an Amplex Red assay (ThermoFisher). The potency of a small protein aggregate may be quantified using a mathematical formula, e.g. linear regression.

In some embodiments, the toxicity of small protein aggregates is determined by detecting (e.g. quantifying) the formation of proteasome foci. Proteasome foci describe intracellular entities, structures or bodies with high concentrations of proteasome particles relative to the background cytoplasmic concentration. Proteasome foci may be also referred to, for example, as proteasome storage granules, clastosomes, and proteasome stress granules. As described herein, the inventors have found that the formation of proteasome foci is dependent on the level of toxic aggregates present in a sample. Thus, proteasome foci formation provides an indication of aggregate toxicity. Methods of detecting the formation of proteasome foci are known in the art. For example, as described herein, proteasome foci formation can be detected by contacting cells expressing a labelled proteasome (e.g. cells expressing a proteasome subunit labelled with a fluorescent dye, such as GFP) with small protein aggregates, and imaging the cells using fluorescent microscopy to detect proteasome foci formation.

In the context of neurodegenerative proteopathies, the inventors have shown that small protein aggregates from Parkinson's Disease (PD) exhibit different, e.g. higher, potency than small protein aggregates from dementia with Lewy bodies (DLB). Thus, differential diagnosis of proteopathies, e.g. synucleinopathies, may be achieved by determining the toxicity profiles of protein aggregates isolated from a patient sample and comparing said toxicity profiles to the toxicity profiles associated with distinct proteopathies, e.g. distinct synucleinopathies.

As demonstrated herein, the abundance of small protein aggregates was found to correlate with toxicity in neurodegenerative proteopathies. Thus, in some embodiments, the methods of the invention comprise comparing the abundance of small protein aggregates in the patient sample to a control. Previous research, in the context of dementia, has suggested that the total abundance of protein aggregates cannot be used to screen for the presence of proteopathies due to the natural variation in protein aggregate abundance in both diseased and healthy subjects. However, the inventors have unexpectedly shown that the abundance of small protein aggregates as defined by the invention correlates with cytotoxicity indicating that the abundance of this sub-population of protein aggregates is indicative of the presence of a proteopathy, or an increased risk thereof.

In some embodiments, methods of the invention comprise assessing the severity, stage and/or prognosis of a proteopathy by comparing the abundance of small protein aggregates in a patient sample to one or more controls. Advantageously, the inventors have found that the abundance of small protein aggregates correlates with toxicity, and that the abundance of small protein aggregates increases during disease progression. Thus, a higher abundance of small protein aggregates is associated with a higher severity, later stage, and/or worse prognosis of proteopathy.

The abundance of small protein aggregates may be determined by calculating the relative abundance of small protein aggregates in a sample, e.g. by measuring the abundance of small protein aggregates relative to the total abundance of protein aggregates in a sample.

In some embodiments, the methods of the invention comprise identifying and quantifying small protein aggregates in the patient sample having a proteopathy-associated morphology, and comparing the abundance of said small protein aggregates having a proteopathy-associated morphology to a control. In such embodiments, a higher abundance of small protein aggregates in the patient sample having a proteopathy-associated morphology as compared to the control typically indicates the presence of a proteopathy, or that the proteopathy has a higher severity, later stage and/or worse prognosis than the severity, stage and/or prognosis of proteopathy associated with that control. Aggregates having a proteopathy-associated morphology may be identified using morphological features that differ between proteopathy-associated small protein aggregates and non-proteopathy-associated small protein aggregates as described above.

In embodiments comprising comparing the abundance of protein aggregates in the patient sample to one or more control(s), wherein each control is associated with a known severity, stage and/or prognosis of proteopathy, a higher abundance of small protein aggregates in the patient sample as compared to the control typically indicates that the patient has a higher severity, later stage and/or worse prognosis of proteopathy than the severity, stage and/or prognosis of proteopathy associated with that control.

In some embodiments, the method of the invention comprises staining the sample obtained from the subject with an aggregate-specific dye, e.g. an aggregate-specific fluorescent dye. In some embodiments, the size and/or structure of protein aggregates is determined using fluorescence intensity. Fluorescence intensity of protein aggregates correlates with the size and/or structure of the aggregate and so, in some embodiments, small protein aggregates as defined herein are identified by comparing the fluorescence intensity of protein aggregates to a control. In some embodiments, the control is a threshold value selected to distinguish between small protein aggregates and protein aggregates having a size of >1 μm.

Several aggregate-specific dyes are available commercially and include, but are not limited to, ProteoStat (available from Enzo Life Sciences) and Amytracker, e.g. Amytracker 630 (available from Ebba Biotech). In some embodiments, the dye is not protein specific. For example, ProteoStat and Amytracker bind to aggregates without being protein specific and can therefore be used to detect protein aggregates from a range of different proteopathies. Fluorophores like Thioflavin S and Thioflavin T (ThT) also enable detection of aggregate structures, ThT reversibly binds β-sheet structures in amyloid assemblies and increases its fluorescence intensity upon binding. Similar aggregate-activated fluorophores such as pFTAA, Nile Red and Th-X have also been developed for aggregate detection using a range of dedicated fluorescence techniques. In some embodiments, the methods of the invention comprise contacting the patient sample with a dye selected from ProteoStat, Amytracker (e.g. Amytracker 630), Thioflavin S, Thioflavin T (ThT), pFTAA, Nile Red, Th-X, and Congo Red. The methods of the invention may comprise antibody-coupled aggregate detection through DNA-PAINT as described e.g. in Lobanova, E. et al. Brain, 145 (2): 632-643; DOI: 10.1093/brain/awab306 (2021).

In some embodiments, the methods of the invention comprise staining protein aggregates using cell membrane permeating dyes. Suitable cell permeating dyes include, but are not limited to, Amytracker. Cell membrane permeating dyes advantageously enable imaging of small, toxic aggregates in cells in situ. It will be readily understood that aggregate staining is not limited to staining with dyes, but embraces any staining method suitable to enable detection of the aggregates.

In some embodiments, the method of the invention comprises staining the sample obtained from the subject with an antibody-based label, e.g. an antibody-based dye. In some embodiments, the method of the invention comprises contacting the sample with an anti-Tau antibody conjugated to a detectable label, e.g. a dye. In some embodiments, the method of the invention comprises contacting the sample with an anti-amyloid-β antibody conjugated to a detectable label, e.g. a dye. In some embodiments, the method of the invention comprises contacting the sample with an anti-a-synuclein antibody conjugated to a detectable label, e.g. a dye. In some embodiments, the method comprises staining the sample obtained from the subject with an aggregate-specific dye and one or more antibody-based labels. In some embodiments, the dye comprises a fluorophore. When a sample is contacted with more than one antibody-based labels, each antibody typically comprises a different label.

In some embodiments, methods of the invention comprise detection of small protein aggregates by high resolution microscopy methods. In some embodiments, methods of the invention comprise detection of small protein aggregates by super resolution microscopy methods. Advantageously, the use of super-resolution microscopy methods provides an accurate picture of the size distribution of aggregates, and is ideally-suited to identification and characterisation of the small protein aggregates present in a sample. In some embodiments, detection of small protein aggregates comprises performing single-molecule localisation microscopy (SMLM), total-internal reflection fluorescence (TIRF) microscopy, Stochastic Optical Reconstruction Microscopy (STORM), Photoactivated Localization Microscopy (PALM), Stimulated Emission Depletion (STED) microscopy, transmission electron microscopy, structured illumination microscopy, light-sheet microscopy, scanning probe microscopy, confocal microscopy, two-photon microscopy, or fluorescence lifetime imaging. SMLM techniques manage to surpass the classical diffraction limit of optical resolution of about half the wavelength of the emitted light. Methods such as STORM, PALM, and STED microscopy employ switchable fluorophores to separate out densely packed foci temporally, enabling them to be imaged at higher resolutions. In some embodiments, detection of small protein aggregates comprises cryogenic electron microscopy (cryo-EM). In some embodiments, detection of small protein aggregates comprises Atomic force microscopy (AFM). In some embodiments wherein the aggregates are stained with one or more fluorescent dyes, the method comprises detection of small protein aggregates by multi-channel fluorescence microscopy.

In some embodiments, detection of small protein aggregates comprises performing SMLM. Advantageously, SMLM approaches are less expensive and less resource intensive than imaging methods such as cryo-EM and AFM whilst achieving the high resolution required to distinguish morphological features of protein aggregates, which increases the potential for clinical scalability.

In some embodiments, the methods of the invention comprise detecting protein aggregates up to 510 nm in size in a sample obtained from the subject using SMLM. In some embodiments, the methods of the invention comprise detecting protein aggregates up to 450±60 nm in size in a sample obtained from the subject using SMLM.

### Classification and Analysis

As described above, the morphology of small protein aggregates differs between proteopathies, even when said proteopathies involves aggregation of the same proteins. Thus, it is possible to classify a proteopathy in a patient by based on the morphology of small protein aggregates in a patient sample. Methods of classifying protein aggregates based on images of the protein aggregates will now be described.

In one example, a convolutional neural network (CNN) is used to classify two common types of dementia (Parkinson's disease (PD) and dementia with Lewy bodies (DLB)), as well as healthy samples, from images of misfolded protein aggregates. In a further example, a random forest method is used to classify the samples. Advantageously, the inventors have realised that morphological differences between the diseases (and the healthy samples) enable the diseases to be classified (e.g. into sub-types of dementia) with high accuracy using the images of the protein aggregates. Moreover, the classification methods are able to classify disease using super-resolution imaging data, which is far less resource intensive than equivalent conventional or “non super-resolution” imaging methods.

Whilst in the examples described below the classification of a proteopathy can be based on an image of a single protein aggregate, it will be appreciated that alternatively many images of aggregates per patient may be obtained and analysed. In this case, the overall classification may correspond, for example, to the most common classification obtained for each protein aggregate.

### Apparatus for Classification

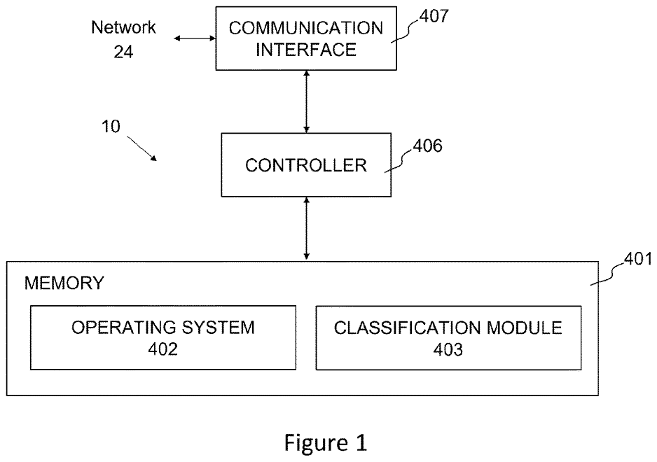

FIG. 1 shows classification apparatus 10 for classifying protein aggregates. It will be appreciated that the classification apparatus may comprise any suitable computer or server. As shown, the classification apparatus 10 includes a communication interface 407 which is operable to transmit signals to and receive signals from other devices via a network 24. For example, the classification apparatus 10 may receive images of protein aggregates via the network 24. Alternatively, the images of the protein aggregates may be loaded from a removable data storage device (RMD), for example.

The classification apparatus may also receive metadata accompanying the images describing the aggregates, including but not limited to, the toxicity of aggregates, information from the patient such as age of onset, progression, sex, and/or clinical dementia rating (CDR) score.

A controller 406 controls the operation of the classification apparatus 10 in accordance with software stored in a memory 401. The software may be pre-installed in the memory 401 and/or may be downloaded via the network 24 or from a removable data storage device (RMD), for example. The software includes, among other things, an operating system 302 and a classification module 403.

The classification module 403 classifies protein aggregates as being associated with a proteopathy, or as being associated with a healthy sample. The classification module 403 may be configured to classify a protein aggregate as being associated with a particular proteopathy. The classification module 403 may be configured to perform any of the classification methods described below. For example, the classification module 403 may be operable to classify samples using a CNN, or using a random forest method. The classification module 403 receives input data corresponding to an image of a protein aggregate, and outputs an indication of whether the protein aggregate is associated with a proteopathy. The classification module 403 may be configured to perform pre-processing of an image of a protein aggregate, and to perform classification using the output of the pre-processing step. As described below, the classification module 403 may analyse input data corresponding to a series of images of protein aggregates from a patient, and output a cytotoxicity score for that patient.

The classification apparatus 10 has been described for ease of understanding as having a number of discrete modules (such as the classification module 403). Whilst these modules may be provided in this way for certain applications, for example where an existing system has been modified to implement the invention, in other applications, for example in systems designed with the inventive features in mind from the outset, these modules may be built into the overall operating system or code and so these modules may not be discernible as discrete entities. These modules may also be implemented in software, hardware, firmware, or a mix of these. As those skilled in the art will appreciate, the software modules may be provided in compiled or un-compiled form and may be supplied to the classification apparatus 10 as a signal over a computer network, or on a recording medium. Further, the functionality performed by part or all of this software may be performed using one or more dedicated hardware circuits. However, the use of software modules is preferred as it facilitates the updating of the classification apparatus 10 in order to update the functionalities.

Each controller may comprise any suitable form of processing circuitry including (but not limited to), for example: one or more hardware implemented computer processors; microprocessors; central processing units (CPUs); arithmetic logic units (ALUs); input/output (IO) circuits; internal memories/caches (program and/or data); processing registers; communication buses (e.g. control, data and/or address buses); direct memory access (DMA) functions; hardware or software implemented counters, pointers and/or timers; and/or the like.

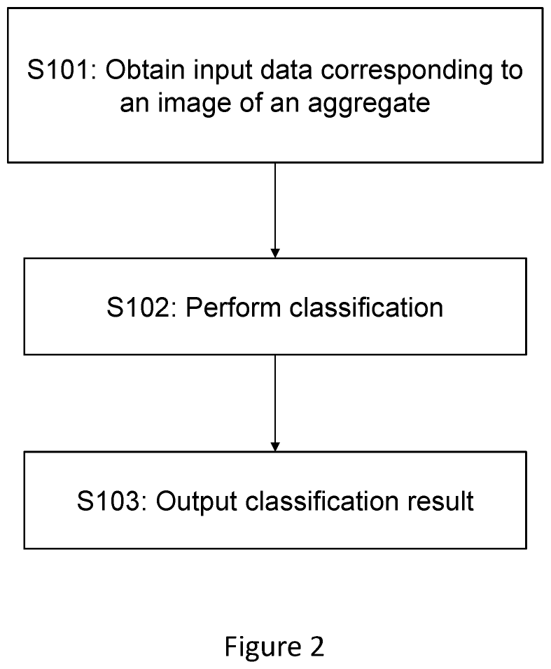

FIG. 2 shows a method of classification that may be performed by the classification apparatus 10. In step S101, input data corresponding to an image of an aggregate is obtained. The input data may be a processed image of a protein aggregate. In step S102, classification is performed using the input data. The classification may comprise classifying the input data as corresponding to a particular type of proteopathy, or may correspond, for example, to a binary classification of ‘healthy’ or ‘diseased’. In step S103 the result of the classification is output. The input data obtained in step S101 may be the input data used in any of the examples described herein, and similarly the classification in step S102 may be performed using any of the corresponding classification methods described herein.

### Image Acquisition

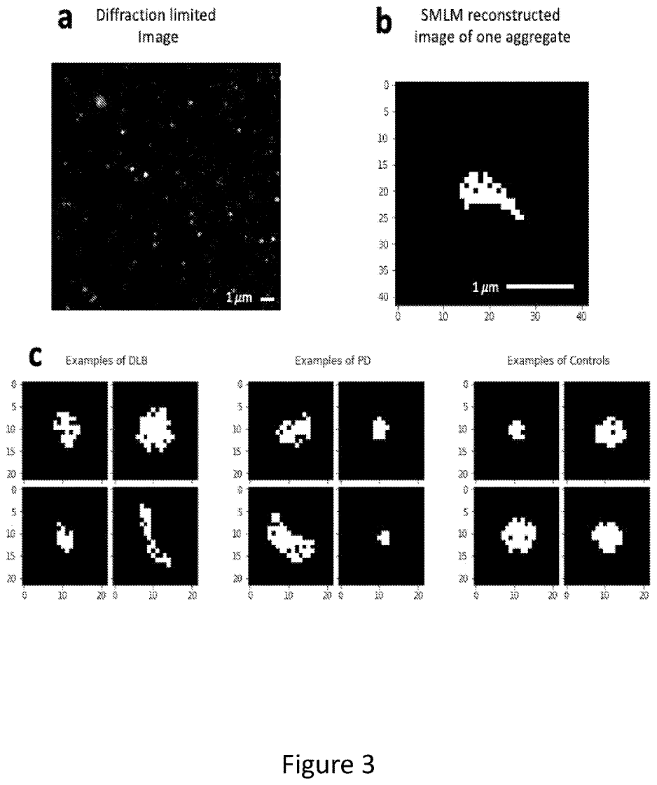

Images used as inputs in the classification methods may be, for example, of the type illustrated in Figures (see e.g. FIGS. 3B, 3C, 12C, 23B, 23D, 26A, and 26D). The protein aggregates may be imaged using any high resolution imaging method as described above. For example, the protein aggregates may be imaged using single-molecule localisation microscopy (SMLM), which provides high resolution imaging. Advantageously, SMLM approaches are less expensive and less resource intensive than cryogenic electron microscopy (cryo-EM) and atomic force microscopy (AFM), whilst achieving a high resolution that enables unique molecular arrangements of misfolded proteins to be distinguished.

Aggregates may be imaged in a SMLM buffer (PBS with 1 mg/mL glucose oxidase, 0.02 mg/ml catalase, 10% (w/w) glucose, and 100 mM methylamine (MEA)) and stained with Amytracker. Images of single aggregates can be obtained using a total-internal reflection fluorescence (TIRF) microscope.

For imaging in live cells, conventional cell media such as Dulbecco's Modified Eagle Medium (DMEM) or fluorobright may be used to keep cells viable whilst imaging is performed.

In this example, the images obtained from the microscope are pre-processed to obtain reconstructed SMLM images. It will be appreciated that any suitable type of pre-processing may be used to prepare the images for analysis and classification. For example, each frame may be filtered with a box filter, with a box four times greater than the width of Gaussian point spread function (PSF). Intensity for each pixel can be weighted by the inverse of its variation. Local maxima can be recorded and Maximum likelihood estimation (MLE) can be used to fit these to a 2-D Gaussian single PSF. A rolling ball filter and a real-space bandpass filter can be applied for background subtraction and to suppress pixel noise and long wavelength image variations. In some embodiments, the processed aggregate images may be combined with the fluorescence intensity information from the pre-processing stage.

In order to obtain images of single aggregates for feature extraction and analysis, a bounding box can be drawn around each individual aggregate (for example, ten pixels beyond the most extreme pixel values corresponding to the aggregate). In order to preserve the size difference between aggregates whilst creating inputs of the same dimensions, images can be padded to the size of the largest aggregate bounding box (e.g. 164×164 pixels). Images of aggregates under four pixels in size may be removed to reduce risk of inclusion of background noise.

### Training Data

Training data for use in training the classification methods described herein may comprise images of aggregates from healthy donors (control samples), and images of aggregates from donors diagnosed with Parkinson's Disease or dementia with Lewy bodies (or any other suitable proteopathy to be classified). Examples of aggregates from dementia with Lewy bodies (DLB), Parkinson's (PD), and healthy samples are shown in FIG. 3C.

### Dimensionality Reduction

In addition to the methods described above, protein aggregate morphologies can also be classified using dimensionality reduction methods, such as principal component analysis (PCA) methods and/or use of an autoencoder, to identify aggregate subpopulations associated with each disease.

### Autoencoders

Autoencoders serve as powerful unsupervised learning algorithms for feature extraction and dimensionality reduction. They are particularly effective in detecting subtle patterns in data, which may be beneficial in the early detection of neurodegenerative diseases, where symptoms can often be minimal and hard to identify. Autoencoders can be used for unsupervised learning tasks such as data compression and noise reduction, and can be adapted for supervised learning by appending a regression or classification layer to their lower-dimensional embedded space. In this adapted setup, the autoencoder first learns to encode the input data in a reduced dimensional space and then uses that encoding for supervised prediction. For the final prediction layer, methods such as linear regression (for regression tasks) or softmax functions (for classification tasks) can be used.

Advantageously, autoencoders work under the principle of reducing data dimensionality by learning a compressed representation of the input (in an ‘embedded space’, which may also be referred to as the ‘latent space’), and then reconstructing the original data from this compressed form. By training to minimize the difference between the input and its reconstruction, autoencoders tend to retain only the most salient features in the lower dimensional embedded space, thereby filtering out noise and unnecessary information. This leads to the generation of stable and robust feature representations.

Autoencoders compress data into a more compact, lower-dimensional form. In doing so, they capture the most relevant features needed to recreate the original input. This process involves two main components: (1) the encoder, which transforms the input into a condensed representation known as the “latent space” or “embedded space”, and (2) the decoder, which tries to rebuild the original input from this compressed form. Convolutional Autoencoders (CAEs) are an extension of autoencoders that leverage convolutional operations (convolutional layers) from convolutional neural networks to extend autoencoders to image analysis. An exemplary convolutional autoencoder (CAE) architecture is illustrated in FIG. 32. As shown in FIG. 32, the autoencoder includes an input layer, a series of lower dimensional encoding layers (Conv1 to Conv 4), and the condensed representation in the centre of the figure. This is followed by a series of decoding layers (ConvT1 to ConvT3) that increase in dimension towards the output layer. Convolutional autoencoders reduce dimensionality and filter out noise, leading to more stable and robust image representations. Convolutional autoencoder models use a neural network structure with weights and biases set for each layer. The learning takes place in an embedded space with chosen dimensionality (e.g., 4, 8, 16, 32, 64, 128, 256) to strike a balance between computational efficiency and capturing essential details. The model undergoes a training process that iteratively refines these weights and biases. The input is propagated through the network using these trained weights and biases, to generate the embedded representation. The discrepancy between the output and the expected result (the input) is computed as an error, which is then propagated backward through the network via backpropagation, facilitating the adjustment and update of the weight parameters. In other words, the convolutional autoencoder model learns by repeatedly passing the input data through the network, calculating the difference between its output and the expected result using a loss function, and then adjusting the parameters of the model based on this error. This continuous refinement helps the model filter noise and produce effective representations in the designated embedding size. Beneficially, principal component analysis (PCA) can be performed using the lower dimensional representation of images of protein aggregates generated using the autoencoder, enabling disease classification (e.g. using nearest neighbour search).

Convolutional autoencoders retain spatial data and are efficient at extracting critical information from noisy, high-dimensional images. They provide a middle ground balancing computational efficiency and detail retention. By integrating convolutional layers, convolutional autoencoders adeptly capture spatial hierarchies and relative positioning within images. Once processed, the lower-dimensional representation from the autoencoder can be further utilized by other convolutional neural networks for tasks like classification (or used as an input for any other suitable method of classification).

### Principal Component Analysis (PCA) and Nearest Neighbour Search (NNS)

Principal Component Analysis (PCA) enables the most variable features in a dataset—often the most important—to be identified, reducing the complexity of the data. Two advantages of PCA in neuroimaging classification methods are ease of use and computational efficiency. Moreover, PCA does not require specific categorical or continuous labels to discern relevant features. Beneficially, PCA enables systematic comparisons across a large dataset of images to be performed. It increases the information's interpretability and aids in determining which aspects of a dataset are the most significant.

In order to retain as much information as possible, a PCA variance plot is used to determine which of the new variables contains the most information. The new variables are based on the dataset and not specified a priori, making PCA an adaptive method of data interpretation. The variables can then be arranged in order of their significance, with the first principal component capturing the maximum variance in the dataset and each subsequent component capturing less variance.

In nearest neighbour search (NNS) methods, given a predetermined database, denoted as X, coupled with a specific measure of dissimilarity, represented by d, and a particular query, labelled as q, the primary objective of NNS is to identify an element x within the database X that has the least dissimilarity or closest proximity to the query q as measured by the dissimilarity function d (x,q). The principle behind NNS aids in locating the most similar or relevant data points from a larger dataset, enabling more efficient and targeted data retrieval. To distinguish between different disease classes from a multitude of data points, local maxima and NNS may be implemented to effectively identify distinct patterns and clusters within the dataset. Local maxima may be utilized as a signaling function in place of traditional classification methods. Local maxima focuses on identifying peaks within the data, which typically correspond to specific disease traits or classes. Using the detection of the local maxima, areas where data clustered can be found, reflecting distinct disease classifications. NNS is a robust mechanism for classification based on the premise that data points with similar attributes tend to cluster together, and a disease class can be inferred with a high degree of accuracy. Accuracy, precision, recall and the F1-score can then be used to assess the performance of the model.

Advantageously, the inventors have found that the combination of PCA with the use of a convolutional autoencoder provides detailed information regarding the cell aggregate structures, and enables an understanding of cell aggregate shapes in cell imaging data and their relationship to disease. These techniques provide a comprehensive approach, leveraging both the density distribution and inherent similarities among the data, enabling precise and reliable disease classification.

### Examples

The invention will be further clarified by the following examples, which are intended to be purely exemplary of the invention and are in no way limiting.

### Example 1: Convolutional Neural network

In this example, a CNN is used to classify images of single aggregates into one of three classes: ‘DLB’, ‘PD’ or ‘Healthy’. The ‘Healthy’ class may also be referred to as a ‘Control’ class (for example, when training the CNN).

It will be appreciated that a CNN includes convolutional layers. A convolutional layer moves a sliding window across the input image, aggregating the information from each section of the image, and repeats this process for the entire image to extract a set of features from the raw pixels, known as an activation map. This process enables dimensionality reduction of high dimensional image data whilst preserving information about the original image. Convolutional layers enable CNNs to derive features directly from images, rather than requiring a specialist to manually define a constrained number of image texture or shape features. The feature-agnostic nature of CNNs enables image classification and detection based on patterns of complex sets of features.

In this example, the CNN receives, in an input layer, an input corresponding to an image of a protein aggregate, and outputs an indication of whether the protein aggregate is associated with a proteopathy (in this example, the CNN outputs an indication that the protein aggregate is associated with a particular type of proteopathy, but this need not necessarily be the case). The input corresponds to a processed image of a protein aggregate. Types of pre-processing that may be used have been described above.

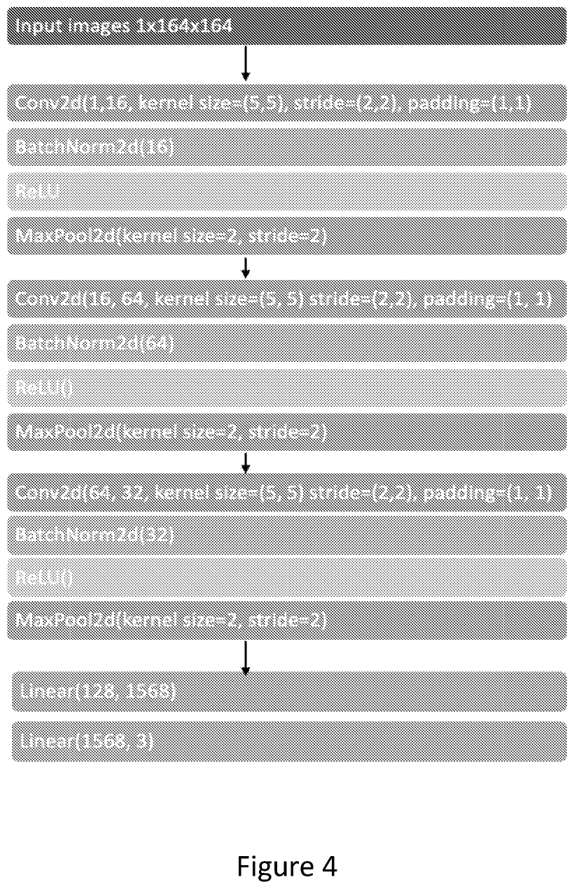

The architecture of the CNN used in the present example is illustrated in FIG. 4. However, it will be appreciated that any other suitable architecture for a neural network could alternatively be used. The CNN in this example has three convolutional modules, each comprising a convolutional layer, followed by batch normalisation, a Rectified linear unit (ReLu) activation function, and a max pooling layer. A 5×5 kernel is used, with a stride of 2 and a padding of 1, due to empty borders that may be present in the images. These layers may be followed by two dense layers.

The CNN architecture has several advantages. Batch normalisation standardises inputs to a layer in the network which stabilises the learning process, reduces the number of epochs required to train over, and provides some regularisation, therefore improving network generalisability. The max-pooling layers also provides regularisation by summarising features present in a region of the feature map, and reduces computational intensity. Moreover, in contrast to some other methods of classification, a CNN can use all of the information available in the image to perform the classification.

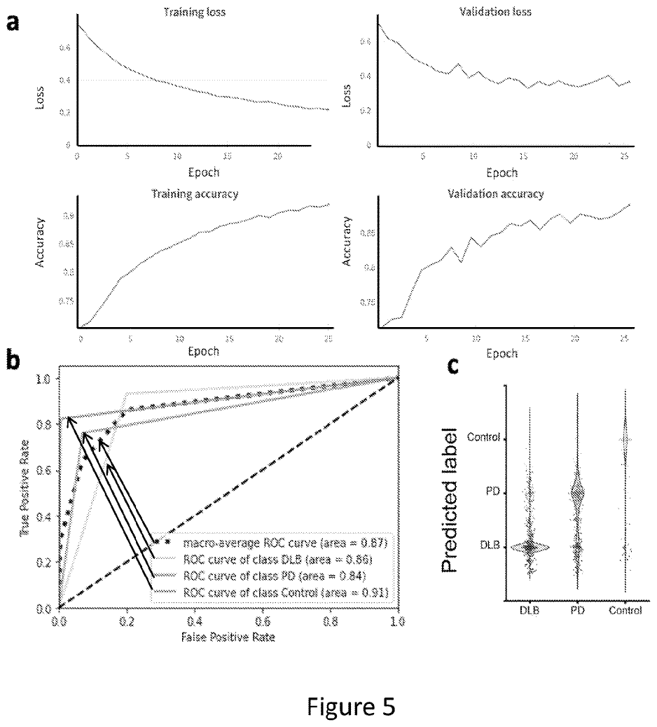

The CNN may be trained using a cross entropy loss function. To prevent over-fitting of the model, early stopping can be implemented when training the network. Training can be interrupted if the model shows no reduction in validation loss after ten epochs. The first five epochs of training may be ignored as a patience period, to account for the random initialisation of weights and biases in training. Training and validation loss, and training and validation accuracy, for a CNN is illustrated in FIG. 5A, which shows CNN training and validation accuracy and loss curves. FIG. 5B shows the receiver operating characteristic (ROC) curve for the model, and by area under the curve (AUC), controls show best performance, followed by DLB and PD. In this example, the CNN achieved 88.24% accuracy on an unseen test set of classifying 3403 images of protein aggregates. Table 1 shows the precision, recall, F1 score (harmonic mean of precision and recall), and number of images for each class.

TABLE 1
CNN performance

| Class | Precision | Recall | F1 | Samples |
|---|---|---|---|---|
| DLB | 0.91 | 0.94 | 0.93 | 2430 |
| PD | 0.78 | 0.72 | 0.75 | 747 |
| Control | 0.86 | 0.84 | 0.85 | 226 |

The high F1 scores and AUC values show that it is possible to distinguish between different synucleinopathies, and controls. The high F1 and AUC for controls indicates that control aggregates are visually distinct from diseased aggregates. High discriminative ability from single aggregate images indicates that they are good candidate biomarkers for classifying types of dementia.

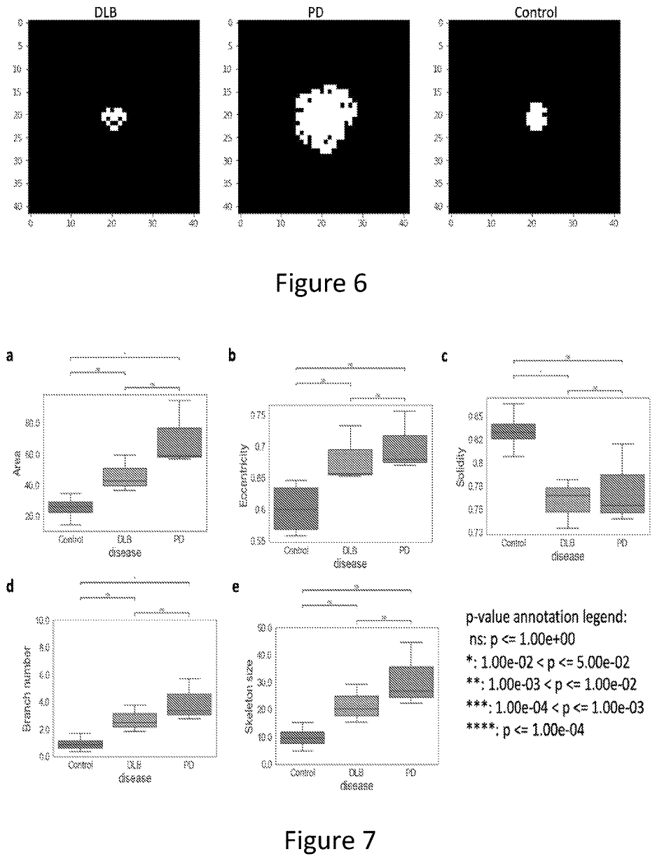

A corresponding violin plot is shown in FIG. 5C. In this Figure, the spread within each class is informative; the direction from the centre of the distribution is not informative, and the class plots are independent. The CNN performance indicates that diseases can be classified with high accuracy from single aggregates. Examples of aggregates from each class that were correctly classified with high confidence are shown in FIG. 6.

Attribution-based interpretation algorithms can be used to determine characteristics of aggregates in each class that drive the classification decisions. These methods include, for example: occlusion, gradientSHAP, Gradcam, and integrated gradients. Integrated gradients, occlusion, and gradientSHAP are single attribution algorithms, meaning they evaluate the contribution of each input feature to the model output, whilst GradCAM is a layer-based attribution approach. Occlusion is a perturbation based approach, where each contiguous rectangular region is replaced with a baseline value, and the difference in the network's output is computed for each. GradCAM is a layer based attribution approach which computes gradients for the target model output with respect to a chosen layer, then averages these by channel and multiplies these channel averages by the layer activations, before summing them. This result can be fed into a ReLU activation function so that no negative attributions are returned. Integrated gradients is a single attribution based approach, and computes the integral of gradients for a given input along the network path from baseline to output, again returning a heat-map of important features for the model's prediction. GradientSHAP computes expectations of gradients from random sampling of the distribution of the baselines, in order to approximate SHAP values. It is similar to an approximation of the integrated gradients method because it computes the expectations of gradients for different baseline values. In the present example, attribution-based interpretation algorithms confirm that it is the aggregate, and not a systematic difference in image background or pre-processing, that drives the classification.

To further improve the robustness of the model, the training and validation data sets may be augmented, for example, using random vertical and horizontal flips, and clockwise and anti-clockwise rotations, each with a probability of 0.5.

### Example 2: Non-Deep Learning Approach

As an alternative to using a CNN to identify aggregates associated with a proteopathy (or a particular type of proteopathy), a non-deep learning approach may alternatively be used. For example, an ensemble method that creates a series of decision trees using a random subset of features may be used. More generally, a classification method may receive morphological features of a protein aggregate as inputs, and output an indication of whether the protein aggregate is associated with a proteopathy.

Various morphological features can be identified from images of protein aggregates, and used in the classification method to classify the aggregates. These features include area, eccentricity, solidity, number of branches and skeleton length. Area is the total number of pixels inside the aggregate. In one embodiment, an input for the classification algorithm may comprise one or more fluorescence intensity values for pixels in the area of the image corresponding to the protein aggregate. Eccentricity is a measure of ellipticity: the ratio of the distance between focal points over the major axis when fitting an ellipse to the aggregate. Solidity is the proportion of pixels inside a convex hull fitted to an aggregate that are also within the aggregate. Skeleton size is the sum of the pixels in the skeletonized aggregate, where skeletonisation iteratively removes border pixels whilst maintaining connectivity. In other words, the skeleton size is the total number of pixels following skeletonization, where through successive passes, border pixels are removed until no more can be removed without breaking object connectivity. Skeletonisation is described above and, for example, in Tongjie Y Zhang and Ching Y. Suen—“A fast parallel algorithm for thinning digital patterns”—Communications of the ACM. The number of branches is the number of branching points in the skeletonized image, where a branch is a pixel connected to three or four other pixels. It will be appreciated that the morphological features of each protein aggregate may be extracted using any suitable image processing method (e.g. using any suitable image recognition software), such as OpenCV version 4.5.5 and/or Scikit-image version 0.19.2. It will also be appreciated that any suitable combination of the above-described morphological features may be used as the inputs for the classification algorithm. Moreover, any other suitable morphological feature(s) that can be obtained based on the imaging of the protein aggregates could alternatively, or additionally, be used. More generally, any suitable aggregate feature defining parameter could be used as an input for the classification algorithm.

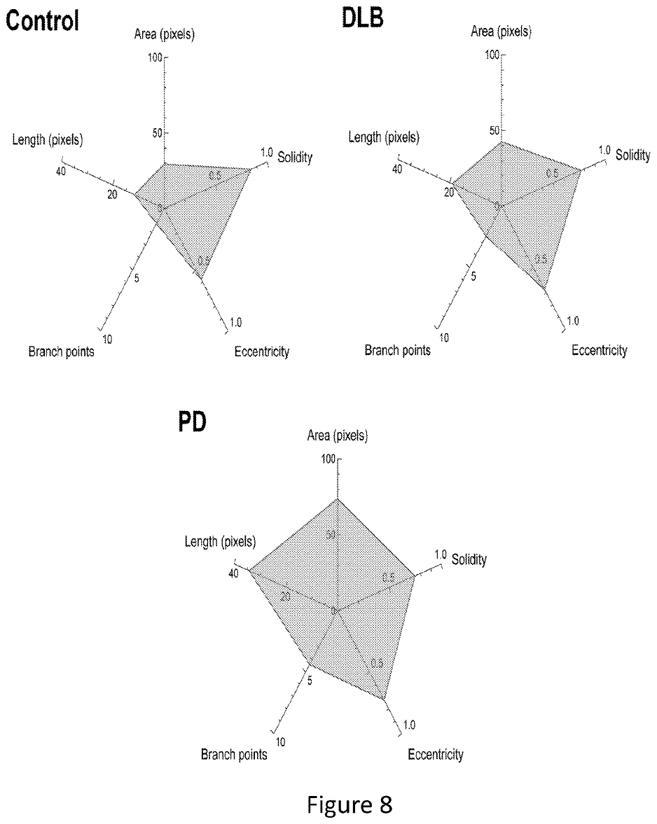

A distribution of the area, eccentricity, solidity, number of branches, and skeleton size for the aggregates of each disease is shown in box plots in FIG. 7. An average measure of each feature for each patient was used to account for nonindependence of aggregates from a patient. FIG. 7 shows the results of independent sample t-tests for significance of differences between classes with a bonferroni correction for multiple testing (because there are 3 classes). FIG. 8 shows the average values for each feature for control, Parkinson's Disease, and dementia with Lewy bodies samples. The inventors have realised that classification methods can be used to classify a sample as being associated with a proteopathy (or a particular type of proteopathy) based on the morphological features of single protein aggregates. However, the classification method is not limited to using a single protein aggregate, and the classification may be based on the classifications of a plurality of protein aggregates.

In this example, the classification method is a Random forest (or ‘random decision forest’) method, described for example in “Random forests”—Machine learning, 45(1):5-32, 2001. Hyper-parameters for the random forest can be tuned using grid-search 5-fold cross validation. A range of values tested for each hyper-parameter is shown in Table 2 below, along with the hyper-parameter definition. Grid search enables an exhaustive search over specified ranges of each hyperparameter, to find the combination of hyperparameters that generates the best model performance. 5-fold cross validation mitigates against over-fitting to the test set during grid-search, by splitting the dataset into five subsets and performing five different train test splits, with a different fold held out each time as the test set. In this example, the hyper-parameters of the model were entropy criterion; minimum two samples per leaf; minimum four samples per split; and one hundred estimators, tuned with cross validation. The value ranges for five-fold cross validation are shown in Table 2.

TABLE 2
Random forest hyper-parameter definitions and values tested

| Hyperparameter | Values tested |
|---|---|
| Minimum samples to be in a leaf | 2, 4, 6, 8, 10 |
| (external) node (‘min samples leaf’) | |
| Minimum samples to split an internal | 4, 6, 8, 10, 20, 50, 100 |
| node (‘min samples split’) | |
| Number of trees (‘n estimators') | 50, 100, 200, 400, 600 |
| Criterion to measure quality of split | Gini, entropy |

One or more (e.g. one or more, two or more, three or more, four or more, or five) of the area, eccentricity, solidity, number of branches and skeleton length may be input into the random forest model. For example, 4 or 5 morphological parameters from among the area, eccentricity, solidity, number of branches and skeleton length may be input into the random forest model. The model then outputs an indication of whether the protein aggregate is associated with a proteopathy (or a particular type of proteopathy).

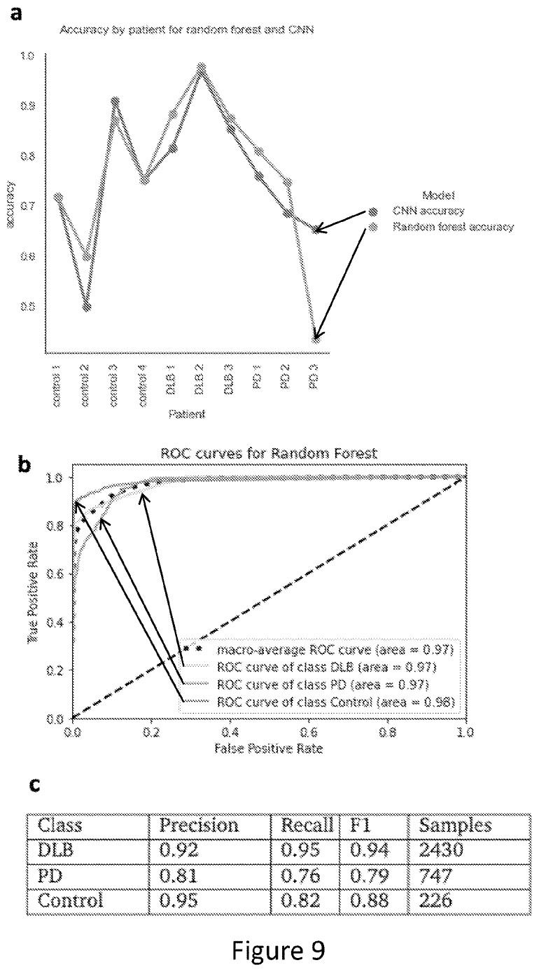

In the present example, the random forest method achieved an accuracy of 90% in classifying the images of the protein aggregates as corresponding to ‘DLB’, ‘PD’ or ‘Healthy’ samples, and an AUC of 0.97. Accuracy by patient for the random forest method and the CNN, the ROC curve for the random forest, and the random forest performance metrics are shown in FIG. 9. The CNN and the random forest model show a very high degree of correspondence in classification decisions. The random forest and CNN models made the same prediction on 93% of images in a test set, and 98% of their unanimous predictions were correct.

The output of the random forest method can be assessed using permutation-based feature importance. Permutation feature importance is the decrease in model score following random shuffling of the value of a single feature. The change in the model score indicates the extent of the model's dependence on that feature. Permutation-based feature importance results are shown in

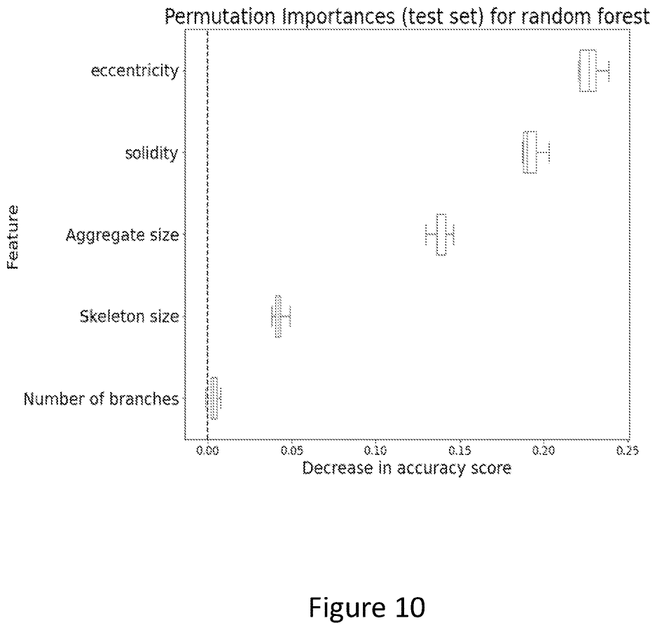

FIG. 10. FIG. 10 indicates that eccentricity, solidity, and area of the aggregate are particularly useful for performing the classifications.

### Cytotoxicity

Cytotoxicity of a sample may be measured using a lactate dehydrogenase (LDH) assay. In this example, aggregate samples or controls were added to 48-well plates each containing 30,000 HEK293A cells. 45 minutes before the end of the experiment, lysis buffer was added to the maximum LDH activity set of control cells. Following incubation at 37° C. for 4 or 24 hours, the media was centrifuged at 200×g for five minutes. 50 μl of the LDH assay buffer and 50 μl media were incubated for 1 hour, and 50|1 M acetic acid were used to quench the reactions. The absorbance at 490 nm and 680 nm were measured for each reaction on a plate reader.

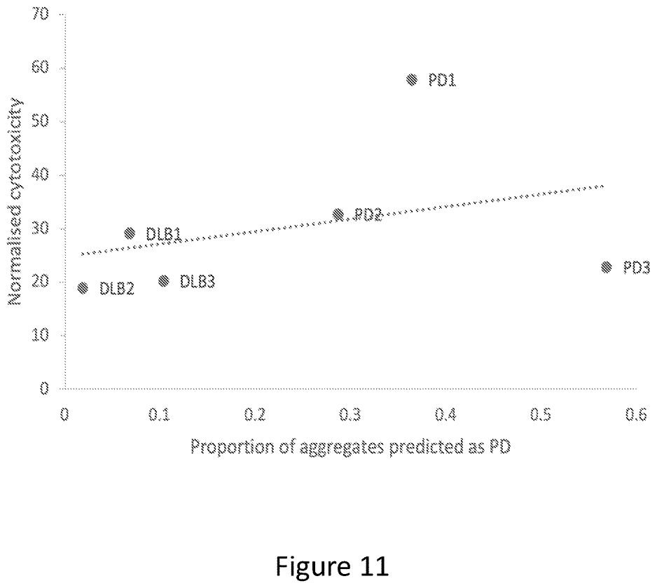

FIG. 11 shows the relationship between the proportion of an individual's aggregates classified as being associated with PD using a classification method (in this example, the CNN), and a normalised cytotoxicity score determined for that individual. FIG. 11 indicates that there is positive association between the proportion of PD-apparent aggregates and the cytotoxicity for an individual. FIG. 11 is consistent with the concept that the morphology of PD aggregates is relevant to their toxicity.

The inventors have realised that when one of the above-described classification methods is used to classify a plurality of protein aggregates, the relationship between the proportion of proteopathy-associated aggregates and the cytotoxicity can be used to determine and output a value of the cytotoxicity, based on the proportion of aggregates classified as corresponding to the proteopathy (in this example, PD).

### Example 3—Imaging of Aggregates

The inventors used fluorophores ProteoStat and Amytracker 630 (AT630) for quantitative SMLM imaging of aggregates at ~4 nm precision. AT630 staining enabled sensitive and quantitative detection of aggregate species down to ~10 nm in size in live cells and ex vivo brain tissue. The inventors demonstrate herein that aggregates found in live HEK293A cells or in fixed mouse brain sections can be super-resolved following AT630 staining. Critically, AT630-staining revealed that the plasma membrane effectively prevented aggregates over 450±60 nm from entering the cell. Aggregates that invaded the intracellular space were quantitatively characterized. The size of the membrane-penetrating aggregates detected by AT630 is shown to be correlated with cytotoxicity. The inventors validated this observation and quantified 450 nm aggregate toxicity using PD- and DLB-derived aggregates, showing that αS aggregates differ in potency and in toxicity depending on the pathology they originated from. The method described herein enables detection of different aggregate species through SMLM characterization and provides a straightforward and reliable in situ approach to quantify the fraction of toxic aggregates.

### Results and Discussion

Sensitive Aggregate Staining with ProteoStat and AT630

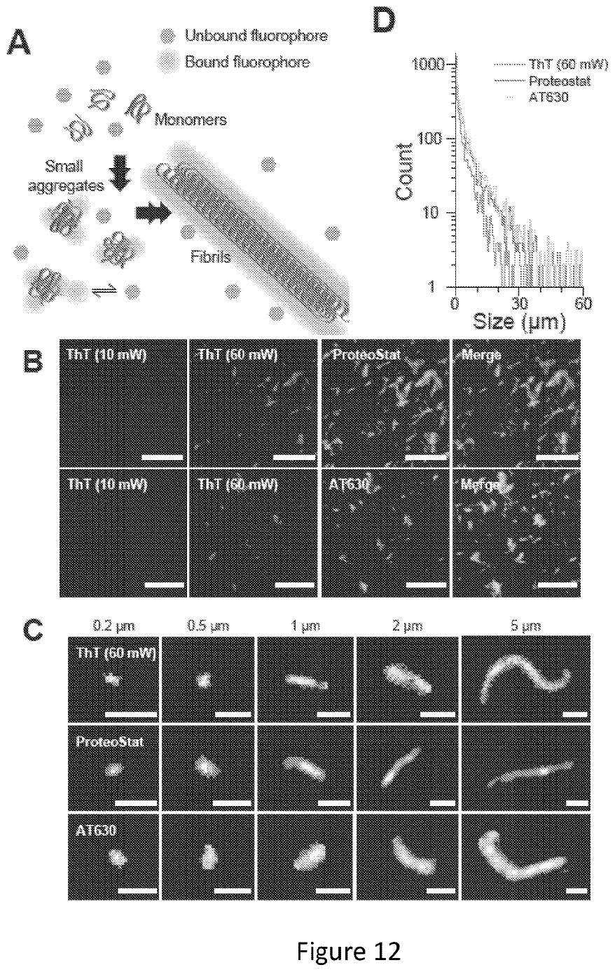

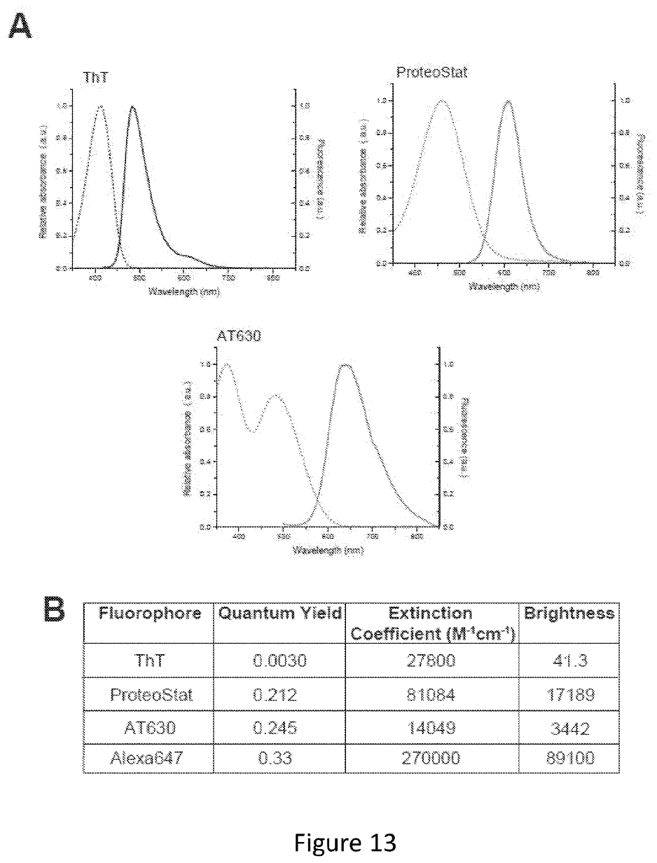

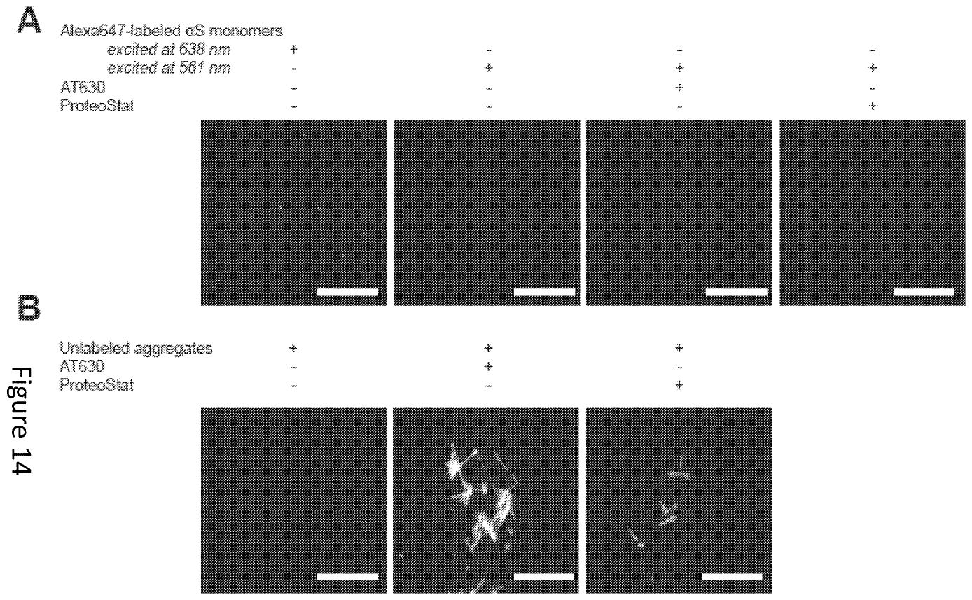

The specificity of ProteoStat and AT630 for different aggregate species was determined first and compared with the widely used Thioflavin T (ThT). Since the excitation wavelength used and the emission maximum of ThT differs from those of ProteoStat and AT630 (FIG. 13A), it was possible to co-stain aggregates assembled from recombinant αS with ThT and ProteoStat, or with ThT and AT630 (FIG. 12A). Assembled aggregates were imaged on a custom-built total-internal reflection fluorescence (TIRF) microscope, following protocol described in Cliffe, R. et al. Cell Rep 26, 2140-2149 e2143 (2019) (see Materials and Methods). The photophysical properties for all three fluorophores were determined and revealed that both ProteoStat and AT630 were brighter than ThT (FIG. 13B), which required a six-fold increase in excitation laser power to reach an acceptable level of detection (FIG. 12B). Merging the same field of view staining of ProteoStat and ThT or AT630 and ThT showed complete overlap of aggregates in the two images, indicating that ProteoStat and AT630 both detected all aggregate species recognized by ThT (FIG. 12B). As controls, the fluorescence emission in the absence of aggregates, in the presence of the aggregates but absence of fluorophores, or in the presence of monomeric αS and fluorophores, was shown to be negligible (FIG. 14). This indicates that both fluorophores have a high specificity for aggregate structures and high signal-to-noise ratios.

The imaging approach described herein detected a wide range of αS aggregate sizes by fluorophore staining (FIG. 12C). Aggregate size was previously defined from TIRF imaging as the longest distance that can be measured within an aggregate entity, defining species <1 μm as “small aggregates” and “fibrils” for species >1 μm (see Materials and Methods). Importantly, in addition to characterizing large fibril structures, small aggregates, whose molecular features reach below the ~200 nm diffraction-limit, were easily identified even at low laser power (10 mW unless stated otherwise). The abundance of aggregates detected with all three fluorophores is plotted by size (FIG. 12D). AT630 and ProteoStat were able to detect a significantly greater number of aggregates at the same aggregate concentration. The inventors concluded that both ProteoStat and AT630 are more adept at uniformly staining aggregates than ThT (FIG. 12D) and require less laser power for reliable aggregate detection, thus reducing potential phototoxic damage to biological samples.

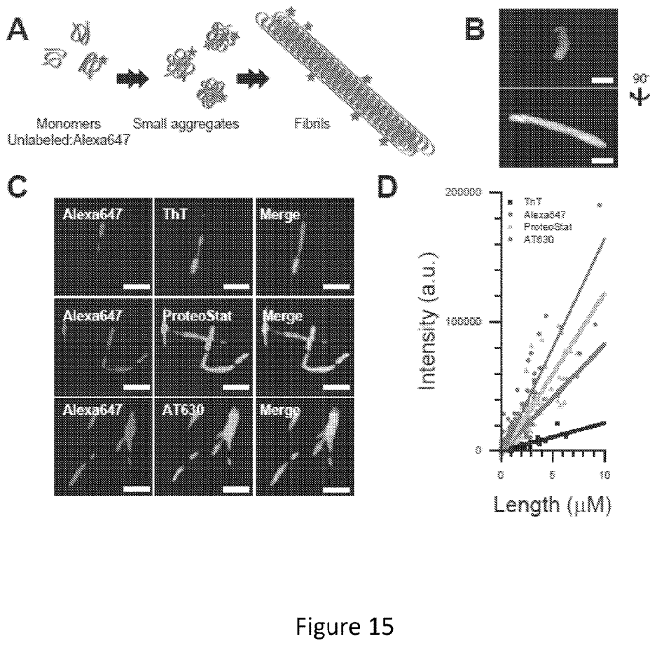

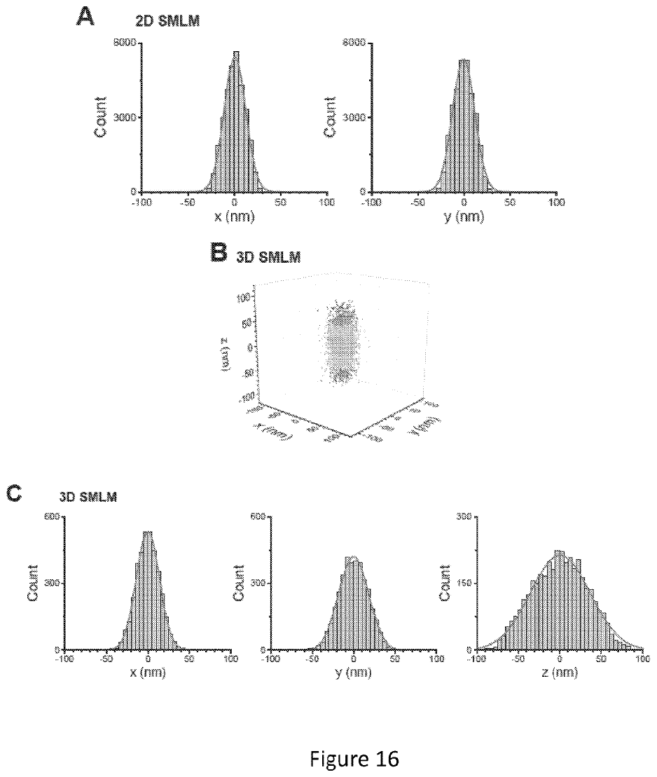

To account for the presence of internal structures and aggregate conformations that might not have been recognized by ThT in FIG. 12B, aggregates were assembled from αS monomers covalently labelled with Alexa647. A strategy of mixing different fractions of Alexa647-labeled and unlabeled αS was developed to overcome potential steric interference that Alexa647 may have on αS aggregation, which could prevent assembly of fibrils. This fraction (10% Alexa647-labeled αS) was optimised to allow formation of fibrils that incorporated Alexa647-labeled αS (FIG. 15A). Fibril structures were reconstructed in 3D using an astigmatism SMLM imaging method, which confirmed that labelling was largely uniform over the fibril structures (FIG. 15B). The SMLM approach described herein achieved 21±1 nm planar and 84±2 nm axial resolution (see Materials and Methods and FIG. 16). ProteoStat and AT630 both stained Alexa647-labeled αS aggregates, and the detected features matched with the fluorescence detected from Alexa647 (FIG. 15C). These results confirm that ProteoStat and AT630 detect all aggregate species present in the sample.

Imaging individual aggregates allowed the relationship between the total fluorescence intensity detected from each aggregate versus its size to be examined (FIG. 15D). The total fluorescence intensity detected from each aggregate can be assumed as a product of the fluorophore brightness and the number of fluorophores bound to each aggregate. Both ProteoStat and AT630 demonstrated greater fluorescence intensities with aggregate size compared to ThT and Alexa647-labeling in FIG. 15D. Since the brightness of ProteoStat and AT630 are greater than that of ThT but not as bright as Alexa647 (FIG. 13B), the increased total intensities of ProteoStat and AT630 would be expected to originate from higher densities of fluorophores bound to each aggregate. The increased density of fluorophores reflects an expected higher density of amyloid structures than the 10% of monomers that were labelled with Alexa647. Overall, this data suggests that AT630 and ProteoStat more sensitively detect amyloid structures than Alexa647 and ThT. Furthermore, this data highlights the advantages of aggregate-activated fluorophores over the Alexa647-conjugation approach.

Super-Resolution of Aggregates with ProteoStat and AT630 at ~4 nm Precision

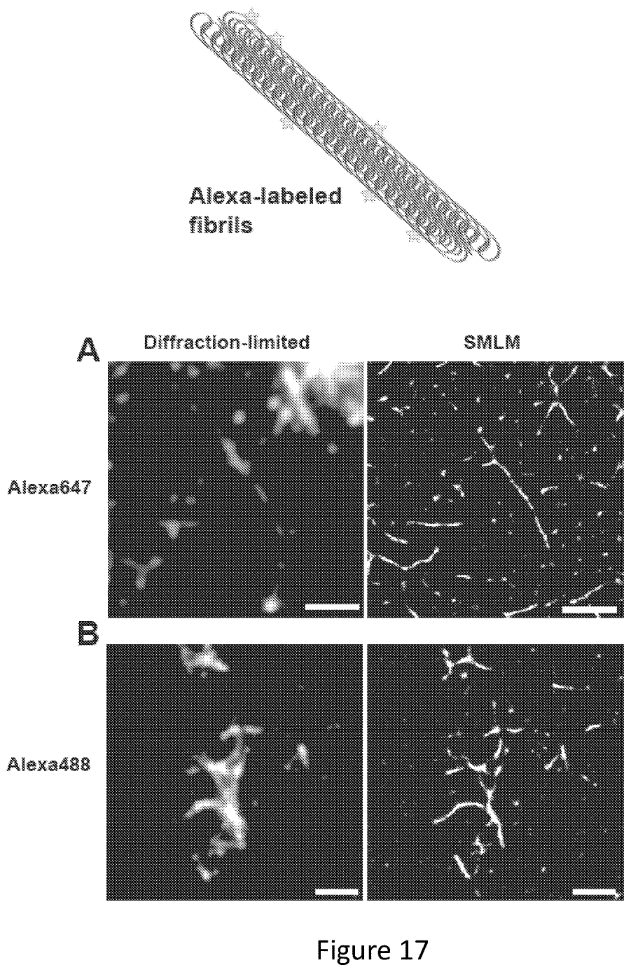

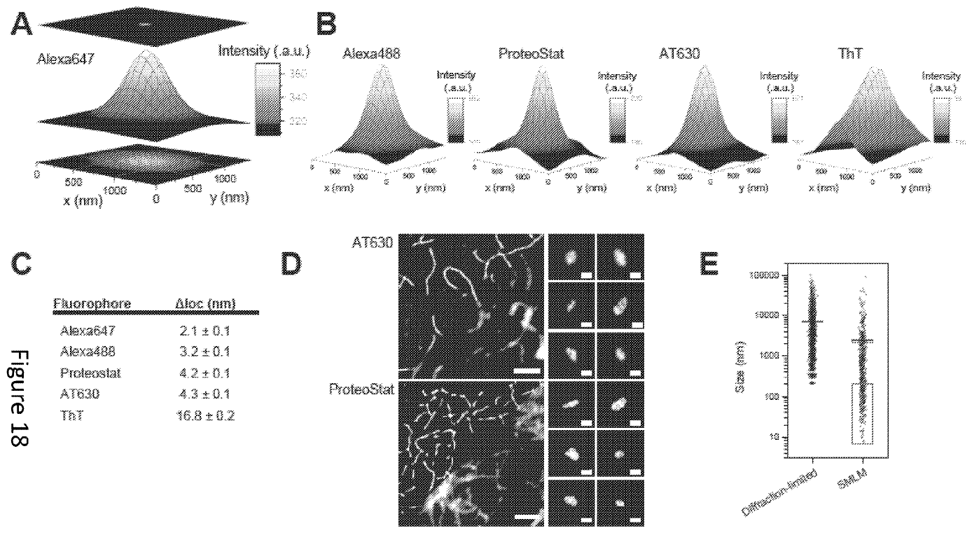

Accurately determining the size of aggregates below ~200 nm is challenging due to the Abbe diffraction limit. SMLM was therefore used to characterize the structural features of aggregates in finer detail. Initially, aggregates in 2D (FIG. 17) and in 3D (FIG. 15B) were imaged using fibrils labelled with Alexa647 or Alexa488. High laser-powers and an imaging buffer containing a reducing agent and an oxygen scavenger system (see Materials and Methods) were used to induce the fluorophores to stochastically switch on and off, allowing densely packed single-molecules to be distinguished temporally. The point-spread-functions (PSFs) measured from the emission of individual fluorophores were fitted to Gaussian profiles, and these were used to calculate the position of each fluorophore with greater accuracy. FIG. 18A shows the average PSF profile of Alexa647-conjugated on αS aggregates collected for SMLM. The localization precision (Δloc) was determined from the number of photons detected and the full width half maximum (FWHM) of the PSF profile of Alexa647 (see Materials and Methods). Similarly, average PSFs were used to calculate the localization precision of Alexa488, ProteoStat, AT630 and ThT (FIG. 18B). Alexa647 and Alexa488 achieved the highest localization precisions at 2.1±0.1 nm and 3.8±0.1 nm, respectively (FIG. 18C). The average PSF profiles of AT630 and ProteoStat also gave excellent signal-to-noise profiles and achieved localization precisions (4.2±0.1 nm and 4.3±0.1 nm, respectively) in range with the Alexa fluorophores. As expected, the localization precision determined for ThT was lower, achieving 16.8±0.2 nm.

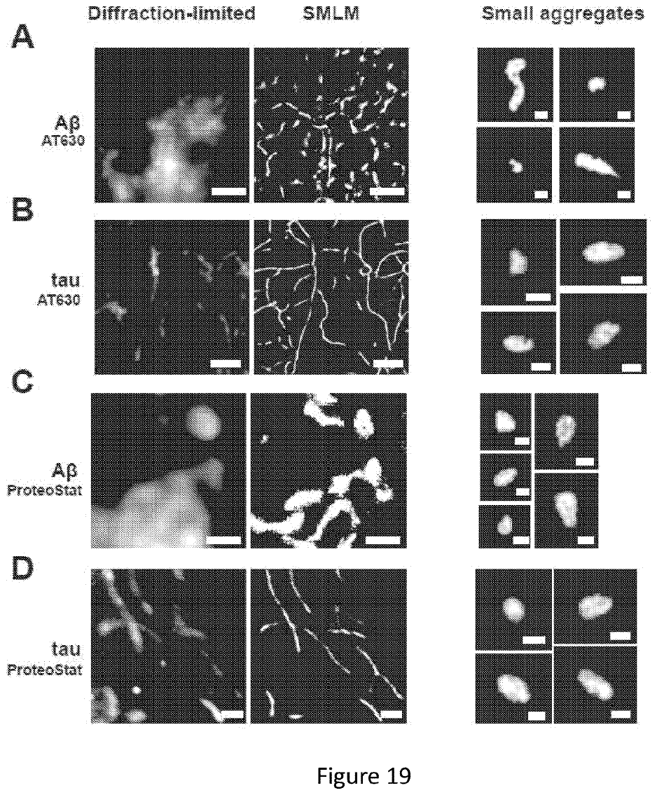

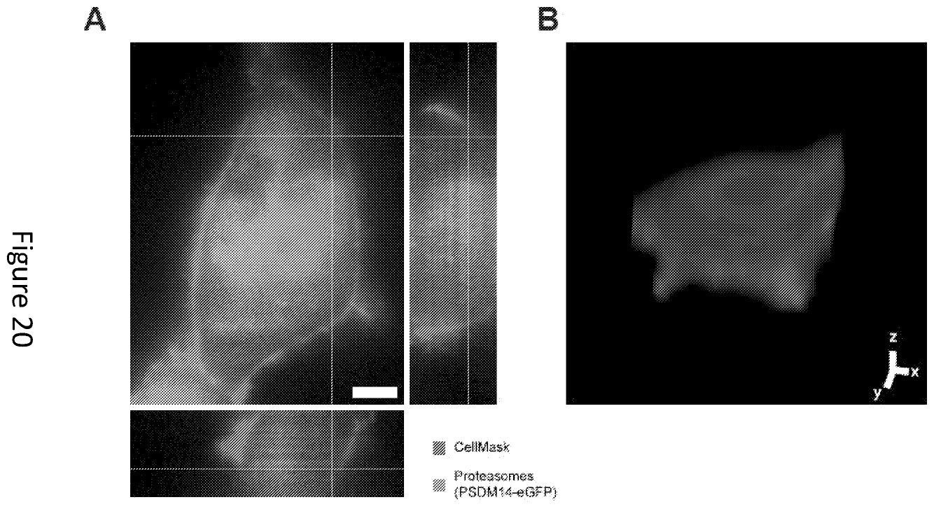

ProteoStat and AT630 both enabled SMLM imaging of αS aggregates assembled in vitro and revealed structural details of aggregates from >10 μm down to 10 nm in size (FIG. 18D-E). The size of each aggregate was defined by thinning the aggregate shape to a path one pixel in width, and calculating the aggregate size as the length of this one-pixel wide path (see Materials and Methods). From the reconstructed SMLM images, the smaller aggregates within a few hundred nanometers in size were found to be heterogeneous in structure, some appeared globular whilst others were elongated and with apparent differences in ellipticity (FIG. 18D). Similar observations were made using the same staining approach to super-resolve aggregates assembled from tau or AB, demonstrating that sensitivity of detection was not dependent on the misfolded protein involved (FIG. 19). These results together indicate that ProteoStat and AT630 enable SMLM imaging of molecular features of aggregates at ~4 nm localization precision, resolving the conformational heterogeneity in aggregate samples down to the same order of magnitude as the organic fluorophores.

### Aggregate Species Below 450±60 nm Readily Penetrate Plasma Membranes and Invade HEK293A Cells

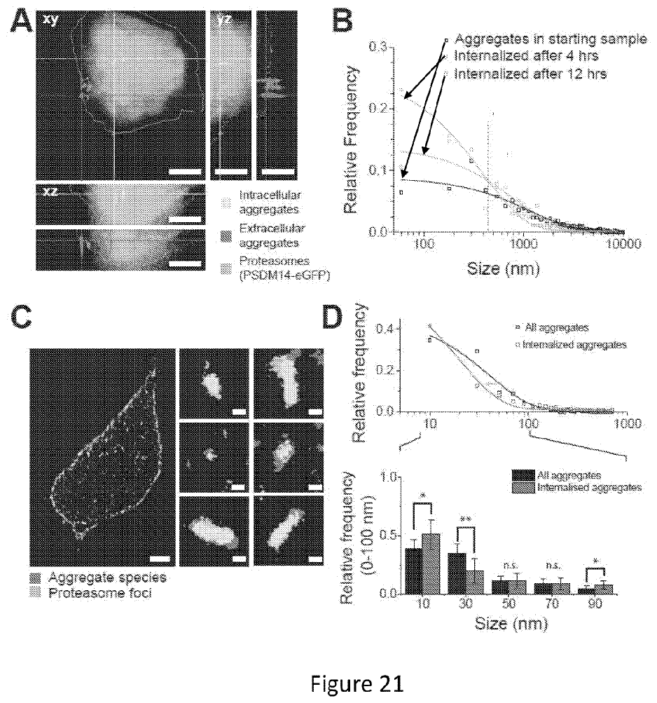

To test whether the new fluorophores detect aggregates within a cellular environment, recombinant aS aggregates were incubated with HEK293A cells. αS aggregates released into the extracellular domain are known to be taken up by cells. Here, a CRISPR-engineered line was used in which the genomic loci coding for a proteasomal subunit, PSMD14, was knocked in with an eGFP-coding sequence introduced to the 3′-end in frame with the gene, thereby fluorescently tagging endogenous proteasomes. AT630 was used to stain aggregates in live cells. Staining time and washing procedure were optimized for AT630 to achieve the highest signal-to-background ratio for imaging, and the TIRF microscope was adjusted to apply a highly inclined and laminated optical sheet (HILO) imaging approach, which enabled axial illumination in the z direction throughout the whole cell volume (see Materials and Methods). The boundaries of the cell were defined from z-stacks of images taken 100 nm apart throughout the cell volume. Since proteasomes are dispersed throughout the cell interior, a rolling ball filter was applied to the eGFP emission to reconstruct the cell volume, and this approach was validated by comparing cell volumes detected from eGFP emission and a CellMask™ Plasma Membrane Stain (FIG. 20). From the eGFP emission, aggregates that had entered the cell could be distinguished from those that lay on the apparent apical side of plasma membrane (FIG. 21A). After 4 hrs incubation, mostly smaller aggregates sub-micrometer in size (FIG. 21A) were detected within the intracellular domain, while the larger fibrils remain docked to the plasma membrane (FIG. 21A). The relative frequency of internalized species was plotted by size, showing that the distribution of these aggregates was best described by an exponential function (FIG. 21B). This distribution was compared to the internalized species after 12 hrs, in which a greater frequency of larger aggregate species entering the cell was observed (FIG. 21B). As a comparison, the function based on the total number of aggregates detected in the starting sample was plotted, i.e. sample added at 0 hrs (black curve, FIG. 21B). The starting sample showed a lower relative prevalence of smaller aggregates than in the functions for both 4 and 12 hrs, suggesting a higher efficiency of these smaller aggregate species at crossing the plasma membrane. Given that the relative frequency of internalized aggregates at 4 and 12 hrs show different distributions, the intersection at 450±60 nm between the two curves represents the aggregate species that does not change in relative abundance over time (dashed line, FIG. 21B). Following this reasoning, the crossover was interpreted as a threshold to define a population of aggregates with sizes smaller than 450±60 nm (aggregate₄₅₀ₙₘ) that is able to traverse the cell membrane with increased efficiency.

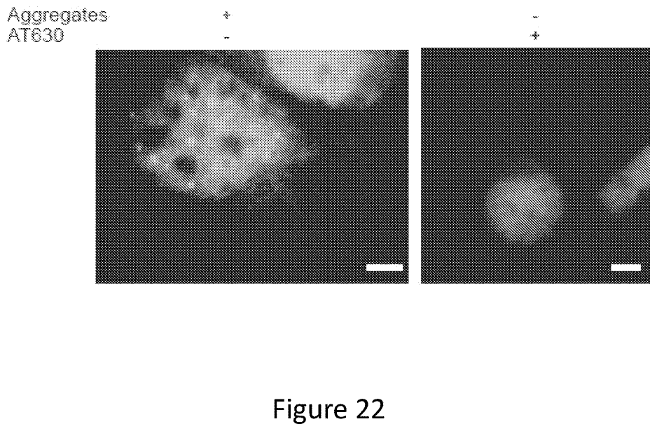

Sonication has been shown to break apart fibrils and increase the abundance of smaller aggregates in the mixture, therefore the aggregate invasion experiment was repeated with sonicated fibrils and the fate of aggregate₄₅₀ₙₘ was followed after incubation for 24 hrs. The dispersed proteasomes were observed to assemble into foci structures (FIG. 21C), and areas of increased proteasome concentration were observed to condense around internalized aggregates. Staining these aggregate-treated cells with AT630 followed by SMLM imaging (in Fluorobrite rather than SMLM imaging buffer, which is toxic to cells) revealed that many proteasome foci indeed colocalized with internalized aggregates. The AT630 staining performed in live cells was specific, as no meaningful fluorescence was detected in the absence of aggregates (FIG. 22).

The size of internalized oligomers was determined by SMLM and the size distribution was compared with the aggregates before they were deposited on the cells (FIG. 21D). A left shift in the distribution of intracellular aggregate size by 21±3 nm was detected (arrow showing shift from black to grey curve, FIG. 21D), indicating that internalized aggregates became smaller than before they were deposited on the cells. Focusing on aggregates with sizes <90 nm (inset, FIG. 21D) the inventors found by using a two-sample t-test that, unlike the larger size groups, the smallest size group (10 nm) showed a marked increase in their relative frequency once internalized. This is in line with the left shift of the curve in FIG. 21D and suggestive of gradual disassembly/degradation of larger aggregates into the smaller species once internalized. Additional mechanisms may be involved in removing the smallest size group. Together, the data indicate that proteasomes target internalized aggregates, and the reduction in aggregate size in cells after 24 hrs suggest the observed foci contain active proteasomes. These results provide evidence for aggregates to promptly attract proteasomes, thus increasing the local degradation activity to facilitate aggregate removal.

Given that aggregates inside cells are typically observed after 4 hrs, the data suggest that the plasma membrane is readily permeable to smaller aggregates and filters out aggregates and fibrils above 450 +60 nm in size. Given the uncertainty range, it is likely that in reality this threshold represents a critical range of aggregate sizes, and aggregates that are significantly larger than this range are less likely to be internalized. The plasma membrane therefore serves as an effective protectant against larger fibrils (>450±60 nm) whilst remaining vulnerable to small aggregates. Without wishing to be bound by theory, the inventors hypothesis that the reverse effect is also true—the plasma membrane may effectively contain the larger fibrils while small aggregates escape into the extracellular domain, if below 450±60 nm, and spread to adjacent cells.

αS Aggregates Below 450±60 nm Correlate with Cytotoxicity

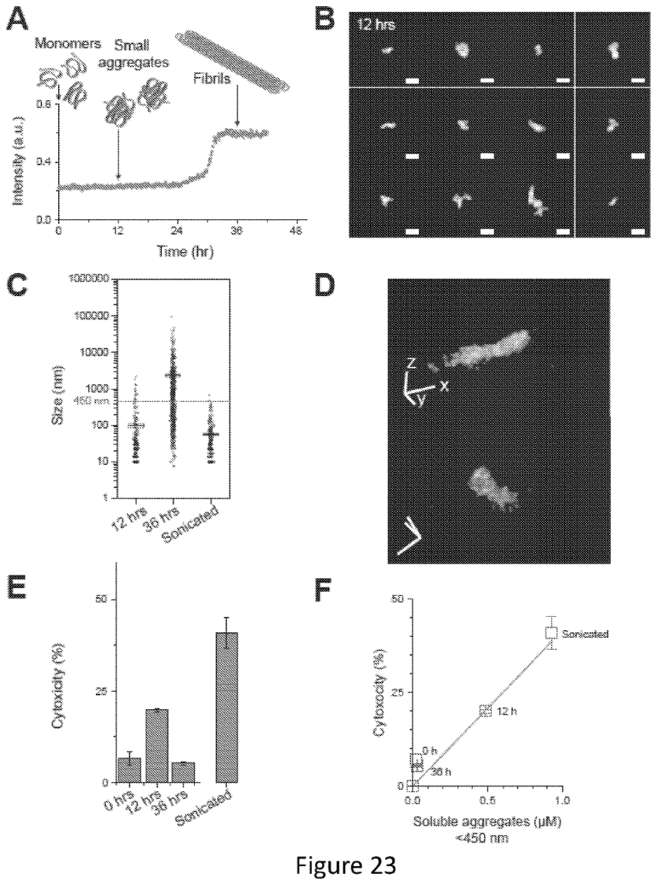

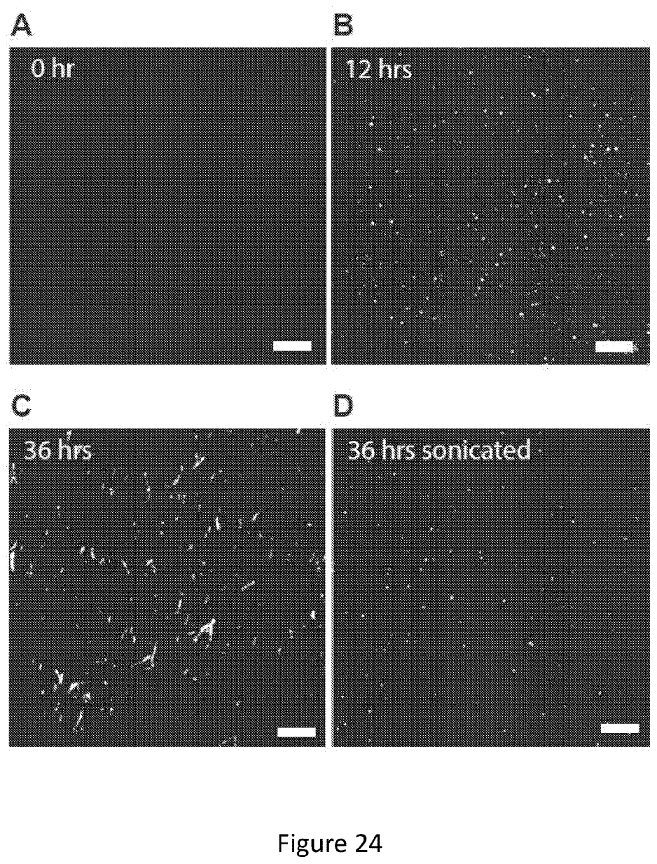

To determine whether the invasive aggregate₄₅₀ₙₘ identified in FIG. 21 may cause cytotoxicity, a controlled αS aggregation reaction was set up in vitro. The aggregation process of recombinant αS has been characterized previously (Chen, S. W. et al. PNAS 112, E1994-E2003 (2015); Ye, Y., Klenerman, D. & Finley, D. Journal of Molecular Biology 432, 585-596 (2020); and Iljina, M. et al. Sci Rep 6, 33928 (2016)), and these studies were used to guide the selection of timepoints at which aliquots should be taken (FIG. 23A). No aggregates were expected at time 0 hrs, when only monomers should be present, and mostly small aggregates were expected at 12 hrs after aggregation was initiated, and before any significant increase in ThT signal was observed. A larger proportion of fibrils was expected at 36 hrs after the aggregation reaction had entered the stationary stage. Indeed, a predominant population of smaller aggregate species was detected after 12 hrs using AT630, no aggregates were observed at 0 hrs, and large numbers of fibrils were found at 36 hrs (FIG. 23B-C and FIG. 24A-C). Again, a sample of fibrils from the 36 hrs timepoint was sonicated to generate a large population of smaller aggregates similar in size to those formed after 12 hrs (FIG. 24D). The detected smaller aggregate species had distinctive structures, which were super-resolved in 2D and 3D (FIG. 23D). Next, HEK293A cells were treated with a calculated equivalent αS monomer concentration of 1 μM taken at the different timepoints, and cytotoxicity was measured using a lactate dehydrogenase (LDH) assay (FIG. 23E), which offers sensitive readouts of membrane permeabilization and LDH release. The aggregate sample at 12 hrs time point was most harmful to cells, with 20.2±0.6% cytotoxicity value of the positive control for the assay (lysis buffer treatment of cells). The monomer- and fibril-rich samples at 0 hrs and 36 hrs, respectively, induced smaller cytotoxic responses (5.5±0.7% and 7±2%, respectively) in comparison. As expected, sonicated fibrils from the 36 hrs timepoint resulted in significant cell death (41±5% cytotoxicity, FIG. 23E). These results demonstrate that the size of the aggregates plays a prominent role when inducing cytotoxicity.

The inventors subsequently sought to quantify the concentration of αS proteins that constitute the aggregate₄₅₀ₙₘ population at each timepoint. SMLM was used to determine aggregate size and the fraction of aggregate₄₅₀ₙₘ was calculated from the total size of all aggregates in the sample compared to the sum of total sizes detected from aggregate₄₅₀ₙₘ. Plotting the calculated concentrations of αS in aggregate₄₅₀ₙₘ versus the measured cytotoxicity of the different samples allowed a linear relationship to be determined (FIG. 23F), suggesting that the concentration of aggregate₄₅₀ₙₘ causes proportional cell damage within this range of concentrations. These data therefore imply that aggregate₄₅₀ₙₘ contains the most toxic species.

AT630 Stains Pathological Aggregates Derived from Parkinson's Disease and Dementia with Lewy Bodies

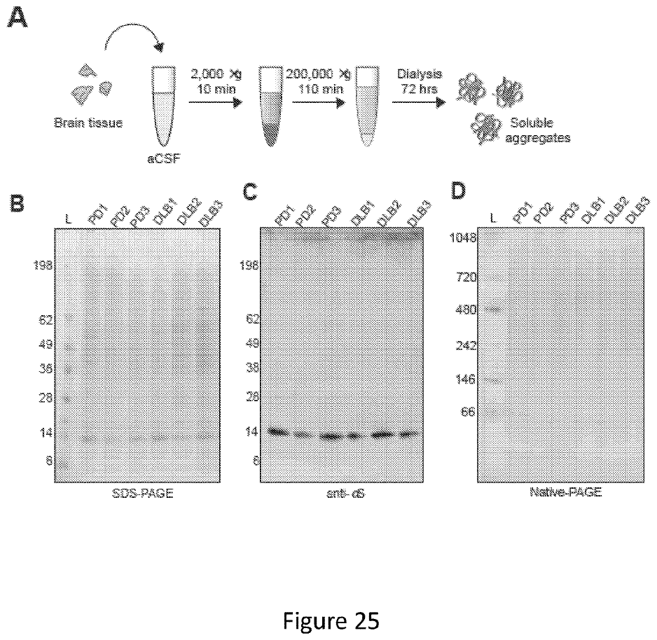

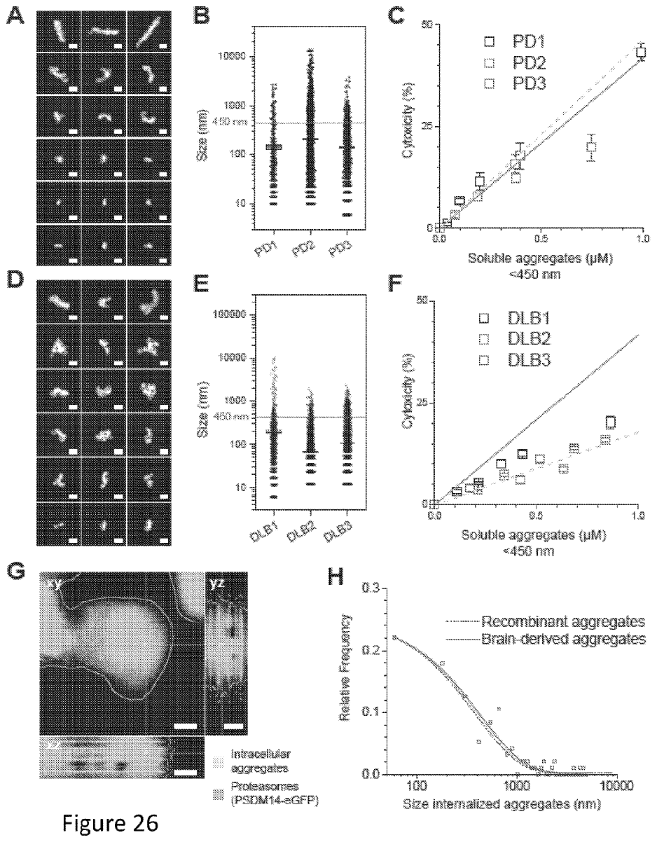

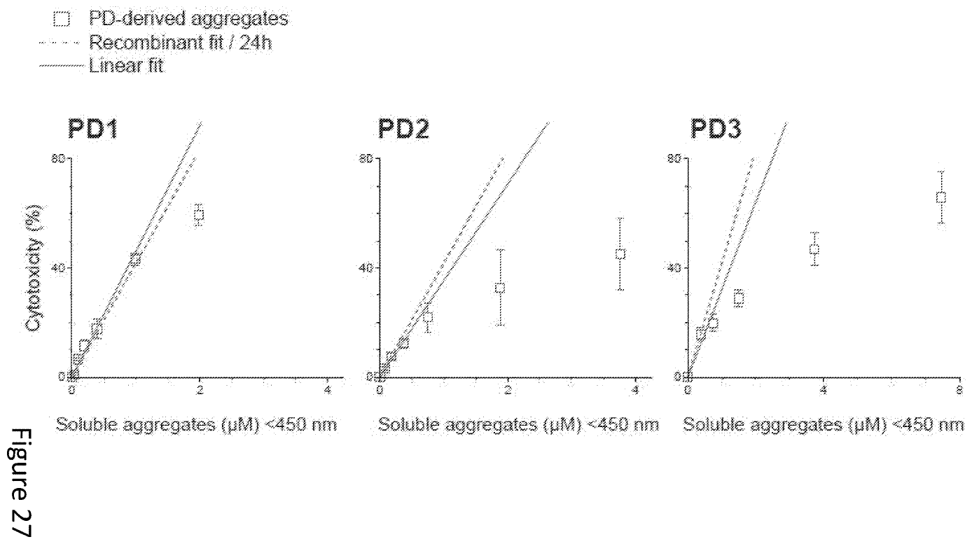

To assess the cytotoxicity-aggregate₄₅₀ₙₘ correlation established in FIG. 23F, brain soaks were performed on post-mortem cortical brain tissues from three PD donors by modifying an existing protocol (Hong, W. et al. Acta Neuropathologica 136, 19-40 (2018)) (FIG. 25). Following this optimized protocol, AT630 efficiently stained the extracted aggregates and enabled imaging by SMLM. Detailed structural features of typical tissue-derived aggregate 450 nm are shown in FIG. 26A. All three PD samples contained predominantly smaller aggregate species with mean sizes±standard error of mean (SEM) of 140±10 nm, 211±7 nm and 106±2 nm for PD1, PD2 and PD3 respectively (FIG. 26B). SMLM was used to calculate the fraction of aggregate₄₅₀ₙₘ in all three samples (11%, 12% and 20% respectively) by applying similar calculations as in FIG. 23, from which the estimated aggregate₄₅₀ₙₘ concentration was determined. Cytotoxicity was next measured for increasing concentrations of each sample and plotted against the aggregate₄₅₀ₙₘ concentration (FIG. 27). At higher sample concentrations of brain-derived aggregate₄₅₀ₙₘ, the amount of aggregates larger than 450 nm also increases and may interfere with the potency of aggregates₄₅₀ₙₘ, potentially perturbing the linear relationship with LDH cytotoxicity. However, within the linear sections of each cytotoxicity curve, slopes were observed that were largely in agreement with the cytotoxicity-aggregate₄₅₀ₙₘ relationship in FIG. 23F with gradients of 42±6 μM-1 and 46±4 μM-1 for recombinant αS and PD samples, respectively (FIG. 26C and FIG. 27).

The inventors subsequently investigated if aggregate₄₅₀ₙₘ species derived from post-mortem brain tissues of three donors with DLB also obeyed the same correlation with size and cytotoxicity (FIG. 26D-F). Examining the size distribution, the mean±SEM aggregate sizes for DLB samples were 200±20 nm, 66±2 nm and 109±2 nm, respectively, and smaller than those observed for PD, and with high fractions of aggregate₄₅₀ₙₘ within each mixture (43%, 84% and 69%, respectively). While the corresponding cytotoxicity values do not fall on the same linear slope shared by the recombinant as and PD samples, they remain linearly correlated with aggregate₄₅₀ₙₘ concentration with a gradient of 17.8±0.6 M-1 (FIG. 26F).

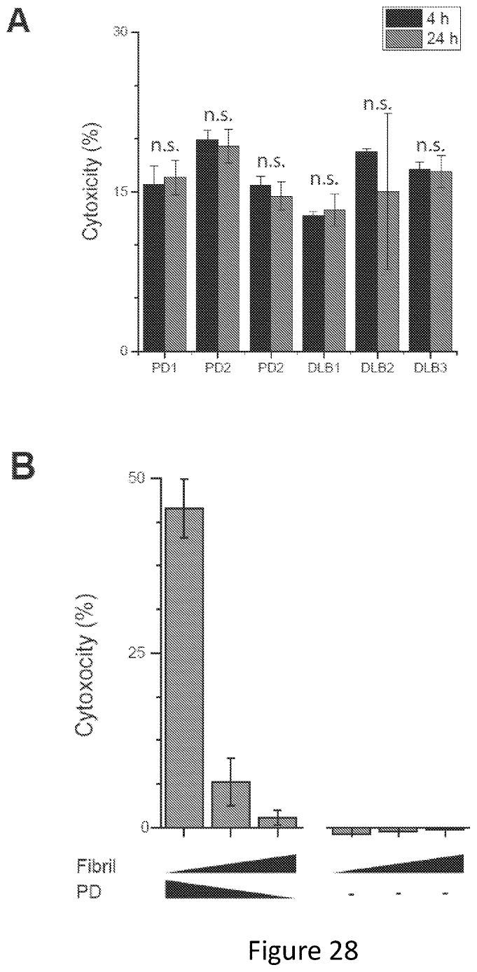

The inventors further compared the cytotoxicity of 1 μM PD and DLB samples after 4 and 24 hrs (FIG. 28A), and found no significant difference between the two incubation periods, supporting the view that the aggregates that penetrate cells at 4 hrs are responsible for the observed toxicity. The cytotoxicity level did not depend on the effective concentration of fibrils larger than 450±60 nm but on small aggregates (FIG. 28B), suggesting that any toxic effects resulting from larger fibrils in the brain may involve more complex pathways in vivo. This supports the conclusion that the size, rather than concentration of total aggregates, determines their innate ability to harm cells. To validate the toxicity of small aggregates, 1 μM PD patient-derived aggregates (PD1) were incubated with HEK293A cells expressing PSMD14-eGFP for 4 hrs. The distribution of PD1 aggregate sizes inside cells was similar to that of intracellular recombinant S aggregates as found in FIG. 21 (FIGS. 26G and H). This supports the observed similarity in toxicity gradient of recombinant and PD aggregates in FIG. 26C, again confirming that the small aggregate species exert toxicity by penetrating the plasma membrane.

Together, the cytotoxicity data demonstrate that the αS aggregates present in different synucleinopathies may possess distinct potency in causing cell damage. This could be important to explain why different pathologies arise from misfolding and aggregation of the same protein. The results described herein validate the robustness of using the SMLM approach to determine cytotoxicity based on the fraction of aggregate₄₅₀ₙₘ within a heterogeneous aggregate sample. The SMLM approach may further be applied to other biological samples and fluids, through which toxicity may be determined in a similar manner.

### AT630 Stains Aggregate Species in Mouse Brain Tissues

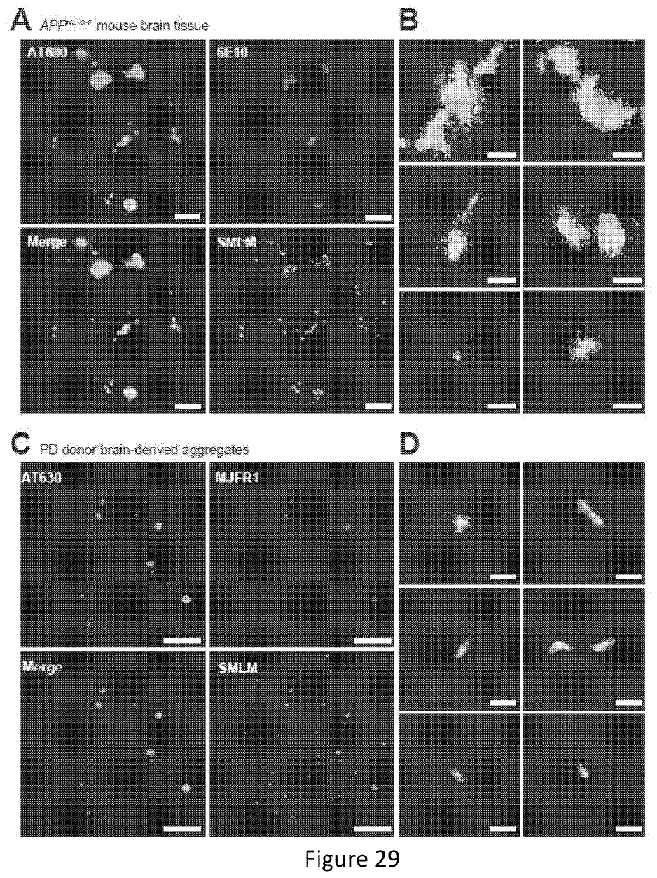

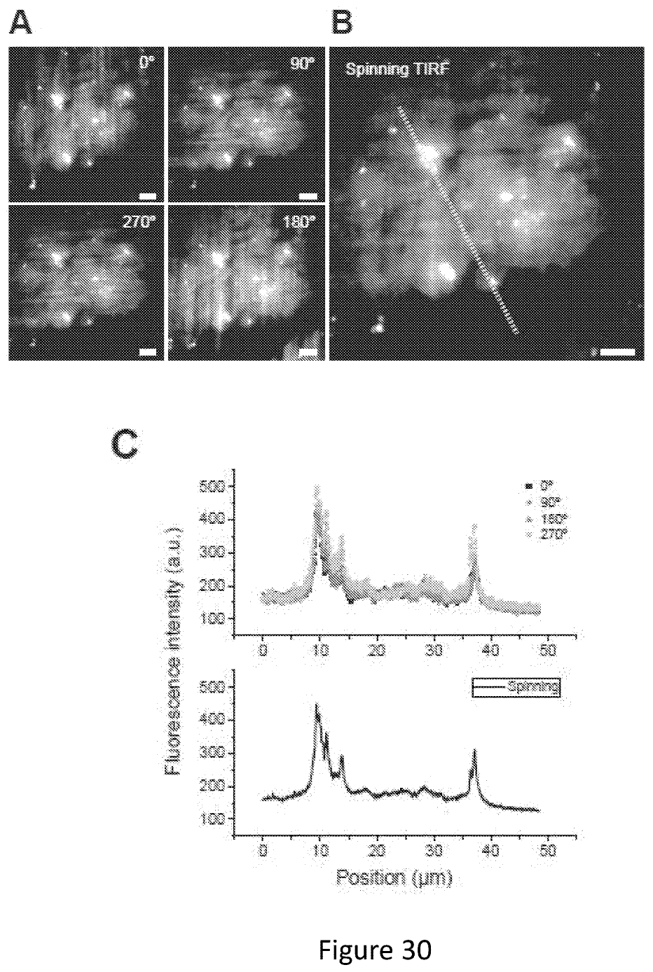

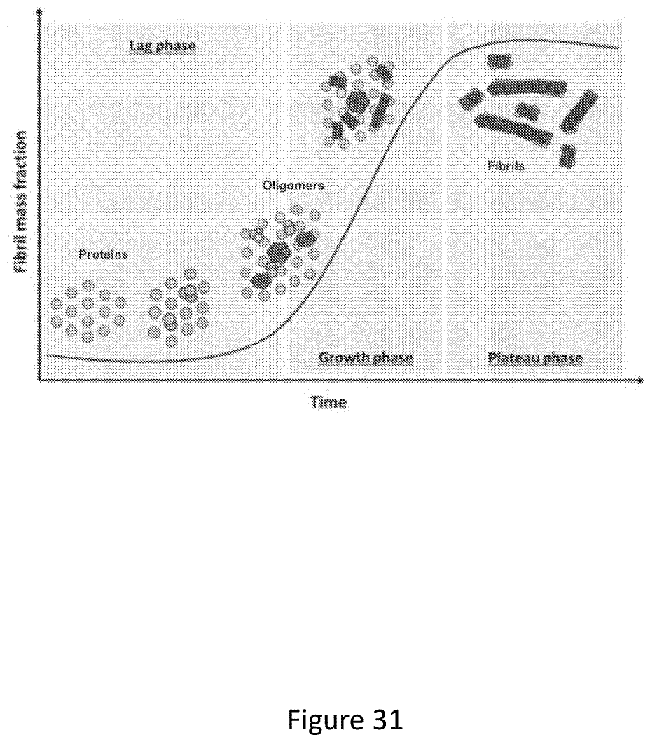

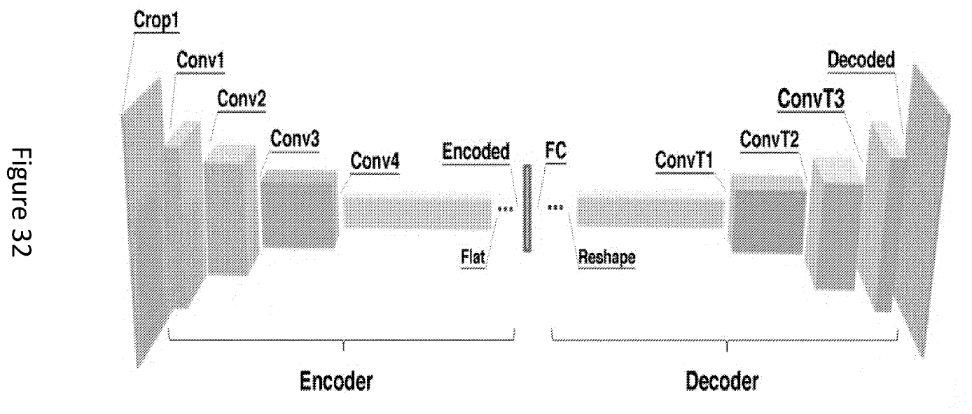

The inventors further validated the ability of AT630 to characterize pathological aggregates in situ and tested the SMLM approach on brain tissues from transgenic App^{NL-G-F} mice. These mice are genetically engineered to express mutant APP proteins that are then processed into aggregation-prone Aβ42 peptides, leading to aggregate formation in the brain. To confirm the presence of pathological aggregates in the tissue, immunostaining was performed using 6E10, an Aβ-specific antibody, together with AT630 staining. Tissues were sectioned in 10 μm slices and imaged under HILO conditions, which produced background fluorescence in both channels using diffraction-limited imaging methods (FIGS. 29A and B). Regions with amyloid structures in the tissue were appreciable by both antibody- and AT630-staining, albeit offering little information about aggregate features (FIG. 29A). To examine tissue-bound aggregates in detail, SMLM was performed to reveal the distinct aggregate structures within the amyloid regions, and demonstrated a very high degree of colocalization between AT630 and the 6E10 antibody (FIG. 29B). The percentage of colocalization was quantified by dividing the total number of colocalized signals with the total AT630 signal (see Materials and Methods) and revealed 70±20% agreement between AT630 and the antibody. Considering the increased noise level associated with tissues, the significant overlap underscores the specificity of AT630 in aggregate detection. In support of its specificity for aggregates, AT630 staining of aggregates derived from PD brain soak colocalized with anti-aS antibody (MJFR1) with 70±5% (FIGS. 29C and D). Of note is the benefit of using azimuthal or spinning TIRF image averaging to improve signal-to-background in tissue imaging (FIG. 30).

### CONCLUSION

The inventors developed an approach (exemplified using SMLM and aggregate-activated fluorophores ProteoStat and AT630) which enabled characterization of aggregates by their size and detection of structural features of small aggregates down to 10 nm in size. The data suggest that both ProteoStat and AT630 sensitively recognize features in smaller aggregate species in addition to fibrils. Combined with the observation that ThT does not seem to impede ProteoStat and AT630 staining, it is plausible that all three fluorophores recognize distinct features within aggregate structures.

The approach described herein enables quantitative detection of aggregates, and especially aggregate₄₅₀ₙₘ, as a distinct species and demonstrate their increased cytotoxicity associated with cell permeabilization. Using HEK293A cells, these invasive aggregates can be distinguished from fibrils, which are prevented from entering the cell by the plasma membrane. The data further provide evidence that membrane-permeabilizing aggregates are targeted inside the cell by proteasomes, which concentrate locally into foci, probably as part of wider mechanisms to remove harmful agents. Given their size, it is possible that aggregate₄₅₀ₙₘ may be fragments of amyloid fibrils. Since AT630 is suitable for aggregate staining in live cells, employing this aggregate-proteasome assay in conjunction with quantitative SMLM of aggregates could be a combined approach to determine the level of aggregate₄₅₀ₙₘ and their toxicity within a biological sample. Such strategies would be beneficial for early-stage disease detection of proteopathies, for example, and the difference in linear relationships between cytotoxicity and aggregate₄₅₀ₙₘ concentration (FIGS. 26C and F) enables diagnosis of specific diseases.

Importantly, data on PD and dementia with Lewy bodies suggest that a population of small as aggregates (aggregate₄₅₀ₙₘ) from different synucleinopathies may have distinct toxic effects upon cells. This could perhaps be related to specific conformations assumed by the aggregates in each pathology, and in line with the observed variations in structural conformations of αS fibrils reported for different synucleinopathies and for recombinant αS fibrils. Another possibility may be that the toxicity is proportional to the number of constituent misfolded protein monomers the individual aggregates contain. A previous study suggested no apparent correlation between the size of small aggregates and toxicity. The linear relationship between size and cytotoxicity in all samples indicate that size is a crucial factor determining membrane permeability for aggregates, while the conformations aggregates adopt may also influence toxicity once inside the cell.

The methods described herein offer a highly sensitive technique to study brain tissue, with the potential to identify cell types vulnerable to invasion by aggregates from the early stages of disease and beyond. Soluble aggregates have critical roles in propagating aggregate growth between cells, they are also faster at being internalized than fibrils, and are more likely to seed further aggregation events. Using ProteoStat and AT630, the inventors uncovered an aggregate species that is abundant at earlier stages of aggregation but was not detected by fluorophores traditionally used in imaging strategies. The inventors have further demonstrated that aggregate-activated fluorophores such as ProteoStat and AT630 may be used to quantify pathological aggregates, paving the way for future molecular studies relevant to proteopathies, e.g. neurodegenerative proteopathies.

### Materials and Methods

### Preparation and Aggregation of Misfolded Proteins

Purification of recombinant full-length tau P301S mutant (isoform 0N4R) and αS followed protocols described previously (Cliffe, R. et al. Cell Rep 26, 2140-2149 (2019) and Huang, C., Ren, G., Zhou, H. & Wang, C. C. Protein Expr Purif 42, 173-177 (2005)). Lyophilized synthetic Aβ42 (4014447, Bachem) was dissolved in PBS containing 0.5 M NaOH to make stock solution at 10 mM.

Proteins were aggregated according to standard protocols described elsewhere (De, S. et al. Nat Commun 10, 1541 (2019) and Cliffe, R. et al. Cell Rep 26, 2140-2149 (2019)).

Aggregation of 2 μM tau in PBS buffer was induced with an equimolar final concentration of low molecular weight heparin (5 kDa, Fisher Scientific) at 37° C. for 24 hrs. Aβ42 aggregation was performed at 10 μM final concentration at 37° C. in reaction buffer (50 mM Tris, 150 mM NaCl, pH 7.4). For aS aggregation, the protein was diluted to 70 μM final concentration in reaction buffer containing 0.1% NaN₃ and incubated at 37° C., 200 rpm for 72 hrs. Formation of fibrils from the aggregation reaction was verified by ThT on Cary Eclipse spectrophotometer.

### Extraction of Human Soluble Aggregates

Soluble aggregates were extracted from the brain tissues of PD and DLB donors (Table 3), following the protocol as described in Hong et al. Acta Neuropathologica 136, 19-40 (2018). The specimens were obtained from the London Neurodegenerative Diseases Brain Bank, Brains for Dementia Research, and the Multiple Sclerosis and Parkinson's Tissue Bank. Frozen temporal cortical tissues (~0.5 g) were diced and incubated with artificial cerebrospinal fluid (aCSF) (124 mM NaCl, 2.8 mM KCl, 1.25 mM NaH₂PO₄, 26 mM NaHCO₃, pH 7.4) supplemented with protease inhibitors (A32953, ThermoFisher Scientific) for 30 min at 4° C. Samples were centrifuged for 10 min at 2,000×g and the supernatant collected for subsequent ultracentrifugation at 200,000×g for 110 min at 4° C. in a SW41 Ti rotor. The upper 80% of the resulting supernatant was dialyzed against a 100-fold excess of fresh aCSF for three days with buffer changed every 24 hrs. Protein concentrations of the dialyzed samples were determined by Bradford assay, aliquoted and stored at −80° C.

TABLE 3
Details of post-mortem brain samples used.

| | | | PMDelay | Concentration | |
|---|---|---|---|---|---|
| Case | Age | Sex | (hrs) | (mg/mL) | PathDiagnosis |
| PD1 | 76 | M | 66 | 0.48 | Parkinson's Disease |
| PD2 | 85 | F | 9 | 0.44 | Parkinson's Disease |
| PD3 | 69 | F | 34 | 0.84 | Parkinson's Disease |
| DLB1 | 80 | F | 40 | 0.44 | Dementia with |
| | | | | | Lewy bodies |
| DLB2 | 71 | M | 19 | 0.60 | Dementia with |
| | | | | | Lewy bodies |
| DLB3 | 66 | M | 53 | 0.75 | Dementia with |
| | | | | | Lewy bodies |

### Mouse Tissue Staining

Animal experiments were conducted according to the United Kingdom Animals (Scientific 643 Procedures) Act 1986. Brain hemispheres were flash frozen from adult (P240) App^{NL-G-F} homozygous mice harboring the Swedish (KM670/671 N L), Beyreuther/Iberian (1716 F) and Arctic (E22G) mutations in the APP gene on a C57BL/J6 background. Animals were transcardially perfused with 20 ml of ice-cold (4° C.) oxygenated dissection artificial cerebrospinal fluid (aCSF) (108 mM choline-Cl, 3 mM KCl, 26 mM NaHCO₃, 1.25 mM NaHPO4, 25 mM D-glucose, 3 mM Na pyruvate, 2 mM CaCl₂) and 1 mM MgSO₄ saturated with 95% O₂/5% CO₂). OCT embedding matrix was used to fix hemispheres, which were subsequently sectioned in 10 μm slices on a cryostat at −20° C. Before staining, each section was repeatedly washed in PBS to remove the OCT, followed by incubation with PBS containing 4% paraformaldehyde, 0.3% Triton-X100. The same buffer was used for blocking with goat serum prior to staining with primary anti-Aβ antibody 6E10 overnight. Secondary Alexa488-tagged antibodies were used for staining the next day followed by AT630 staining, immediately before super-resolution imaging.

### Cytotoxicity Assay

Toxicity of aggregates were determined with lactate dehydrogenase (LDH) assay (Kaja, S. et al. J Pharmacol Toxicol Methods 73, 1-6 (2015)). The procedure for aggregate measurement has been described previously (Cliffe, R. et al. Cell Rep 26, 2140-2149 (2019)). Aggregate samples or control buffers were added in triplicates to 200 μl containing 30,000 HEK293A cells per well in 48-well plates. Lysis buffer was added 45 mins before the end of the experiment to the maximum LDH activity set of control cells. After 4 or 24 hrs incubation at 37° C., media was removed and centrifuged at 200×g for 5 min. 50 μl media was incubated with 50 μl of the LDH assay buffer for 1 hr and the reactions were quenched with 50 μl 1 M acetic acid. Absorbance at 490 nm and 680 nm were measured for each reaction on a plate reader.

### Fluorophore Staining and Imaging

ProteoStat (ENZ-51023, Enzo Lifesciences) or Amytracker 630 (AT630, EBBA Biotech) were diluted 1:50 in PBS for stock aliquots and stored at −20° C. This stock was further diluted 1:50 (ProteoStat) or 1:1000 (AT630) for final single-aggregate TIRF imaging. Single-aggregate TIRF imaging was performed as described previously (Cliffe, R. et al. Cell Rep 26, 2140-2149 (2019)). Briefly, aggregates were diluted to 700 nM (αS), 400 nM (tau) or 300 nM (Aβ) imaging concentrations with indicated fluorophores and incubated on a coverslip for 15 min. Samples were imaged on a TIRF microscope. For super-resolution microscopy, samples were incubated in freshly prepared imaging buffer (PBS with 1 mg/ml glucose oxidase, 0.02 mg/ml catalase, 10% (w/w) glucose, and 100 mM methylamine (MEA)) immediately prior to imaging.

The generation of the HEK293A cells expressing eGFP tagged PSMD14 subunit is described in detail by Zhang et al. bioRxiv, 487702, doi: 10.1101/487702 (2018). Live cell imaging of these cells incubated with 1 μM aggregates was conducted in FluoroBrite DMEM (ThermoFisher Scientific) supplemented with 10% fetal bovine serum.

Imaging with TIRF Microscope

Samples were imaged using an ECLIPSE Ti2-E inverted microscope (Nikon). Lasers were housed in a C-FLEX laser combiner (HÜBNER Photonics), containing 405 nm (06 series, HÜBNER Photonics), 488 nm (06 series, HÜBNER Photonics), 561 nm (04 series HÜBNER Photonics) and 638 nm (06 series, HÜBNER Photonics). These were all aligned inside the combiner and were coupled to the E-TIRF arm (Nikon). A filter cube containing a dichroic mirror, in addition to bandpass and longpass filters for all four laser lines (C-NSTORM QUAD 405/488/561/647 FILT, Nikon) was installed inside a motorized filter turret below a CFI Apochromat TIRF 100XC NA 1.49 Oil objective (Nikon). Samples were placed on a motorized stage, and a Perfect Focusing System (Nikon) was used to minimize drift in the z-direction. Images were recorded by a sCMOS camera (Prime95B, Photometrics) with a pixel size of 11×11 μm, at 20 Hz. Super-resolution (single-molecule localization microscopy; SMLM) images were reconstructed from 2000 frames, and diffraction-limited images of aggregates on coverslips were averaged from 100 frames. In order to record axial information of single molecules, a cylindrical lens (f=1000.0 mm, Plano-Convex, Thorlabs) was placed in the Optosplit II, and followed the astigmatism method of 3D SMLM (Huang, B., Wang, W., Bates, M. & Zhuang, X. Science 319, 810-813, (2008)).

### SMLM Image Reconstruction

SMLM images in 2D were reconstructed using Matlab scripts described in detail by Yin et al. Nat Commun 10, 119, (2019). Briefly, each frame was initially filtered using a box filter, set with a box four times the width of 2D Gaussian point spread function (PSF), and each pixel intensity was weighted by the inverse of its variation. Local maxima were then recorded and fitted to a 2D-Gaussian single-PSF using the Maximum Likelihood Estimation (MLE) algorithm performed in GPU (Nvidia GTX 1060, CUDA 8.0). Alignment of two colors was achieved by recording a map of chromatic aberrations using Tetraspeck beads (0.1 μm, fluorescent blue/green/orange/dark red; Life Technologies) that fluoresce across a broad range of wavelengths and readily adhere to a plasma-cleaned coverslip. The centers of these beads were recorded upon excitation of the 488 nm, 561 nm and 638 nm lasers, and a second order polynomial function was used to correct for the chromatic aberration across the three colors. For the calculation of localization precision, each burst of fluorescent from individual fluorophores was aligned relative to each other, and an average PSF was calculated. Calibration of the sCMOS camera enabled the offset for each pixel to be calculated and allows the number of photons to be calculated for each burst (Yin, Y., Lee, W. T. C. & Rothenberg, E. Nat Commun 10, 119, (2019)). This was used to define the localization precision using Δloc=FWHM\sqrt{photons}.

Recorded 3D images were reconstructed using ThunderSTORM (Ovesny, M., Krizek, P., Borkovec, J., Svindrych, Z. & Hagen, G. M. Bioinformatics 30, 2389-2390 (2014)). Tetraspeck beads deposited on a plasma-cleaned coverslip were used, and the beads were imaged whilst scanning along the z-axis, with the cylindrical lens installed in the Optoslipt II. These images were used to produce reference curves that were used to calculate the axial position of the fluorophores bound to aggregates during 3D SMLM experiments. Reconstruction of 3D models from SMLM were made by fitting the recorded images with the elliptical Gaussian PSF (astigmatism) model using the weighted least-squares method. Following the methodology described et by Huang al. Science 319, 810-813, doi: 10.1126/science.1153529 (2008), αS monomers that were singly-labelled with Alexa647 were deposited on a plasma cleaned coverslip. The relative 2D and 3D positions measured within 100 nm from the average focal plane were plotted for each fluorophore across individual blinking cycles. This gave clusters of localizations which could be fitted to Gaussian curves to quantify our localization accuracy (FIG. 16). SMLM in 2D gave Gaussians with a full width half maximum (FWHM) of 27 nm and 28 nm for both x and y directions. Adding the cylindrical lens resulted in Gaussians of 33 nm, 42 nm and 84 nm for x, y and z directions, respectively.

### Quantitative Analysis of Imaging Data

The images produced from the SMLM and diffraction-limited TIRF microscopy experiments were analyzed using custom written Matlab codes to quantify the lengths (sizes) and total intensities of each aggregate, previously described in detail by Cliffe et al. Cell Rep 26, 2140-2149 (2019). Briefly, 100 frames were averaged, and the averaged images were top-hat and bpass filtered to reduce noise from the camera and subtract background. The resultant images were then blurred using a Gaussian filter before calculating the outline of the individual aggregates. The boundaries were thinned to determine the size of each particle, and a signal-to-background correction for each pixel was used to measure the total intensities.

To identify aggregates that were internalized by cells, the whole cell was imaged using stacks of images 100 nm apart in the z-direction. These cells expressed GFP-tagged proteasomes, and the cell boundaries from each stack were determined using a rolling ball filter on the images from the GFP emission (Zhang, Y. et al. bioRxiv, 487702, doi: 10.1101/487702 (2018)). Aggregates that were found outside these boundaries were then masked from the stack of images and excluded from further analysis. The pixels were then summed in the z-projection across the stack of images, and these resulting 2D images were put through the size analysis described above.

Image analysis for colocalization experiments was performed using the Colocalization Threshold plug-in in ImageJ software. This analysis gave the thresholded Manders' coefficients for each channel. The Colocalization Threshold plug-in uses the Costes method to automatically determine the thresholds for the two channels (Costes, S. V. et al. Biophys J 86, 3993-4003 (2004)).

### Statistical Calculations

All uncertainties are presented to one significant Figure only, as the second significant Figure for each uncertainty will be within the error reported by the first. Similarly, the accuracy of all quantities is reported within its corresponding error; therefore the last significant Figure of each measurement is the same Figure as the first significant Figure in the corresponding uncertainty.

N numbers for the number of aggregates and cells imaged from each experiment are found in the corresponding Figure legends. A Kolmogorov-Smirnov test was used in FIG. 12D to demonstrate AT630 and ProteoStat produced a distribution of images that were statistically different to those observed using ThT (p<0.0005). Scatter plots in FIGS. 21, 23, 26 and 29 are reported with the mean values and standard error of mean (SEM) of the aggregate sizes for each aggregate sample. The crossover at 450+60 nm for the 3 hrs and 24 hrs samples shown in FIG. 21B was calculated by fitting each distribution to a single exponential decay using equation 1, where x is the aggregate size, A is the amplitude and 1/t₁ represents the decay rate.

$$y = A {e}^{[ - x / {t}_{1} ]} \qquad Equation 1$$

The error reported at the crossover is the square root of the sum of the SEM for t1 of the two fitted curves. Similarly, the 21±3 nm shift in FIG. 21D was calculated by subtracting the median value of each curve, and the error is the square root of the sum of the SEM of t1 the two fitted curves. Error bars in the inset in FIG. 21D represent the standard deviation of the mean number of aggregates in an individual cell. Statistical significance between size groups at different time points was calculated using a two sample t-test with n.s. (no significance) p>0.05, *p<0.05 and ** p<0.005. Cytotoxicity values in FIGS. 25-27 and 29 are reported as mean and standard deviation of triplicate experiments.

### Calculation of Photophysical Properties

The quantum yield (Φ) and extinction coefficient (ε_{A}) of Alexa488, Alexa568, Alexa647 and ThT have been previously validated by other research groups and the dyes' manufacturers. The precise properties of ProteoStat and AT630 are protected due to proprietary reasons. The manual from Enzo Life Sciences states that ProteoStat is approximately 3 μM at the recommended final concentration, and this was used to calculate an extinction coefficient from the UV-vis absorption spectrum. Ebba Biotech, who produce the Amytracker dyes, were able to inform us that the average molecular weight across the range of Amytracker dyes was 660±30 g/mol. The user manual from Ebba Biotech also states that the concentration of AT630 is 1 mg/ml, and this was then used to calculate the molar concentration of AT630 and enabled us to calculate the extinction coefficient from the UV-vis absorption spectrum. The excitation and emission profile of Alexa568 is similar to both ProteoStat and AT630, so an anti-rabbit secondary antibody-labelled with Alexa568 (Invitrogen, A11011) was used as reference to calculate the relative quantum yields of ProteoStat and AT630. The emission spectra from a range of concentrations of the fluorophores absorbances ranging from 0.1-0.9 AU were measured in the presence of an excess of αS fibrils (10 μM). The emission profiles were integrated (I) and plotted against the absorbance (A) and were fitted to a straight line. The gradients of each linear plot were compared to the Alexa568, using equation 2 described by Wong et al. J Lumin 224, doi: ARTN 117256 using the quantum yield of Alexa568 (Φ_{R}=0.69) as a reference. The brightness of each dye was calculated simply using Brightness=Φ×ε_{A}.

$${Φ}_{S} = {Φ}_{R} ( \frac{{I}_{S}}{{I}_{R}} ) ( \frac{1 - {10}^{- {A}_{R}}}{1 - {10}^{- {A}_{S}}} ) {( \frac{{n}_{S}}{{n}_{R}} )}^{2} \qquad Equation 2$$

where n=refractive index of the solution.

### Example 4—Dimensional Reduction, Autoencoders and Principal Component Analysis

Described herein is a method of classifying protein aggregates using an autoencoder and principal component analysis (PCA). The methods can be used to define aggregate morphology from single-molecule imaging of protein aggregates from brain region samples and cerebrospinal fluid (CSF) samples.

### Dimensional Reduction

As described above, neural networks can be used to classify a proteopathy in a patient based on the morphology of small protein aggregates in a patient sample. However, protein aggregate morphologies can also be classified using dimensionality reduction methods to identify aggregate subpopulations associated with each disease.

### Convolutional Autoencoder Model Parameters and Training

The exemplary CAE architecture illustrated in FIG. 32 can be used to classify images of protein aggregates. The architecture comprises convolutional and linear layers. In the convolutional segment, the model parameters include input and output channels, kernel size, stride, and padding. The convolutional layer sequence has input-output channel mappings of 1-8, 8-16, and 16-32, each using a kernel size of 3 (the size of a 3×3 filter that moves over the input data), a stride of 2 (the filter jumps two pixels/units at a time, reducing the spatial dimensions of the output by approximately half compared to the input), and a padding of 1 (used to add extra pixels/units around the border of the input data, to control the spatial dimensions of the output feature maps, and helping to mitigate against the spatial reduction caused by the stride of 2). Linear layers are defined by the number of input and output features, with one layer configured for handling 32×32×32 input features and outputting 128 features. However, it will be appreciated that any other suitable configuration of the layers of the autoencoder could alternatively be used. In the present example, the one layer takes an input with 1 channel and produces an output with 8 channels. The next layer takes the 8-channel output from the previous layer and transforms it into an output with 16 channels. The next layer takes the 16-channel output from the previous layer and produces an output with 32 channels.

Hyperparameters guiding the training process include a learning rate (LR) set at 0.001, which undergoes a decay factor (that reduces the learning rate) of 0.1. However, it will be appreciated that other suitable learning rates and decay factors could alternatively be used. The model employs a batch size of 64 samples for each iteration, with a mean squared error (MSE) loss function to measure the discrepancy between predicted and actual values. The training procedure incorporates a 20% validation split (in other words, 80% of the data is used for training the model, and 20% of the data was used to validate the model) to gauge the model's generalization capability on unseen data. An optimization technique can be used that operates on first-order gradients (the algorithm utilises the first derivatives of the objective function, typically the loss function in the context of neural networks) of stochastic objectives, adapting based on lower-order moment estimates. 500 epochs and a tolerance of 10 (if the validation loss does not improve for 10 consecutive epochs, the training process would be halted) may be used to train the model. The model from the epoch with the best validation performance is saved and used for future predictions. The training computations may be performed using CUDA-compatible graphics processing unit (GPU), but could alternatively be performed using a central processing unit (CPU), or any other suitable apparatus.

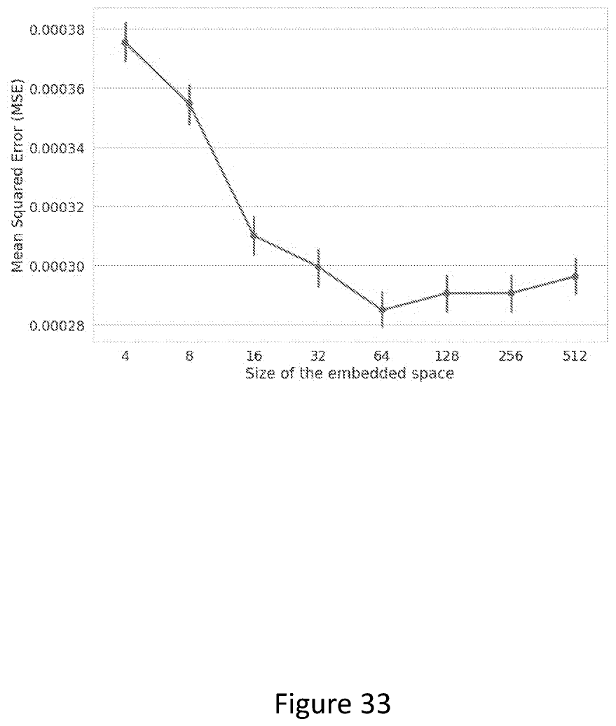

The model was trained using embedding sizes of: 4, 8, 16, 32 64, 128, 256, and 512 values (the size of the lower dimensional representation in the latent space). The primary metric used for performance evaluation was the Mean Squared Error (MSE), illustrated in FIG. 33 for each embedding size for the brain soak samples. The x-axis corresponds to the various sizes used for the embedded space, and the y-axis corresponds to the mean squared error (for the output images with respect to the input images). The mean squared error (MSE) between the original image (xi) and the reconstructed image (1) is calculated using the following equation:

$$M S E = \frac{1}{N} {\sum }_{i = 1}^{N} {( {x}_{i} - \text{?} )}^{2} ,$$

where N is the total number of pixels, xi represents the pixel value in the original image and \hat{x}ₗ represents the pixel value in the reconstructed image.

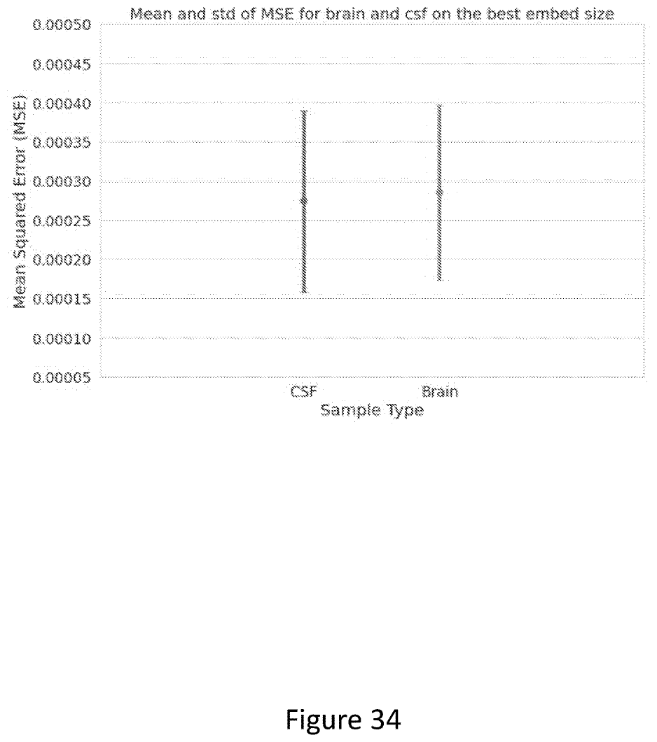

Use of an embedding size of 64 resulted in the lowest MSE among all other tested embedding sizes. This reflects the balance between ensuring sufficient data compression without a significant loss of salient information in the lower dimensional representation, or overfitting. Aggregates from the CSF and brain regions contain similar shape attributes and thus, the same autoencoder to reconstruct images of the aggregates was tested if was applicable for both regions. The autoencoder trained using images of aggregates from the brain region was applied to images of aggregates from the CSF. FIG. 34 illustrates a comparison of the autoencoder's reconstruction error, measured by the MSE, using images of aggregates from the brain and CSF.

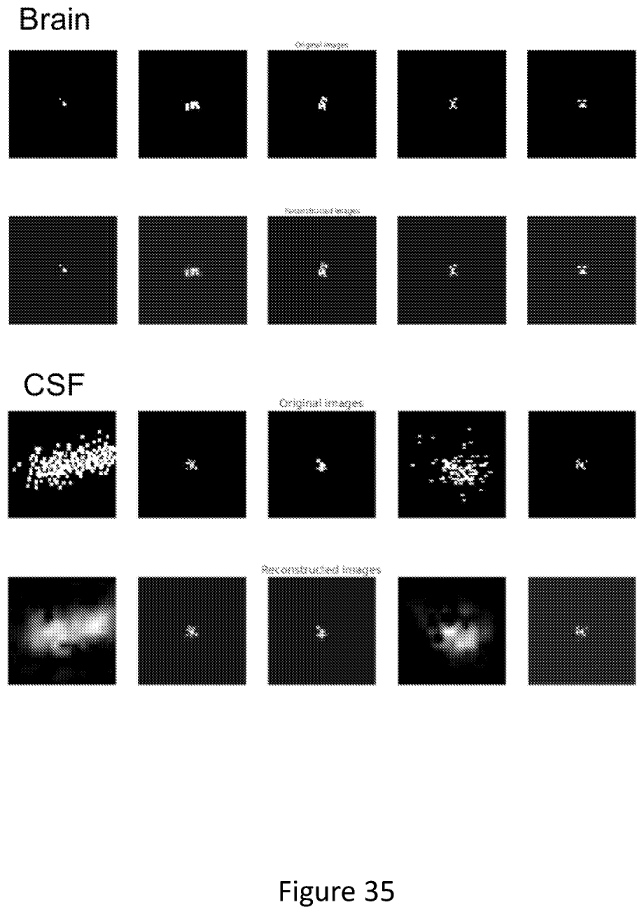

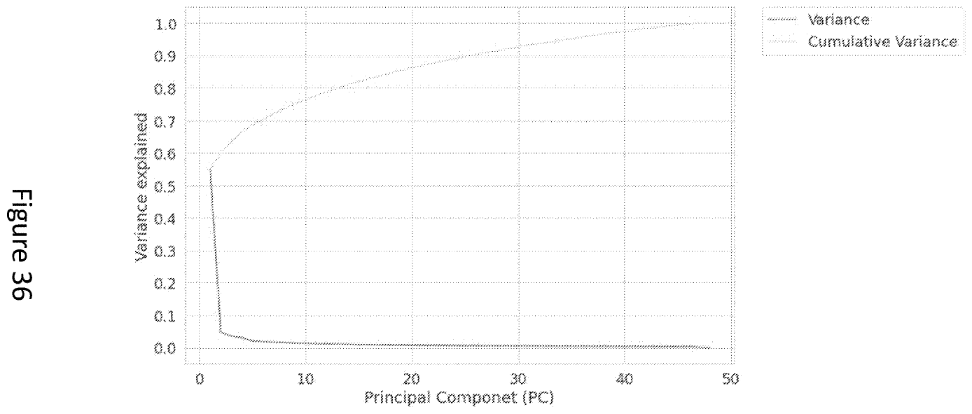

Notably, the autoencoder's performance remained consistent and the model performed well at image reconstruction using the CSF images even when trained using images of aggregates from the brain. FIG. 35 shows examples of images of protein aggregates reconstructed using the convolutional autoencoder, including examples from both brain region aggregates and CSF region aggregates, using a 64-value embedding size.

### Principal Component Analysis (PCA) and Nearest Neighbour Search (NNS)

Advantageously, the inventors have found that the combination of PCA with the use of a convolutional autoencoder provides detailed information regarding the cell aggregate structures, and enables an understanding of cell aggregate shapes in cell imaging data and their relationship to disease. These techniques provide a comprehensive approach, leveraging both the density distribution and inherent similarities among the data, enabling precise and reliable disease classification. Whilst in the examples below PCA is performed on the output of the autoencoder, it will be appreciated that an autoencoder need not necessarily be used. Alternatively, for example, PCA could be performed directly on the processed images of the protein aggregates, and the resultant PCA values could be input into any suitable classification algorithm. In the present example, PCA components were determined that captured 99.99% of the total variance in the data. The PCA variables are generated by performing eigen-decompositions, sorting, transformation and selecting components. The method comprises looking for directions (eigenvectors) in the data for which there is the most change or ‘spread’. After finding these directions, they are ranked based on importance (variance), with the most important direction corresponding to PC1 (principal component 1). The data is then reshaped based on the directions, maintaining 99.99% of the information but using fewer dimensions.

Test data from brain soak samples and cerebrospinal fluid samples used to perform classification using the autoencoder, PCA and NNS are illustrated in Tables 4 and 5 respectively, below. The tables illustrate categorisation of subjects into distinct classes and their corresponding counts. The brain soak data of table 4 comprises five categories: control, PD, LDB, FTD and AD. The cerebrospinal fluid data of table 5 comprises three categories: control, PD and AD. The “counts by class” indicated the total number of aggregate images in each of these classes. Each primary class is further subdivided by patient, and the corresponding counts are shown under “counts by patient”. A total of 83,992 images from the brain soak samples data were used, and 13,312 images from the cerebrospinal fluid samples data were used.

TABLE 4
Brain soak data input into the trained autoencoder.

| Class | Counts by Class | Patients | Counts by Patient |
|---|---|---|---|
| Control | 14949 | Control 1 | 11220 |
| | | Control 2 | 3729 |
| PD | 22440 | PD 1 | 11220 |
| | | PD 2 | 11220 |
| DLB | 11220 | DLB 1 | 11220 |
| FTD | 22440 | FTD 1 | 11220 |
| | | FTD 2 | 11220 |
| AD | 12943 | AD 1 | 8992 |
| | | AD 2 | 3951 |

TABLE 5
Cerebrospinal fluid data input into the trained autoencoder

| Class | Counts by Class | Patients | Counts by Patient |
|---|---|---|---|
| Control | 5706 | Control 1 | 2853 |
| | | Control 2 | 2853 |
| PD | 4753 | PD 1 | 2853 |
| | | PD 2 | 1900 |
| AD | 2853 | AD 1 | 2853 |

Processed images of protein aggregates from samples were input into the trained autoencoder, and PCA was performed on the autoencoder output (the lower dimensional embedded space).

FIG. 36 shows a variance plot that was generated using PCA on the embedded representations generated using the autoencoder from the images of protein aggregates. The x-axis corresponds to the principal components, ordered by the amount of variance they capture from the original embeddings. The y-axis corresponds to the individual and cumulative variance corresponding to the principal components, illustrating the proportion of the total variability in the 64-dimensional space captured by each successive component. As illustrated in the figure, the first principal component captured 55% of the total variance, while the cumulative variance of the first five principal components accounted for approximately 70% of the overall variance. A significant revelation from this plot was the pronounced dominance of the initial principal components in capturing the majority of the variance in the dataset. The first principal component stood out prominently, capturing a substantial 55% of the total variance. In contrast, the second principal component, represented 5% of the total variance. The significant difference in variance contribution between the two components suggests that the first component captures the largest variations within the dataset.

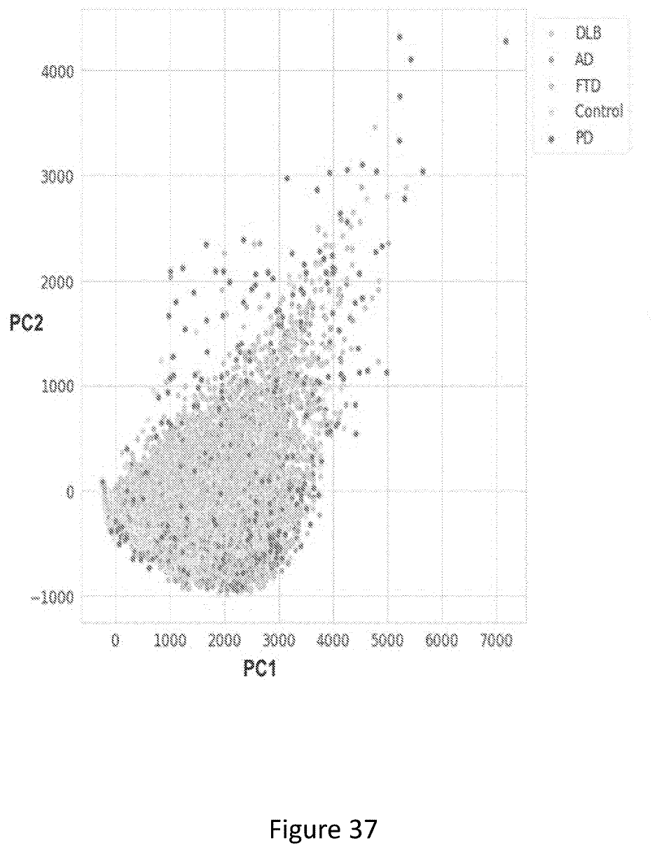

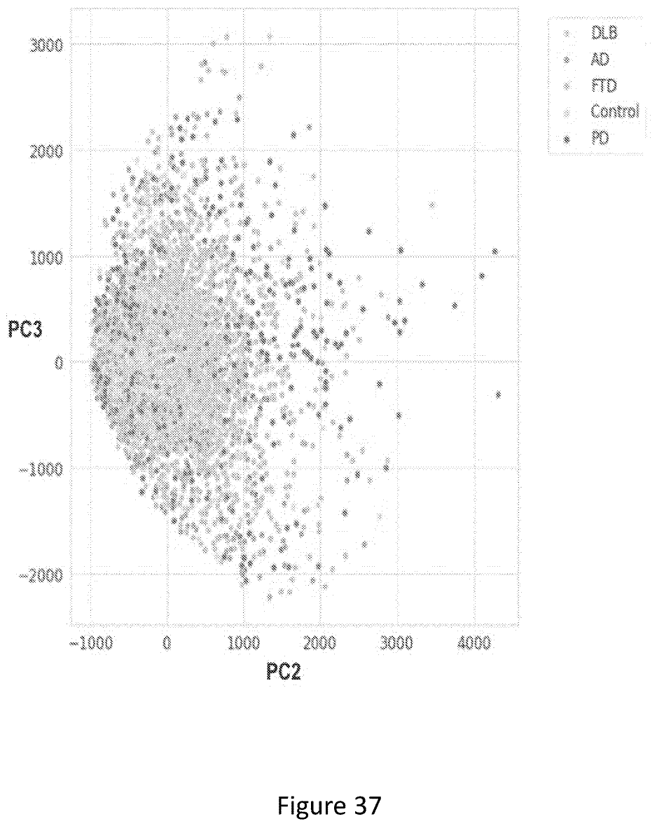

FIG. 37 shows scatter plots illustrating the relationships between the principal components derived from the embedded space output from the autoencoder using images of protein aggregates from the brain samples. The left-hand plot shows the first principal component (PC1) plotted against the second principal component (PC2), while the right-hand plot shows the second principal component (PC2) plotted against the third principal component (PC3). Each datapoint is labelled based on disease class: Alzheimer's Disease (AD), Parkinson's Disease (PD), Frontotemporal Dementia (FTD), Dementia with

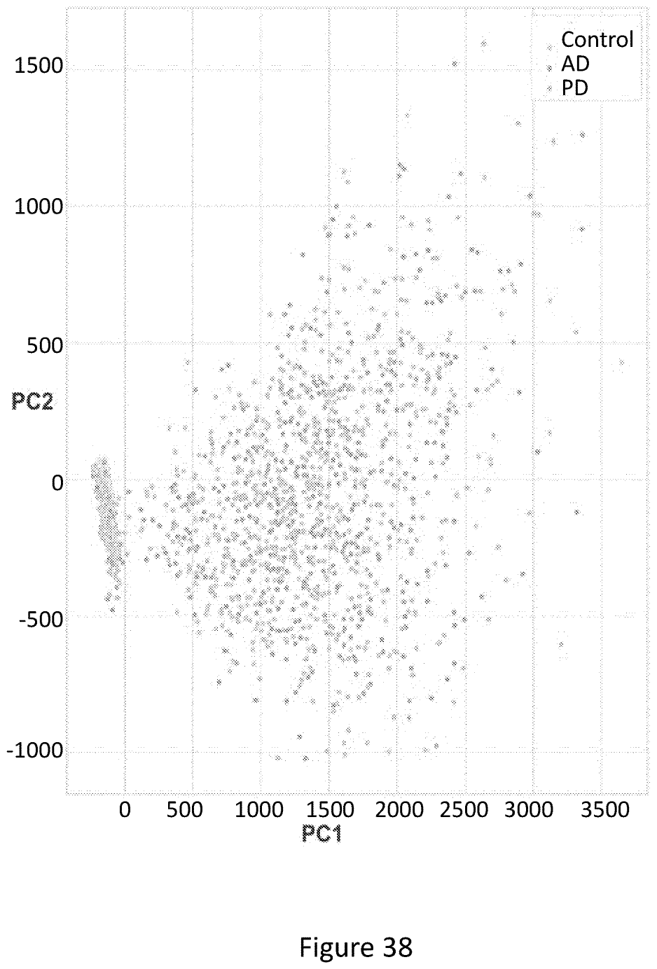

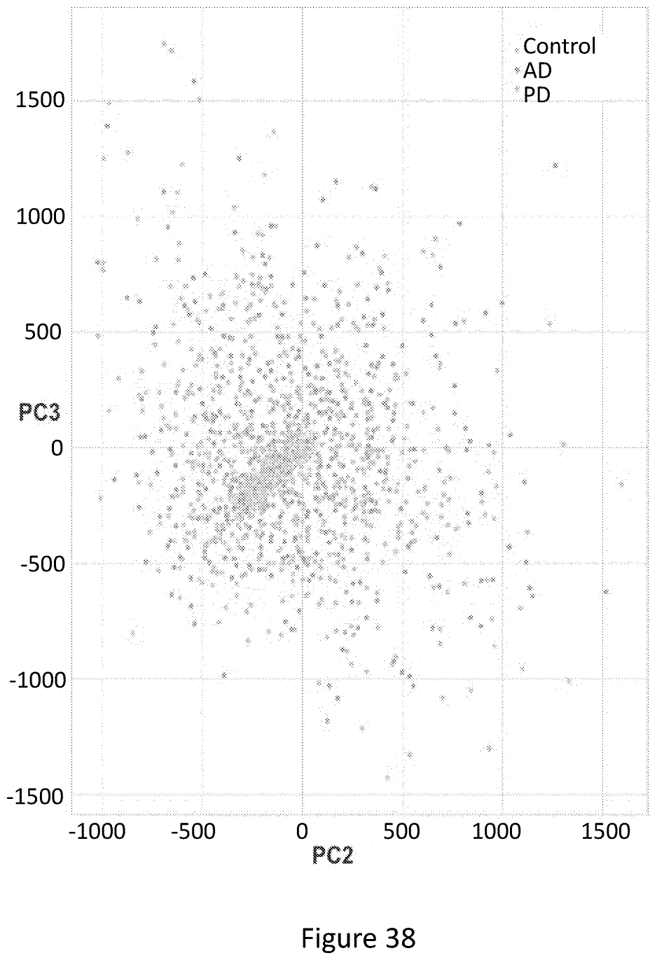

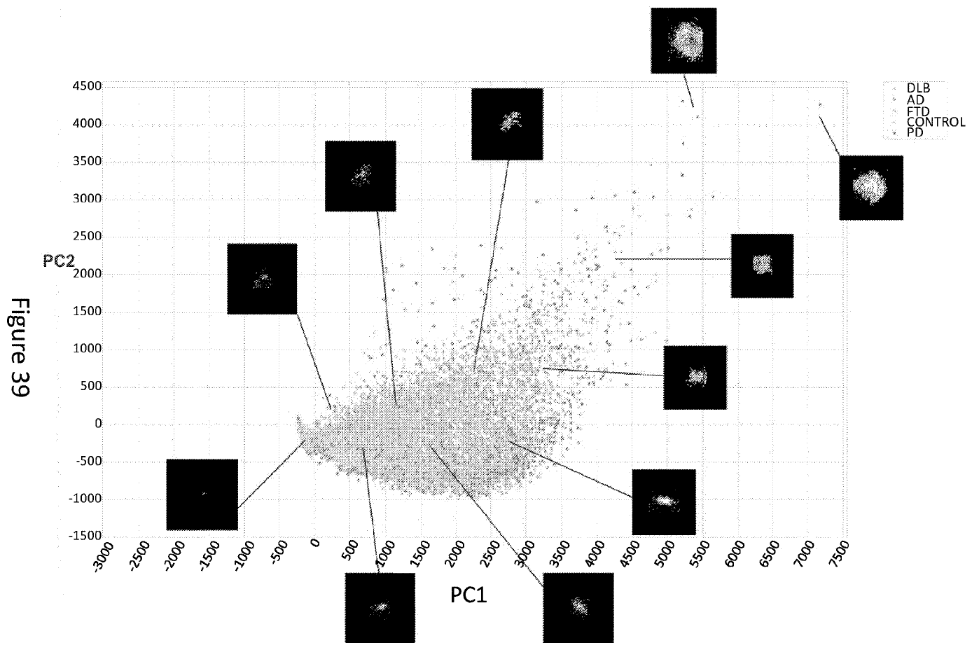

Lewy Bodies (DLB), and Control. The spatial distribution and clustering of the data points in FIG. 37 shows patterns or groupings of the data. FIG. 38 shows Scatter plots illustrating the relationships between the principal components derived from the images of protein aggregates from the CSF samples. The data points from the CSF samples are more dispersed than the brain samples.

FIG. 39 shows a scatter plot illustrating the relationship between PC1 and PC2 (for brain sample aggregates). By tracing back specific data points to the original images, visual patterns emerge in aggregate shapes. Moving from left to right in the scatter plot is associated with changes in the aggregate shape, as is the transition from top to bottom. These images provide a tangible context to the principal component dispersion. From the left to the right of the plot there is a noticeable increase in the cell aggregate size. This suggests that the x-axis, represented by PC1 on the plot, is indicative of some size metric or related feature within the dataset. The cell aggregates appeared more dispersed towards the top of the plot, whereas, at the bottom of the plot the aggregates appear more concentrated or clustered. This spatial distribution indicates a potential gradient or variation in cell aggregate density or dispersal patterns across the dataset.

### Heatmap Analysis

Heatmap analyses provide additional insight into the intrinsic patterns within the data. Notably, strong positive correlations between PC1 (the first principal component) and specific features such as branch numbers, skeleton size, and area were found. Using the PCA coordinates, a density plot was constructed using the first and second principal components to visualize the densities of each class. Control data was used to determine a gaussian distribution and to set a threshold of the mean plus two times the standard deviation for the control group. NNS enables identification of the significant aggregate areas and centroids of the aggregates, and can be used to generate a heat map to visualize the variations in PCA dimensions, highlighting the patterns in the PCA that correspond to each disease type.

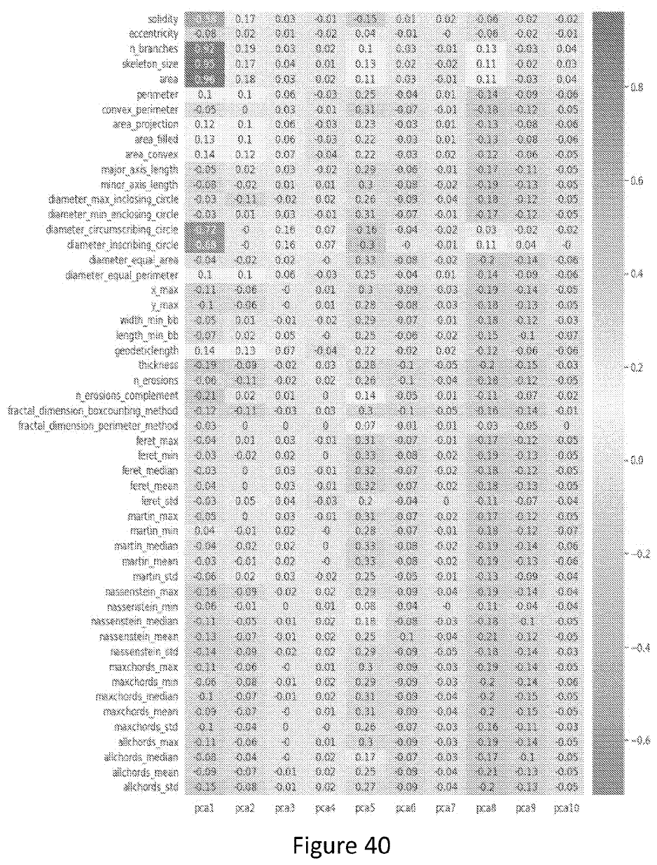

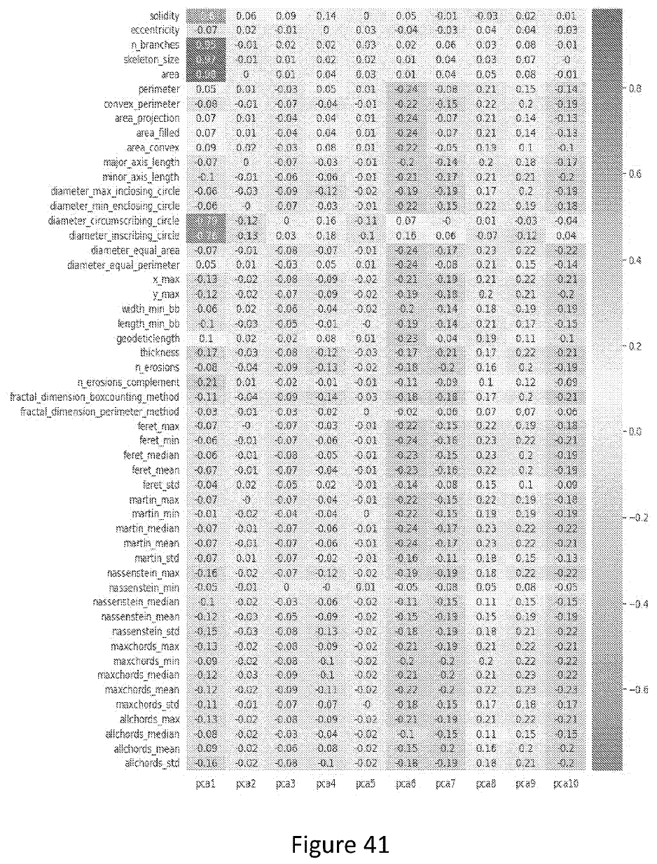

FIG. 40 shows a heatmap illustrating correlations between the principal components and various features within the brain sample data, and FIG. 41 shows the same heatmap but for the CSF data. It will be appreciated that the morphological features of each protein aggregate may be extracted using any suitable image processing method (e.g. using any suitable image recognition software), such as OpenCV version 4.5.5 and/or Scikit-image version 0.19.2, or the iMEA package. FIGS. 40 and 41 show the Pearson correlation coefficient for each shape feature of the protein aggregates, representing the linear relationships between each of the first ten principal components derived from the convolutional autoencoder embeddings and the aggregate shape features. The shading indicates the strength and direction of the correlation. A value of 1 corresponds to a perfect positive linear relationship, and a value of −1 corresponds to a perfect negative linear correlation. A value of 0 corresponds to no linear correlation. For two variables, X and Y, with n data points each, the Pearson correlation coefficient r is given by:

$$r = \frac{{\sum }_{i = 1}^{n} ( {x}_{i} - \overline{x} ) ( {y}_{i} - \overline{y} )}{\sqrt{{\sum }_{i = 1}^{n} {( {x}_{i} - \overline{x} )}^{2}} \sqrt{{\sum }_{i = 1}^{n} {( {y}_{i} - \overline{y} )}^{2}}}$$

where xᵢ and yᵢ are individual data points, and x and y are the means of x and y respectively.

Shape features from FIGS. 40 and 41 will now be described. The solidity is the solidity of the aggregate. The eccentricity is the eccentricity of the aggregate. ‘n_branches’ is the number of branches of the aggregate. ‘Skeleton_size’ is the skeleton size for the aggregate. ‘Area’ is the area of the aggregate. Perimeter is the perimeter of the protein aggregate. ‘Convex_perimeter’ is the perimeter of the convex hull. ‘Area_projection’ is the projection area. ‘Area_filled’ is the filled projection area. ‘Area_convex’ is the area of the convex hull. ‘Major_axis_length’ is the major axis length of the legendre ellipse of inertia (ellipse that has the same normalized second central moments as the particle shape). ‘Minor_axis_length’ is the minor axis length of the legendre ellipse of inertia. ‘Diameter_max_inclosing_circle’ is the diameter of the maximum incircle of the projection area. ‘Diameter_min_enclosing_circle’ is the diameter of the minimum circumference of the projection area. ‘Diameter_circumscribing_circle’ is the diameter of the circumcircle with the same centre as the particle contour and maximum area, which touches the particle contour from the inside. ‘Diameter_inscribing_circle’ is the diameter of the circumcircle with the same centre as the particle contour and minimum area, which touches the particle contour from the outside. ‘Diameter_equal_area’ is the diameter of a circle of equal projection area. ‘Diameter_equal_perimeter’ is the diameter of a circle of equal perimeter. ‘X_max’ is the maximum longest chord. ‘Y_max’ is the longest chord orthogonal to X_max. ‘Width_min_bb’ is the width of the minimal 2D bounding box. ‘Length_min_bb’ is the length of the minimal 2D bounding box. ‘Geodeticlength’ is the geodetic length of the protein aggregate. ‘Thickness’ is the thickness of the aggregate. ‘N_erosions’ is the number of pixel erosions to completely erase the silhouette of a particle in the binary image. ‘N_erosions_complement’ is the number of pixel erosions to completely erase the complement between convex hull and object. ‘Fractal_dimension_boxcounting_method’ is the fractal dimension determined by the box counting method. ‘Fractal_dimension_perimeter_method’ is the fractal dimension determined by the perimeter method. ‘Feret_max’ is the maximum ferret diameter of the aggregate (the Feret diameter is a measure of an object's size along a specified direction). ‘Feret_min’ is the minimum ferret diameter of the aggregate. ‘Feret_mediam’ is the median of all Feret diameters. ‘Feret_mean’ is the arithmetic mean of all Feret diameters. ‘Feret_std’ is the standard deviation of all Feret diameters. ‘Martin_max’ is the maximum Martin diameter (the Martin diameter is the length of the area bisector of an irregular object in a specified direction of measurement). ‘Martin_min’ is the minimum Martin diameter. ‘Martin_median’ is the median of all Martin diameters. ‘Martin_median’ is the median of all Martin diameters. ‘Martin_mean’ is the arithmetic mean of all Martin diameters. ‘Martin_std’ is the standard deviation of all Martin diameters. ‘Nassenstein_max’ is the maximum Nassenstein diameter. ‘Nassenstein_min’ is the minimum Nassenstein diameter. ‘Nassenstein_median’ is the median of all Nassenstein diameters. ‘Nassenstein_mean’ is the arithmetic mean of all Nassenstein diameters. ‘Nassenstein_std’ is the standard deviation of all Nassenstein diameters. ‘maxchords_max’ is the maximum of max chords (max chord is the maximum of all chords for one particle rotation). ‘maxchords_min’ is the minimum of max chords. ‘Maxchords_median’ is the median of max chords. ‘Maxchords_mean’ is the mean of max chords. ‘Maxchords_std’ is the standard deviation of max chords. ‘Allchords_max’ is the maximum of all chords for all rotations. ‘Allchords_median’ is the median of all chords for all rotations. ‘Allchords_mean’ is the mean of all chords for all rotations. ‘Allchords_std’ is the standard deviation of all chords for all rotations.

For the brain samples, a standout observation was the compelling positive correlation of PC1 with three primary features: number of branches (0.92), skeleton size (0.95), and area (0.96). Such robust positive correlations imply a direct relationship: as the value of these features ascends, the PC1 value correspondingly rises. If the number of branches signifies the count of branches in a given structure, a higher branch count typically corresponds to a larger skeleton size and greater area. Structures with more branches tend to be more expansive and complex. This suggests that PC1 effectively captured this variation and highlights the size and intricacy of the studied structures.

PC1 demonstrated negative correlations with the features solidity (−0.58), diameter circumscribing circle (−0.72), and diameter equal area (−0.68). These negative correlations infer an inverse relationship. Consequently, an increase in these feature values leads to a decrease in the PC1 value. Solidity, in geometric contexts, gauges a shape's compactness. Hence, a diminished solidity index points towards a shape replete with cavities or irregularities. This negative association insinuates that structures exhibiting lower solidity-those that are more irregular or possess pronounced voids-register elevated PC1 values. Moreover, the observed negative associations with diameter features suggest that structures boasting expansive diameters or overarching shapes correlate with diminished PC1 values. The relationships between structure size/complexity and solidity provide insight into the intricacies of the aggregate structures, highlighting length as a significant factor in pathological aggregate structure.

Overall, PC1 represents the balance between a structure's size and complexity versus its solidity. Structures with more branches, larger skeletons, and bigger areas tend to have higher PC1 values. On the other hand, more solid structures or those with wider diameters tend to have lower PC1 scores.

PC2 predominantly displays negative correlations with the features, with most values greater than −0.1. This suggests that as the values of certain features decrease, the PC2 score tends to increase, but this relationship is not as pronounced as one might observe with stronger correlations. It can be inferred that PC2 may be capturing the inverse trends of certain features without being heavily influenced by any single attribute.

In contrast to PC1 and PC2, PC5 largely exhibits positive correlations with the features. However, these correlations are relatively mild, with most values being less than 0.5. This suggests a moderate direct relationship between the rise of certain features and the increase in PC5 values. PC5 appears to capture the direct trends of these features, emphasizing attributes that align positively with it, but without strong dominance from any single feature. These trends apply to both the brain samples and the CSF samples.

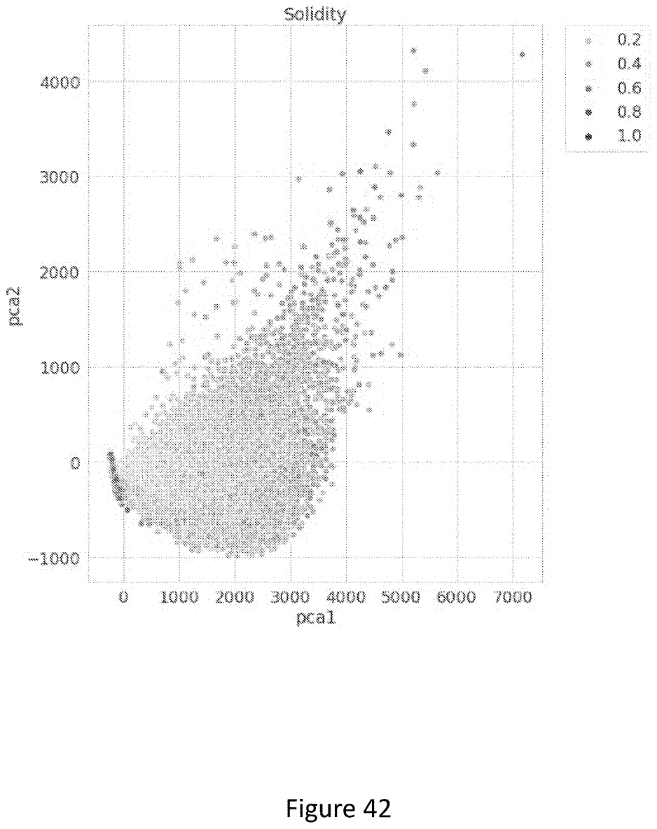

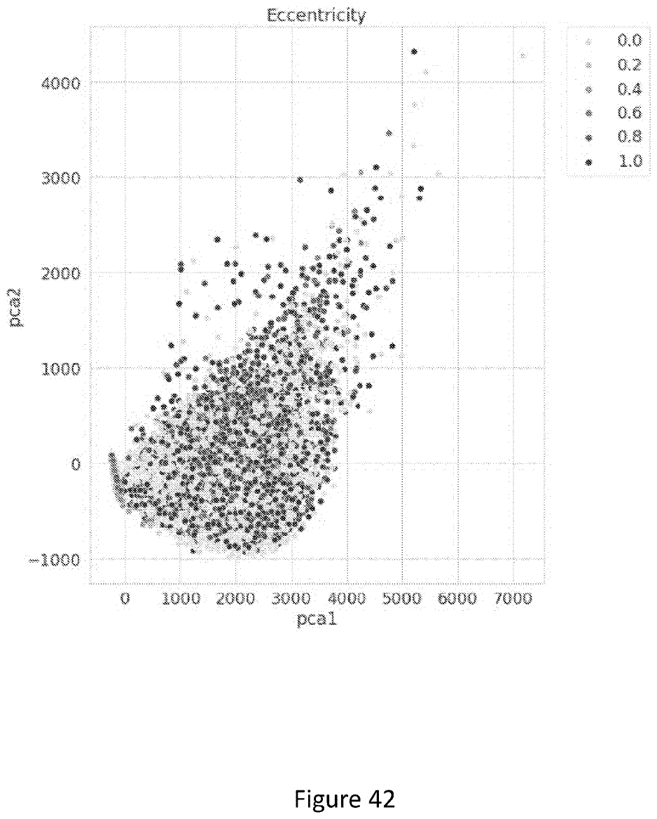

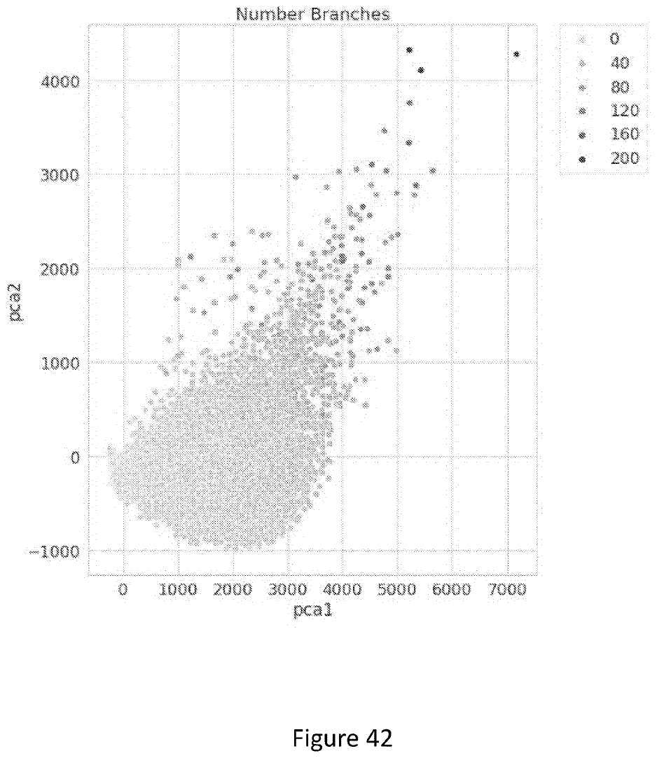

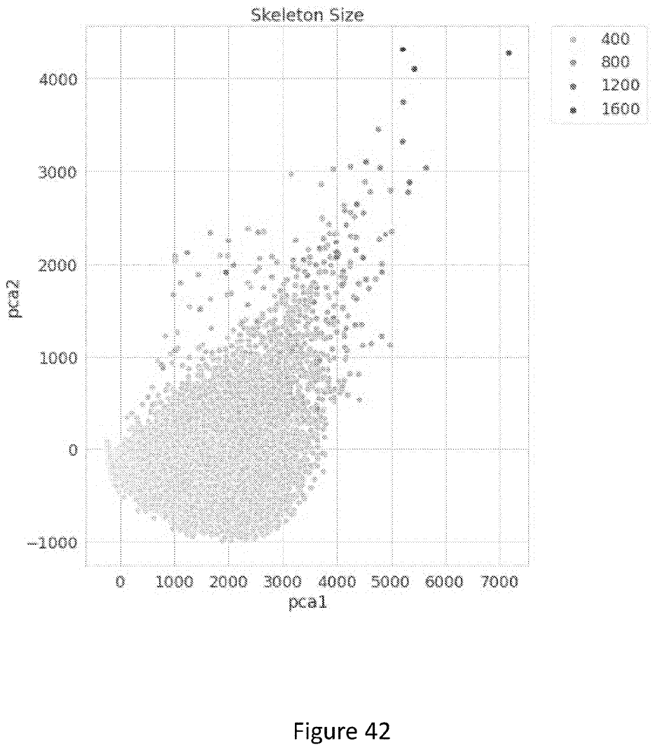

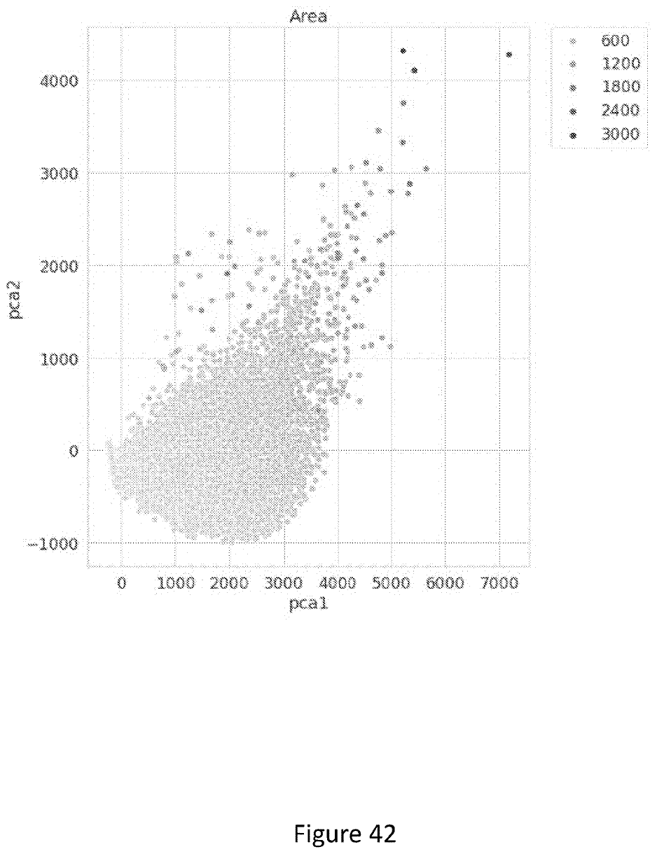

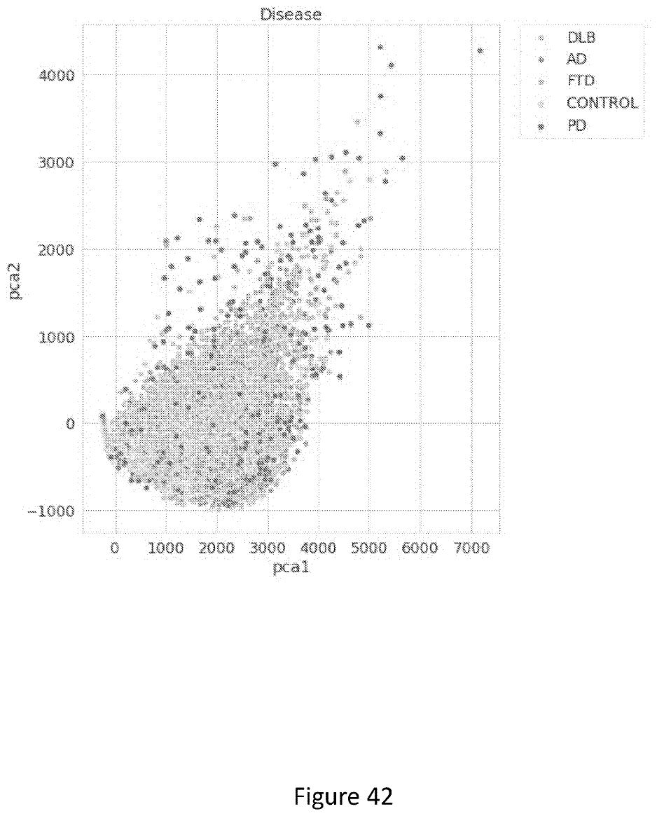

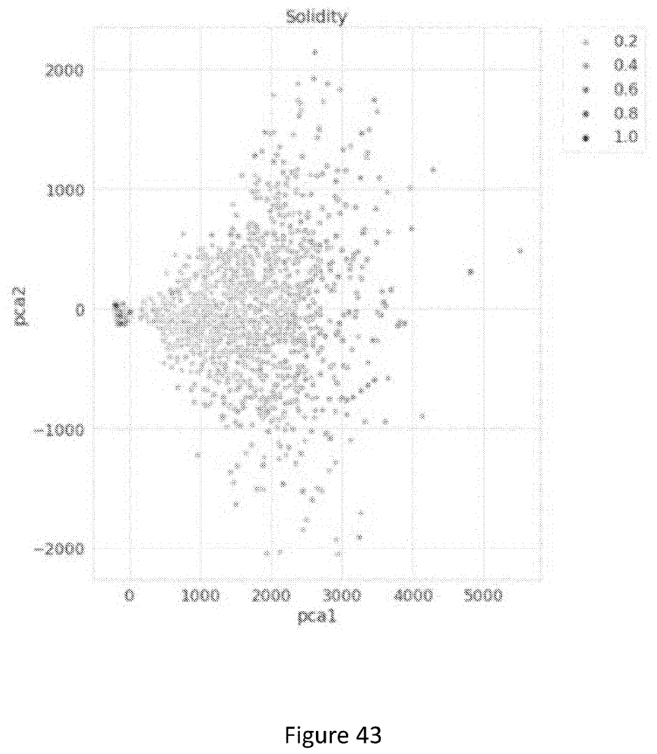

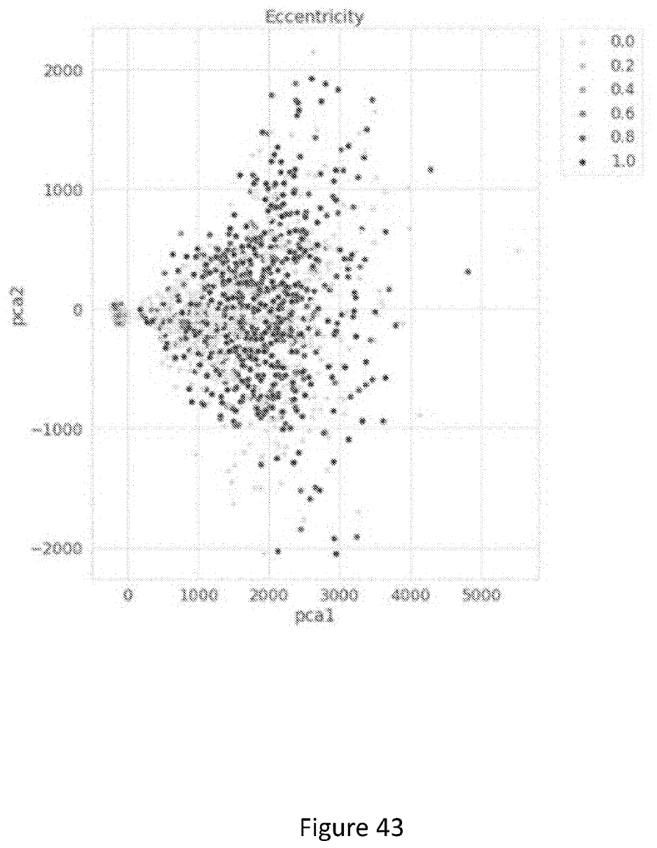

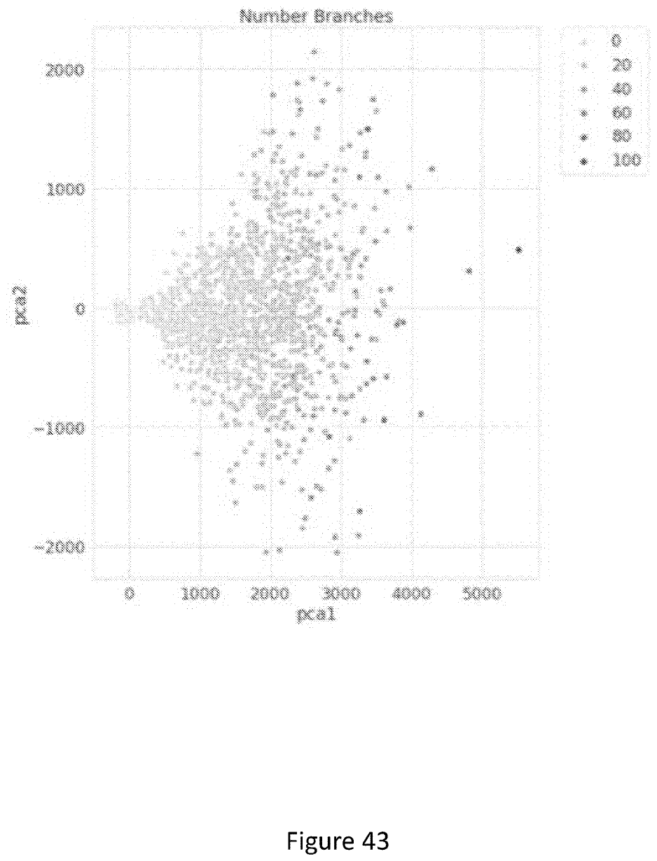

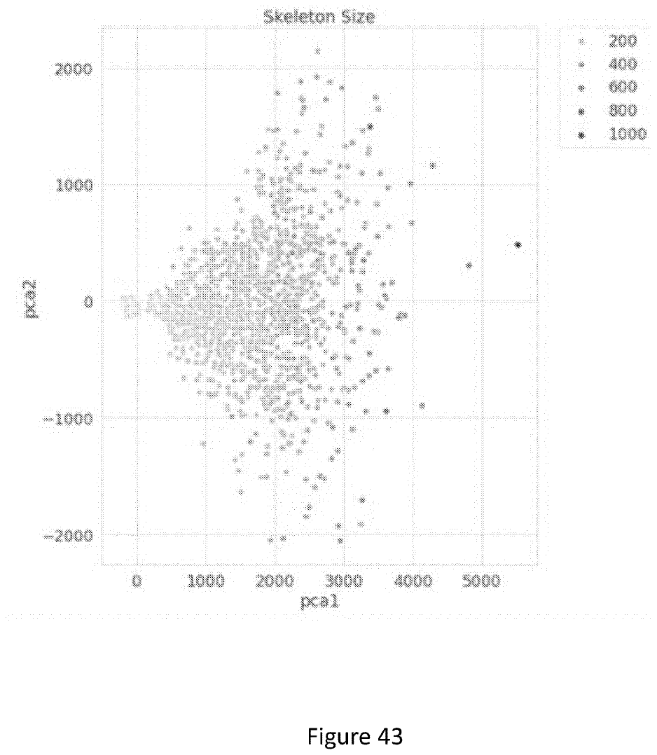

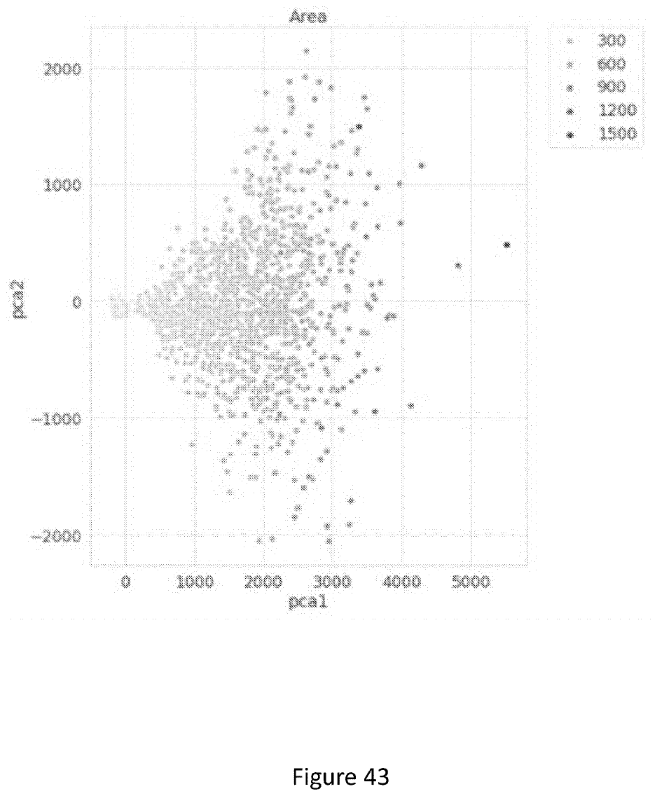

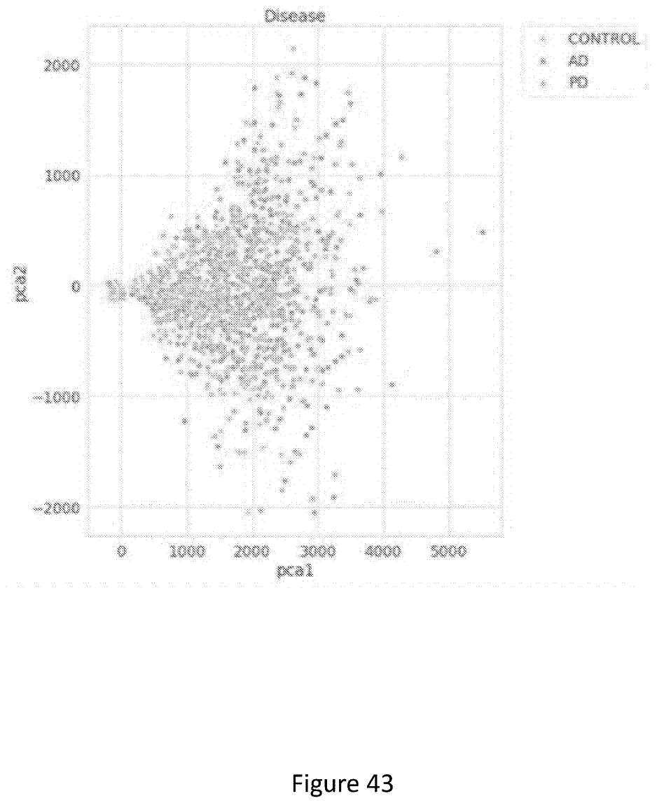

FIG. 42 shows plots illustrating the relationship between principal components and shape features/disease classes for the brain samples. Corresponding plots are shown in FIG. 43 for the CSF samples. The top-left scatter plot shows PC1 on the x-axis against PC2 on the y-axis, shaded by solidity. This plot visualises the distribution of aggregates based on their solidity values across the first two principal components. The top-centre scatter plot is shaded by eccentricity, illustrating the deviation of aggregate shapes from a perfect circle in relation to the major modes of variance in the dataset. In the top-right plot the data points are shaded by number of branches, illustrating how branching patterns of aggregates align with the variance captured by PC1 and PC2. The bottom-left scatter plot is shaded by skeleton size, indicating the relationship between the central structure or essential topology of the shapes and the significant variance captured by the first two principal components. The bottom-centre data points are coloured based on the area of the aggregates, representing how the size of the aggregates is distributed across the PCA space. In the bottom-right plot, the data is shaded by disease class.

### Density Maps and Disease Classification

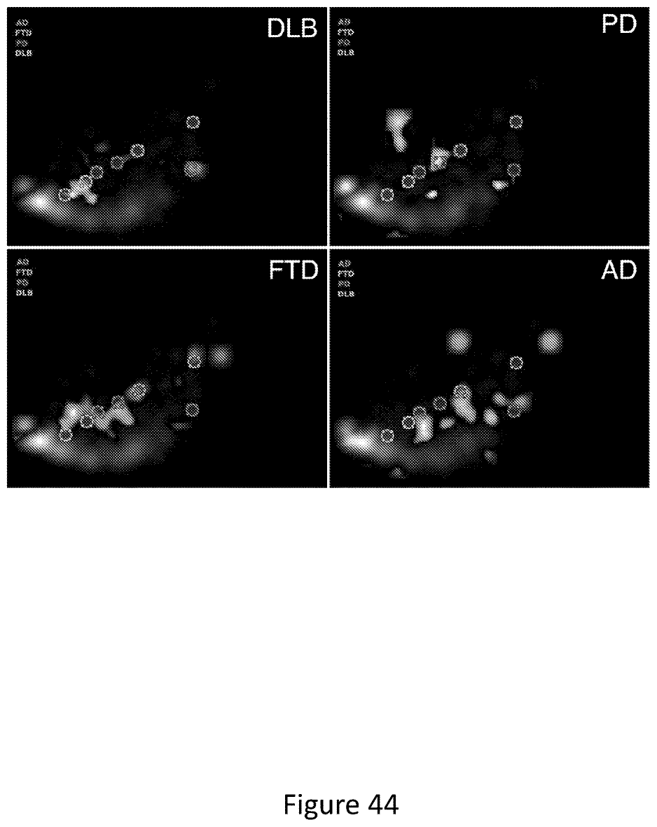

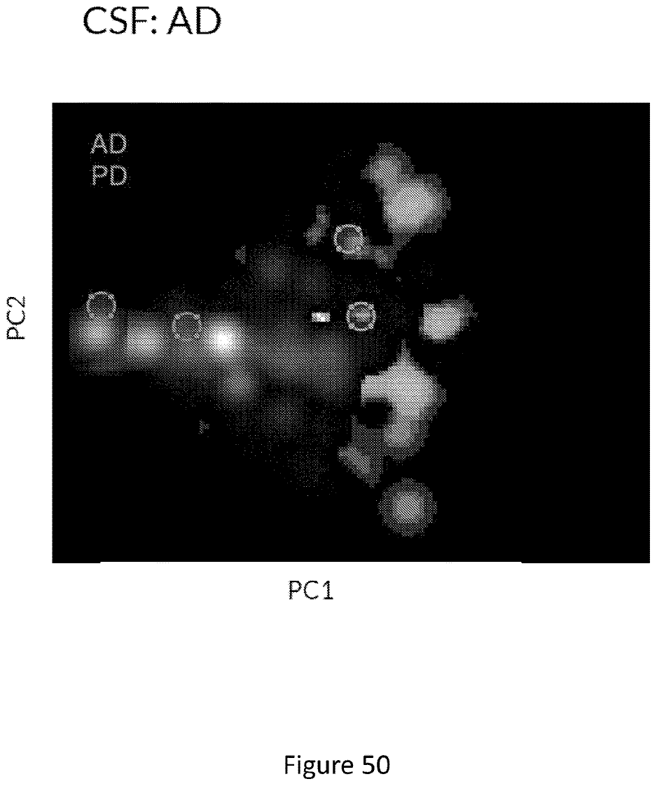

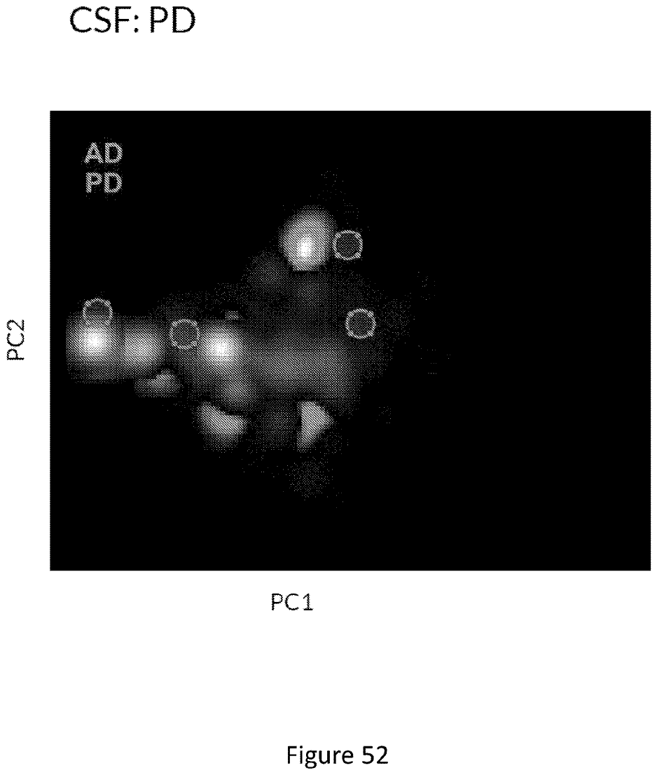

FIG. 44 show density maps constructed using the first and second principal components, for aggregates corresponding to DLB, PD, FTD and AD. As illustrated in FIG. 44, the position of each disease class on the PCA plot has been transformed into a density map, taking into account all the data points. The plots indicate the regions of highest concentration or intensity for each disease aggregate. A threshold corresponding to the mean value of the control samples plus two times the standard deviation is determined, and used to identify regions that are significant different from the controls. Disease data specific to the disease is then filtered out by comparison to the control—the disease data is compared to the threshold determined using the control data. Regions in the disease data that are significantly different from the control data can therefore be identified, and the centroid of these regions is marked (e.g. using the ‘regionprop’ function in matlab). The centre point of each circular representation pinpoints the average or ‘centre of gravity’ of data points related to specific diseases, derived from the mean values of their x and y coordinates (PC1 vs PC2). The circles have a radius of 3 units to represents regions with average diffusion, to prevent overcrowding and to ensure a direct comparison. Some regions match with the control mean (neutral regions, with no significant differences). Other regions show significant disease samples compared to controls, and in some regions the disease samples show less presences compared to the controls. Corresponding plots but for the CSF data are shown in FIG. 50 for AD and FIG. 52 for PD.

Remarkably, the positioning of each class remains consistent across all aggregates. Advantageously, using this knowledge of the specific locations where each disease is situated on the map, predictions regarding the disease classification for each aggregate can be made. The consistent spatial positioning of each disease class on the plot not only illuminates discernible patterns among the diseases but, more critically, furnishes a mechanism for informed disease classification for each aggregate. It provides a diagnostic tool, enabling a more predictive and systematic approach to disease classification at the aggregate level. Such precision can be instrumental in the early identification of diseases, potentially revolutionizing patient care and intervention strategies.

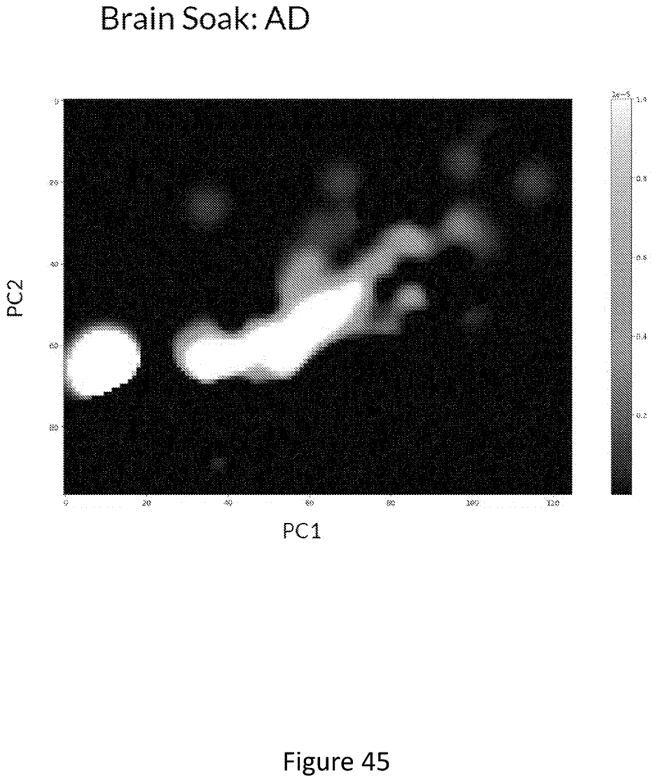

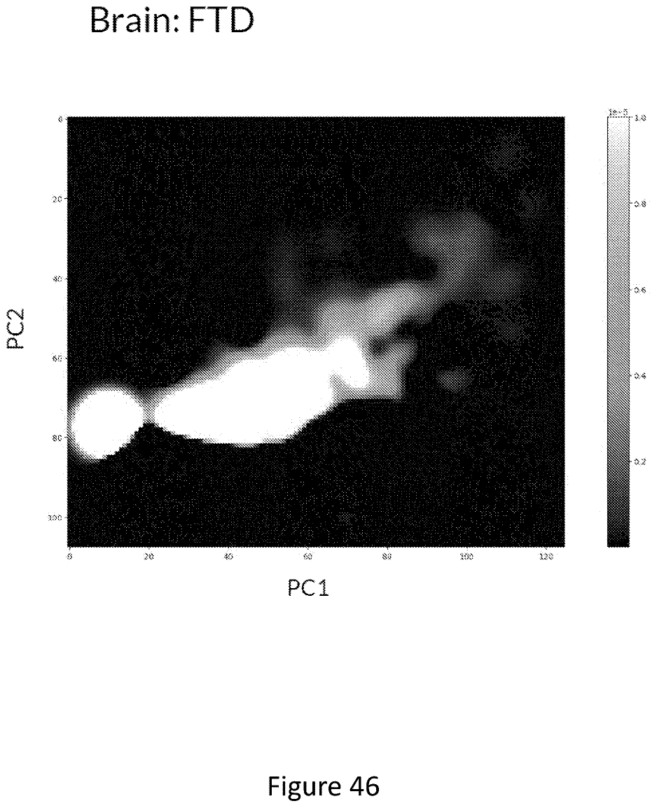

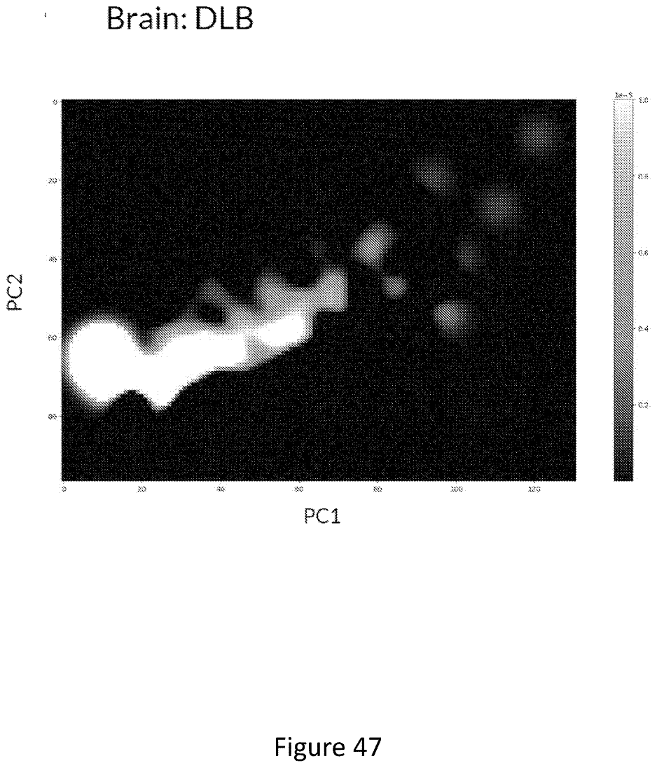

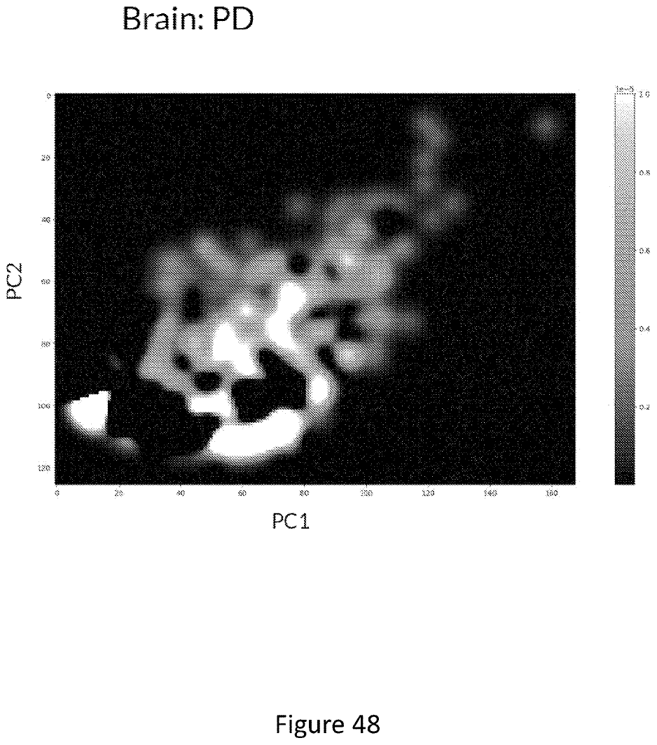

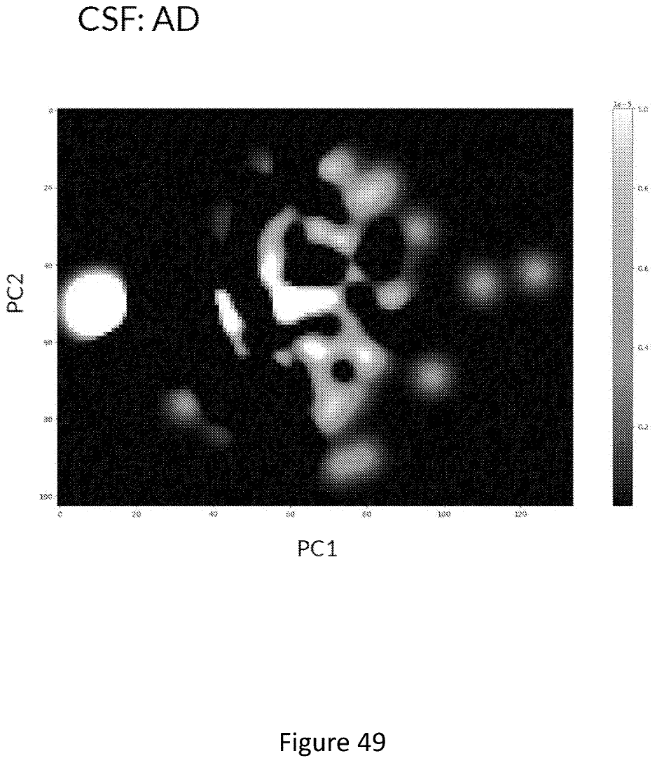

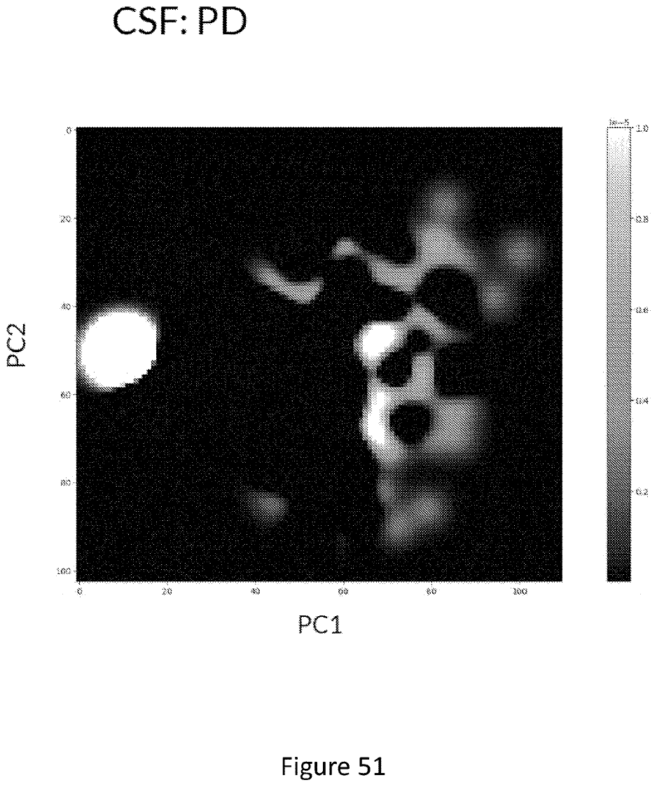

FIG. 45 shows a differential density map comparing AD and control groups in PCA space, for brain soak samples. FIGS. 46 to 48 show corresponding plots but for FTD, DLB and PD, respectively. The heatmaps graphically visualise the difference in data point concentrations between the disease and control when mapped in the PCA space across the first two principal components, PC1 and PC2. To generate these figures, gaussian smoothing was applied to the densities of data points for both groups. The smoothing technique helps in capturing the underlying distribution trends in the data, making them easier to visualise. Positive values (shown brighter) indicate regions where the disease group has a higher density compared to the control group. Conversely, darker shades represent region where the control group is more densely concentrated than the AD group. The scale quantifies the magnitude of this difference, with lighter shades denoting a higher difference in favour of the disease group and darker shades indicating a higher concentration for the control group. Corresponds plots for the CSF samples are shown in FIG. 49 for AD and in FIG. 51 for PD.

Using the specific positions for each disease class illustrated in FIG. 44, a combination of local maxima and nearest neighbour search (NNS) was employed for classification. Firstly, local maxima in the heat maps of FIG. 44 are determined. The local maxima are then utilised to extract corresponding values from the dataset (nearest neighbour search is restricted to only those points in the data set that are local maxima). Next, the nearest neighbour algorithm is used for classification, by identifying data that has the highest resemblance (or is “nearest”) to the observation, and then label of this most similar data is assigned. For each test sample, the Euclidean distance to every local maximum derived from the training data is calculated. This identifies which local maximum is closest to the test sample. Once identified, the label corresponding to this nearest local maximum is assigned to the test sample, ensuring that the test sample inherits the classification of its closest known neighbor in the feature space. However, it will be appreciated that NNS need not necessarily be used, and any other suitable classification algorithm could alternatively be used.

The precision, recall, and f1-score metrics for each class within the respective regions is shown in table 6. As shown in the table, better predictions of FTD are achieved using samples from the brain region, and better prediction of PD is achieved using samples from the CSF region.

TABLE 6
Classification report for brain and CSF samples.

| | Disease | Precision | Recall | F1-score |
|---|---|---|---|---|
| Brain | AD | 0.59 | 0.60 | 0.59 |
| | Control | 0.59 | 0.59 | 0.59 |
| | DLB | 0.64 | 0.62 | 0.63 |
| | FTD | 0.76 | 0.75 | 0.75 |
| | PD | 0.58 | 0.63 | 0.61 |
| CSF | AD | 0.79 | 0.80 | 0.80 |
| | Control | 0.73 | 0.75 | 0.74 |
| | PD | 0.85 | 0.84 | 0.85 |

When assessing the data from the brain region, the classification achieved an accuracy of 67.26%, and the accuracy for the CSF samples was 81.32%.

Advantageously, the inventors have found that highly accurate differential diagnosis can be achieved based on the morphology of protein aggregates when employing an autoencoder (e.g. convolutional autoencoder, in this example using 64 embedded values) to generate embedded values from images of the protein aggregates, and using PCA to analyse the embedded values and perform classification.

### Materials and Methods

### Data Source

Aggregates were isolated from post-mortem, frozen temporal cortical tissues, which were obtained from Parkinson's UK and Multiple Sclerosis Society Tissue Banks at Imperial College London and the Queen Square Brain Bank for Neurological Disorders at University College London.

### Aggregate Extraction

The Ye laboratory, (Imperial College London), implemented the summarized protocols to extract and image the aggregates. Brain samples underwent a soaking process in synthetic cerebrospinal fluid (CSF), followed by ultracentrifugation. The resulting supernatant, containing soluble aggregates, was subjected to 72 hours of dialysis and stored at −80° C. before imaging. >99.8% of aggregates used in the analyses had a size of up to 1 μm.

### SMLM Imaging of Aggregates

Aggregates were immobilised on a poly-L-lysine coated coverslip and imaged using on a total internal reflection fluorescence (TIRF) microscope. The imaging buffer contained PBS with 1 mg/ml glucose oxidase, 0.02 mg/ml catalase, 10% (w/w) glucose, and 100 mM methylamine (MEA). Aggregates were stained using Amytracker, a commercially available dye that significantly enhances its fluorescence emission when bound to aggregate structures. It was diluted at a ratio of 1:50,000 for single-molecule localisation microscopy (SMLM) experiments.

The TIRF microscope was an ECLIPSE Ti2-E inverted microscope (Nikon). A C-FLEX laser combiner (Hubner Photonics) and an optical fiber to couple the lasers to the microscope's E-TIRF arm were used to excite the Amytracker fluorophores. Additionally, a filter cube with a dichroic mirror and multiple filters was used to clean up the emission fluorescence and lower background noise. The microscope also employed a motorized filter turret and a Perfect focusing system by Nikon to reduce drift in the z-direction.

Images were recorded using an sCMOS camera at a rate of 20 Hz, and super-resolved images were reconstructed from movies typically two thousand frames in length. Average images of the aggregates on the coverslips were produced from a hundred frames. Morten, M. J. et al. PNAS; 119 (2022) provides a comprehensive description of the methodologies discussed.

### Pre-Processing: Cell Aggregate Image Reconstruction

SMLM images were reconstructed from the movies by a script performed using MATLAB version 9.14.0.2254940 (R2023a Update 2). A second MATLAB script was used to identify individual aggregates from a field of view, typically containing hundreds or thousands of aggregates. A Gaussian filter was used to define the background noise, and a rolling-ball filter was used to identify regions of interest (ROI) containing SMLM images of individual aggregates.

After collecting the image data from MATLAB, the images were then pre-processed for training a convolutional autoencoder. When defining a Convolutional autoencoder architecture, the number of layers depends on the size of the input. A fixed input shape was used for the model, and so the images were padded to 256×256 pixels. Images of less than 4 pixels were removed to avoid model training on indistinguishable images.

The data was then split to 80% training data and 20% test data. The split was performed at the patient level for each label of class (AD, PD, FTD, DLB and Control). This was done to ensure that all images from the same patient end up in the same subset (either training or test), which beneficially helps reduce the risk of overfitting and data leakage.

### Data

The data included images of cell aggregates from donors that were either healthy individuals, or patients who had been diagnosed with PD, DLB, FTD or AD. Samples were extracted from brain tissue and cerebrospinal fluid. In total, there were sixteen patients diagnosed with PD, five patients diagnosed with DLB, eight patients diagnosed with FTD, and thirteen patients diagnosed with AD. The cognitively healthy controls accounted for sixteen individuals. In table 7 and table 8, the specifics of the completed data set, including the total number of aggregates gathered from each individual patient, are shown. The data shown in tables 7 and 8 includes the data used to train the algorithms described above (e.g. the classifying neural network and the autoencoder).

To collect images of aggregates from each sample, the aggregates were incubated on a coverslip, and videos were recorded from approximately ten different fields of view of the coverslip. Each video was reconstructed so that a super-resolved image was created, and each of these images contains hundreds of aggregates with a pixel size of 12 nm. After applying thresholding to each SMLM image a total of 286,114 images were obtained from the brain region, and a total of 481,693 images from the cerebrospinal fluid of individual aggregates.

TABLE 7
Case details on the brain samples. The table represents a categorization
of subjects into distinct classes and their corresponding counts
from the training set of the brain soak samples. It consists of five
categories: Control, PD (Parkinson's Disease), DLB (Dementia with
Lewy Bodies), FTD (Frontotemporal Dementia), and AD (Alzheimer's
Disease). The “Counts by class” provides the total number
of aggregate images in each of these classes. Additionally, each
primary class is further subdivided by patients. Their respective
counts are presented under “Counts by patient.” The table
concludes with a cumulative count, which sums up all aggregate images,
amounting to 202,122 images in the brain soak training data samples.

| Class | Counts by class | Patient | Counts by patient |
|---|---|---|---|
| Control | 44,970 | Control 1 | 6119 |
| | | Control 2 | 3610 |
| | | Control 3 | 7108 |
| | | Control 4 | 8666 |
| | | Control 5 | 8251 |
| | | Control 6 | 8675 |
| | | Control 7 | 636 |
| | | Control 8 | 1905 |
| PD | 40,632 | PD 1 | 8675 |
| | | PD 2 | 8675 |
| | | PD 3 | 548 |
| | | PD 4 | 1026 |
| | | PD 5 | 5203 |
| | | PD 6 | 8675 |
| | | PD 7 | 904 |
| | | PD 8 | 6926 |
| DLB | 26,459 | DLB 1 | 3866 |
| | | DLB 2 | 8675 |
| | | DLB 3 | 8675 |
| | | DLB 4 | 5243 |
| FTD | 45,307 | FTD 1 | 8675 |
| | | FTD 2 | 8675 |
| | | FTD 3 | 8675 |
| | | FTD 4 | 1932 |
| | | FTD 5 | 8675 |
| | | FTD 6 | 8675 |
| AD | 44,754 | AD 1 | 8406 |
| | | AD 2 | 1646 |
| | | AD 3 | 8675 |
| | | AD 4 | 8675 |
| | | AD 5 | 8675 |
| | | AD 6 | 8675 |
| | | | Total: 202, 122 |

TABLE 8
Case details on the CSF samples
CSF Samples

| Patient | Age | Sex | Number of aggregates | Disease |
|---|---|---|---|---|
| C10 | 84 | F | 61694 | Control |
| C11 | 89 | M | 4650 | Control |
| C12 | 82 | M | 10141 | Control |
| C13 | 81 | F | 2853 | Control |
| C14 | 89 | M | 2853 | Control |
| PD11 | 78 | M | 22874 | PD |
| PD12 | 84 | M | 61694 | PD |
| PD13 | 83 | F | 61694 | PD |
| PD14 | 83 | F | 18778 | PD |
| PD15 | 80 | F | 64547 | PD |
| PD16 | 73 | M | 1900 | PD |
| AD9 | 88 | F | 41485 | AD |
| AD10 | 93 | M | 61694 | AD |
| AD11 | 87 | F | 61694 | AD |
| AD12 | 87 | F | 289 | AD |
| AD13 | 80 | M | 2853 | AD |

1. A method of screening for the presence of a proteopathy or an increased risk thereof in a patient, the method comprising:
(a) comparing a morphology, toxicity and/or abundance of protein aggregates in a patient sample to a control morphology, toxicity and/or abundance; and
(b) determining if the patient has a proteopathy or an increased risk thereof based on the comparison performed in step (a);
wherein the protein aggregates are up to 1 μm in size.

2-4. (canceled)

5. A method for determining the efficacy of a therapeutic intervention in a patient having a proteopathy, the method comprising:
(a) comparing: (i) the morphology, toxicity, and/or abundance of protein aggregates in a patient sample obtained prior to a therapeutic intervention with (ii) the morphology, toxicity, and/or abundance of protein aggregates in a patient sample obtained after the therapeutic intervention; and
(b) determining the efficacy of the therapeutic intervention based on the comparison performed in step (a);
wherein the protein aggregates are up to 1 μm in size.

6. The method of claim 1, wherein the proteopathy is a neurodegenerative disease.

7. (canceled)

8. The method of claim 1, wherein the protein aggregate is an amyloid-beta aggregate, an alpha-synuclein aggregate, a tau aggregate, a superoxide dismutase 1 (SOD1) aggregate, a TAR DNA-binding protein 43 (TDP-43) aggregate, a huntingtin (HTT) aggregate, and/or a prion/PrP aggregate.

9. (canceled)

10. The method of claim 1, wherein the proteopathy is selected from cancer, diabetes, cardiovascular disease, cystic fibrosis, sickle cell disease, protein aggregate myopathies and amyloidosis.

11. (canceled)

12. The method of claim 1, wherein the method comprises detecting and comparing protein aggregates up to 600 nm in size, optionally up to 550 nm, 540 nm, 530 nm, 520 nm, 510 nm, 500 nm, or 450 nm in size.

13-16. (canceled)

17. The method of claim 1, wherein the method comprises quantifying morphological features of the protein aggregates; and comparing said morphological features to a control.

18. (canceled)

19. The method of claim 1, wherein the method comprises quantifying the toxicity of the protein aggregates by: (a) performing a cell stress, viability and/or cytotoxicity assay; and/or (b) detecting proteasome foci formation.

20. The method of claim 1, wherein the method comprises comparing the relative abundance of the protein aggregates in the patient sample to a control, optionally wherein the abundance of the protein aggregates is determined relative to the total abundance of protein aggregates in the patient sample.

21-27. (canceled)

28. A computer program product for identifying and classifying a proteopathy in a patient, the computer program product comprising a computer readable storage medium having program instructions embodied therewith, the program instructions executable by a processor to cause the processor to perform a method comprising:
reading an input image of a protein aggregate;
determining, using a classification algorithm and the input, whether the protein aggregate is associated with a proteopathy or the severity, stage and/or prognosis associated with the protein aggregate; and
outputting an indication of whether the protein aggregate is associated with a proteopathy or the severity, stage and/or prognosis associated with the protein aggregate;
wherein the image of the protein aggregate is an image of a protein aggregate that is up to 1 μm in size.

29. The computer program product according to claim 28, wherein the classification algorithm comprises an artificial neural network;
wherein the input is received in an input layer of the neural network; and
wherein determining whether the protein aggregate is associated with a proteopathy comprises using the artificial neural network to determine whether the protein aggregate is associated with a proteopathy.

30-32. (canceled)

33. The computer program product according to claim 28, wherein the method comprises performing principal component analysis using the input to generate a set of variables, and determining whether the protein aggregate is associated with a proteopathy using the classification algorithm and the set of variables.

34. The computer program product according to claim 28, wherein the method comprises:
receiving the input in an input layer of an autoencoder, wherein the autoencoder is configured to generate an autoencoder output using the image of the protein aggregate; and
determining whether the protein aggregate is associated with a proteopathy by using the classification algorithm and the autoencoder output.

35-47. (canceled)

48. The method of claim 5, wherein the proteopathy is a neurodegenerative disease.

49. The method of claim 5, wherein the protein aggregate is an amyloid-beta aggregate, an alpha-synuclein aggregate, a tau aggregate, a superoxide dismutase 1 (SOD1) aggregate, a TAR DNA-binding protein 43 (TDP-43) aggregate, a huntingtin (HTT) aggregate, and/or a prion/PrP aggregate.

50. The method of claim 5, wherein the proteopathy is selected from cancer, diabetes, cardiovascular disease, cystic fibrosis, sickle cell disease, protein aggregate myopathies and amyloidosis.

51. The method of claim 5, wherein the method comprises detecting and comparing protein aggregates up to 600 nm in size, optionally up to 550 nm, 540 nm, 530 nm, 520 nm, 510 nm, 500 nm, or 450 nm in size.

52. The method of claim 5, wherein the method comprises quantifying morphological features of the protein aggregates; and comparing said morphological features to a control.

53. The method of claim 5, wherein the method comprises quantifying the toxicity of the protein aggregates by: (a) performing a cell stress, viability and/or cytotoxicity assay; and/or (b) detecting proteasome foci formation.

54. The method of claim 5, wherein the method comprises comparing the relative abundance of the protein aggregates in the patient sample to a control, optionally wherein the abundance of the protein aggregates is determined relative to the total abundance of protein aggregates in the patient sample.
